SEQUENCE LISTING (160 sequences)

SEQ ID NO: 1 - protein, 163 aa, Escherichia coli
MKLKAIILATGLINCIVFSAQAVDTTITVTGNVLQRTCNVPGNVDVSLGNLYVSDFPNAGSGSPWVNFDLSLTGCQNMNTVRATFSGTADGQTYYANTGNAGGIKIEIQDRDGSNASYHNGMFKTLNVQNNNATFNLKARAVSKGQVTPGNISSVITVTYTYA

SEQ ID NO: 2 - protein, 673 aa, Escherichia coli
MKMTRLYPLALGGLLLPAIANAQTSQQDESTLVVTASKQSSRSASANNVSSTVVSAPELSDAGVTASDKLPRVLPGLNIENSGNMLFSTISLRGVSSAQDFYNPAVTLYVDGVPQLSTNTIQALTDVQSVELLRGPQGTLYGKSAQGGIINIVTQQPDSTPRGYIEGGVSSRDSYRSKFNLSGPIQDGLLYGSVTLLRQVDDGDMINPATGSDDLGGTRASIGNVKLRLAPDDQPWEMGFAASRECTRATQDAYVGWNDIKGRKLSISDGSPDPYMRRCTDSQTLSGKYTTDDWVFNLISAWQQQHYSRTFPSGSLIVNMSQRWNQDVQELRAATLGDARTVDMVFGLYRQNTREKLNSAYDMPTMPYLSSTGYTTAETLAAYSDLTWHLTDRFDIGGGVRFSHDKSSTQYHGSMLGNPFGDQGKSNDDQVLGQLSAGYMLTDDWRVYTRVAQGYKPSGYNIVPTAGLDAKPFVAEKSINYELGTRYETADVTLQAATFYTHTKDMQLYSGPVGMQTLSNAGKADATGVELEAKWRFAPGWSWDINGNVIRSEFTNDSELYHGNRVPFVPRYGAGSSVNGVIDTRYGALMPRLAVNLVGPHYFDGDNQLRQGTYATLDSSLGWQATERMNISVYVDNLFDRRYRTYGYMNGSSAVAQVNMGRTVGINTRIDFF

SEQ ID NO: 3 - protein, 246 aa, Escherichia coli
MNKVFVVSVVAAACVFAVNAGAKEGKSGFYLTGKAGASVMSLSDQRFLSGDEEETSKYKGGDDHDTVFSGGIAVGYDFYPQFSIPVRTELEFYARGKADSKYNVDKDSWSGGYWRDDLKNEVSVNTLMLNAYYDFRNDSAFTPWVSAGIGYARIHQKTTGISTWDYEYGSSGRESLSRSGSADNFAWSLGAGVRYDVTPDIALDLSYRYLDAGDSSVSYKDEWGDKYKSEVDVKSHDIMLGMTYNF

SEQ ID NO: 4 - protein, 166 aa, Escherichia coli
MKLKAIILATGLINCIAFSAQAVDTTITVTGRVLPRTCTIGNGGNPNATVVLDNAYTSDLIAANSTSQWKNFSLTLTNCQNVNNVTSFGGTAENTNYYRNTGDATNIMVELQEQGNGNTPLKVGSTKVVTVSNGQATFNLKVRAVSKGNAGAGSINSQITVTYTYA

SEQ ID NO: 5 - protein, 1295 aa, Escherichia coli
MNKIYSLKYSAATGGLIAVSELAKRVSGKTNRKLVATMLSLAVAGTVNAANIDISNVWARDYLDLAQNKGIFQPGATDVTITLKNGDKFSFHNLSIPDFSGAAASGAATAIGGSYSVTVAHNKKNPQAAETQVYAQSSYRVVDRRNSNDFEIQRLNKFVVETVGATPAETNPTTYSDALERYGIVTSDGSKKIIGFRAGSGGTSFINGESKISTNSAYSHDLLSASLFEVTQWDSYGMMIYKNDKTFRNLEIFGDSGSGAYLYDNKLEKWVLVGTTHGIASVNGDQLTWITKYNDKLVSELKDTYSHKINLNGNNVTIKNTDITLHQNNADTTGTQEKITKDKDIVFTNGGDVLFKDNLDFGSGGIIFDEGHEYNINGQGFTFKGAGIDIGKESIVNWNALYSSDDVLHKIGPGTLNVQKKQGANIKIGEGNVILNEEGTFNNIYLASGNGKVILNKDNSLGNDQYAGIFFTKRGGTLDLNGHNQTFTRIAATDDGTTITNSDTTKEAVLAINNEDSYIYHGNINGNIKLTHNINSQDKKTNAKLILDGSVNTKNDVEVSNASLTMQGHATEHAIFRSSANHCSLVFLCGTDWVTVLKETESSYNKKFNSDYKSNNQQTSFDQPDWKTGVFKFDTLHLNNADFSISRNANVEGNISANKSAITIGDKNVYIDNLAGKNITNNGFDFKQTISTNLSIGETKFTGGITAHNSQIAIGDQAVVTLNGATFLDNTPISIDKGAKVIAQNSMFTTKGIDISGELTMMGIPEQNSKTVTPGLHYAADGFRLSGGNANFIARNMASVTGNIYADDAATITLGQPETETPTISSAYQAWAETLLYGFDTAYRGAITAPKATVSMNNAIWHLNSQSSINRLETKDSMVRFTGDNGKFTTLTVNNLTIDDSAFVLRANLAQADQLVVNKSLSGKNNLLLVDFIEKNGNSNGLNIDLVSAPKGTAVDVFKATTRSIGFSDVTPVIEQKNDTDKATWTLIGYKSVANADAAKKATLLMSGGYKAFLAEVNNLNKRMGDLRDINGESGAWARIISGTGSAGGGFSDNYTHVQVGADNKHELDGLDLFTGVTMTYTDSHAGSDAFSGETKSVGAGLYASAMFESGAYIDLIGKYVHHDNEYTATFAGLGTRDYSSHSWYAGAEVGYRYHVTDSAWIEPQAELVYGAVSGKQFSWKDQGMNLTMKDKDFNPLIGRTGVDVGKSFSGKDWKVTARAGLGYQFDLFANGETVLRDASGEKRIKGEKDGRMLMNVGLNAEIRDNLRFGLEFEKSAFGKYNVDNAINANFRYSF

SEQ ID NO: 6 - protein, 142 aa, Escherichia coli
MINIPSPTAVVMALVAISTLPSPSRVKLMPYPPRAHNTTGLLPVREICFPHHGDDGRNSIEPSISRAAHTDRLRFVCMTRTGSTTSRPFCPIPRSPALNASGQQDSGFWGVSSIPGDILMFQLHVLIVFICKINLSDNNISY

SEQ ID NO: 7 - protein, 318 aa, Escherichia coli
MYAREYRSTRPHKAIFFHLSCLTLICSAQVYAKPDMRPLGPNIADKGSVFYHFSATSFDSVDGTRHYRVWTAVPNTTAPASGYPILYMLDGNAVMDRLDDELLKQLSEKTPPVIVAVGYQTNLPFDLNSRAYDYTPAAESRKTDLHSGRFSRKSGGSNNFRQLLETRIAPKVEQGLNIDRQRRGLWGHSYGGLFVLDSWLSSSYFRSYYSASPSLGRGYDALLSRVTAVEPLQFCTKHLAIMEGSATQGDNRETHAVGVLSKIHTTLTILKDKGVNAVFWDFPNLGHGPMFNASFRQALLDISGENANYTAGCHELSH

SEQ ID NO: 8 - protein, 725 aa, Escherichia coli
MRINKILWSLTVLLVGLNSQVSVAKYSDDDNDETLVVEATAEQVLKQQPGVSVITSEDIKKTPPVNDLSDIIRKMPGVNLTGNSASGTRGNNRQIDIRGMGPENTLILIDGVPVTSRNSVRYSWRGERDTRGDTNWVPPEQVERIEVIRGPAAARYGSGAAGGVVNIITKRPTNDWHGSLSLYTNQPESSEEGATRRANFSLSGPLAGDALTTRLYGNLNKTDADSWDINSPVGTKNAAGHEGVRNKDINGVVSWKLNPQQILDFEVGYSRQGNIYAGDTQNSSSSAVTESLAKSGKETNRLYRQNYGITHNGIWDWGQSRFGVYYEKTNNTRMNEGLSGGGEGRILAGEKFTTNRLSSWRTSGELNIPLNVMVDQTLTVGAEWNRDKLDDPSSTSLTVNDRDISGISGSAADRSSKNHSQISALYIEDNIEPVPGTNIIPGLRFDYLSDSGGNFSPSLNLSQELGDYFKVKAGVARTFKAPNLYQSSEGYLLYSKGNGCPKDITSGGCYLIGNKDLDPEISVNKEIGLEFTWEDYHASVTYFRNDYQNKIVAGDNVIGQTASGAYILKWQNGGKALVDGIEASMSFPLVKERLNWNTNATWMITSEQKDTGNPLSVIPKYTINNSLNWTITQAFSASFNWTLYGRQKPRTHAETRSEDTGGLSGKELGAYSLVGTNFNYDINKNLRLNVGVSNILNKQIFRSSEGANTYNEPGRAYYAGVTASF

SEQ ID NO: 9 - protein, 1014 aa, Escherichia coli
MGNQWQQKYLLEYNELVSNFPSPERVVSDYIKNCFKTDLPWFSRIDPDNAYFICFSQNRSNSRSYTGWDHLGKYKTEVLTLTQAALINIGYRFDVFDDANSSTGIYKTKSADVFNEENEEKMLPSEYLHFLQKCDFAGVYGKTLSDYWSKYYDKFKLLLKNYYISSALYLYKNGELDEREYNFSMNALNRSDNISLLFFDIYGYYASDIFVAKNNDKVMLFIPGAKKPFLFKKNIADLRLTLKELIKDSDNKQLLSQHFSLYSRQDGVSYAGVNSVLHAIENDGNFNESYFLYSNKTLSNKDVFDAIAISVKKRSFSDGDIVIKSNSEAQRDYALTILQTILSMTPIFDIVVPEVSVPLGLGIITSSMGISFDQLINGDTYEERRSAIPGLATNAVLLGLSFAIPLLISKAGINQEVLSSVINNEGRTLNETNIDIFLKEYGIAEDSISSTNLLDVKLKSSGQHVNIVKLSDEDNQIVAVKGSSLSGIYYEVDIETGYEILSRRIYRTEYNNEILWTRGGGLKGGQPFDFESLNIPVFFKDEPYSAVTGSPLSFINDDSSLLYPDTNPKLPQPTSEMDIVNYVKGSGSFGDRFVTLMRGATEEEAWNIASYHTAGGSTEELHEILLGQGPQSSLGFTEYTSNVNSADAASRRHFLVVIKVHVKYITNNNVSYVNHWAIPDEAPVEVLAVVDRRFNFPEPSTPPDISTIRKLLSLRYFKESIESTSKSNFQKLSRGNIDVLKGRGSISSTRQRAIYPYFEAANADEQQPLFFYIKKDRFDNHGYDQYFYDNTVGLNGIPTLNTYTGEIPSDSSSLGSTYWKKYNLTNETSIIRVSNSARGANGIKIALEEVQEGKPVIITSGNLSGCTTIVARKEGYIYKVHTGTTKSLAGFTSTTGVKKAVEVLELLTKEPIPRVEGIMSNDFLVDYLSENFEDSLITYSSSEKKPDSQITIIRDNVSVFPYFLDNIPEHGFGTSATVLVRVDGNVVVRSLSESYSLNADASEISVLKVFSKKF

SEQ ID NO: 10 - protein, 454 aa, Escherichia coli
MVDMINESARQTPVIAQTDVLVIGGGPAGLSAAIAAGRLGARTMIVERYGSLGGVLTQVGVESFAWYRHPGTEDCEGICREYEGRARALGFTRPEPQSISEVIDTEGFKVVADQMITESGVEPLYHSWVVDVIKDGDTLCGVIVENKSGRGAILAKRIVDCTGDADIAARAGAPWTKRSKDQLMGVTVMFSCAGVDVARFNRFVAEELKPTYADWGKNWTIQTTGKEDPMFSPYMEDIFTRAQQDGVIPGDAQAIAGTWSTFSESGEAFQMNMVYAFGFDCTDVFDLTKAEIAGRQQALWAIDALRHYVPGFENVRLRNFGATLGTRESRLIEGEIRIADDYVLNQGRCSDSVGIFPEFIDGSGYLILPTTGRFFQIPYGCLVPQKVENLLVAGRCISAGVVAHTSMRNMMCCAVTGEAAGTAAVVSLQQNCTVRQVAIPDLQNTLQQQGVRLA

SEQ ID NO: 11 - protein, 253 aa, Escherichia coli
MSAKRRLLIACTLITAIYHFPAYSSLEYKGTFGSINAGYADWNSGFVNTHRGEVWKVTADFGVNFKEAEFYSFYESNVLNHAVAGRNHTVSAMTHVRLFDSDMTFFGKIYGQWDNSWGDDLDMFYGFGYLGWNGEWGFFKPYIGLHNQSGDYVSAKYGQTNGWNGYVVGWTAVLPFTLFDEKFVLSNWNEIELDRNDAYTEQQFGRNGLNGGLTIAWKFYPRWKASVTWRYFDNKLGYDGFGDQMIYMLGYDF

SEQ ID NO: 12 - protein, 492 aa, Escherichia coli
MASLIGLAVCTGNAFSPALAAEAKQPNLVIIMADDLGYGDLATYGHQIVKTPNIDRLAQEGVKFTDYYAPAPLSSPSRAGLLTGRMPFRTGIRSWIPSGKDVALGRNELTIANLLKAQGYDTAMMGKLHLNAGGDRTDQPQAQDMGFDYSLANTAGFVTDATLDNAKERPRYGMVYPTGWLRNGQPTPRADKMSGEYVSSEVVNWLDNKKDSKPFFLYVAFTEVHSPLASPKKYLDMYSQYMSAYQKQHPDLFYGDWADKPWRGVGEYYANISYLDAQVGKVLDKIKAMGEEDNTIVIFTSDNGPVTREARKVYELNLAGETDGLRGRKDNLWEGGIRVPAIIKYGKHLPQGMVSDTPVYGLDWMPTLAKMMNFKLPTDRTFDGESLVPVLEQKALKREKPLIFGIDMPFQDDPTDEWAIRDGDWKMIIDRNNKPKYLYNLKSDRYETLNLIGKKPDIEKQMYGKFLKYKTDIDNDSLMKARGDKPEAVTWG

SEQ ID NO: 13 - protein, 345 aa, Escherichia coli
LISLSFIPVMSALPGPIAKGFRNERGFVTTTICAMGELLAEFLSRNPHQKFTQPGEFIGPFPSGAPAIFAAQVAKLSHRAIFFGCVGNDDFARLIIERLRHEGVITDGIHVMNNAVTGTAFVSYQNPQQRDFVFNIPNSACGLFTAEHIDKDLLKQCNHLHIVGSSLFSFRMIDVMRKAITTIKSAGGTVSFDPNIRKEMLSIPEMAQALDYLIEYTDIFIPSESELPFFARHKNLSEEQIVSDLLHGGVKHVAIKRAQRGASYYKLKNGTLHAQHVAGHDIEIIDPTGAGDCFGATFITLFLSGFPAHKALQYANASGALAVMRQGPMEGISSLADIEDFLQQH

SEQ ID NO: 14 - protein, 192 aa, Escherichia coli
MYMPGKQMLCCILISIISEGDMKIFISLFLFIISTNSFADDITHAGVVRIEGLITEKTCIISDESKNFTVNMPDVPSSSVRSAGDVTEKVYFSITLTRCGSDVGNAYIKFTGNTVSEDASLYKLEDGSVEGLALTIFDKNKGSISNDVKSMVFSLTSSVDNILHFFAAYKALKNNVQPGDANASVSFIVTYD

SEQ ID NO: 15 - protein, 201 aa, Escherichia coli
MIKFRLYIPPVILGFVIVPLLVWPTVIALAVLIFTLTFLAEIIFSFPLLVVRISLQELQLELLVVYALFFSVMGGIGWQFSRRTPPELKNRLHCWLVFSPVYFWLILSNFILYISPEKSALLENIRNFFLTFVWLPLNFSPFWPQPWTDFVGPISAQLGFALGYYCQWRSKNRSHRKKWGDWVTCLSLAILALGPLFNYLQ

SEQ ID NO: 16 - protein, 234 aa, Escherichia coli
MKFNLSNLSAVLLASGMLMSTAVTAAPGDATQFGGADTDWSTVDYPRLTDMDDNVDSMGGKIRFTGRVVKATCKVATDSKQIEVVLPVVPSNLFTGIDVEAQGASNQTDFNINLTECSNTDDQKIEFRFTGTADSANKTLANEVEGSTDADNSGNAGATGVGIRIYSKGTTNNGLINLNTTAAEGSASTAAYTIPGNATTHDFSAAFTAGYAQNGSTVAPGVVKSTASFVVLYE

SEQ ID NO: 17 - protein, 336 aa, Escherichia coli
MRIHTYWYRRYFILLIIIFSNVLSSIANAEDMGRERAYCYPGSPSNNTTPASFSYNFGTIVVSDVNKNAPGTVLPSQIWKVGTYKAYCNSLDDYEIYFSAVSGIDPSGASGDHQGSDVFIPLTHEISVSTHIKLYNQNGTMTDKIVPFENYNTNYPGDRSKPSNWASGTEGYIKIRIDKKIISDVSLSNVLLVSLYVSQIPTEHGPIPVFNAYIGNLNIQVPQGCTINEGTSFTVNMPDVWASELSRAGAGAKPAGVTPVATTIPINCTNKDTDAVMTLVFDGNISATRDTNGKQSIIQAQDNPDVGIMIMDSQQNSVDLNALATSVGVPFRLVEN

SEQ ID NO: 18 - protein, 864 aa, Escherichia coli
MNLKLKRCEYWMAAQKQMKRVVPLLLVIMPACSIAGMRFNPAFLSGDTEAVADLSRFEKGMTYLPGSYEVEVWVNDSPLLSRTVTFKADDENQLIPCLSLADLLSLGINKNALPEQALASSENSCLDLRIWFPDVHYMPELDAQRLKLTFPQAIIKRDARGYIPPEQWDNGITAFLLNYDFSGNNDRGDYSSNNYYLNLRAGINIGAWRFRDYSTWSRGSNSAGKLEHISSTLQRVIIPFRSELTLGDTWSSSDVFDSVSIRGIKLESDENMLPDSQSGFAPTVRGIAKSRAQVTIKQNGYVIYQTYMPPGPFEISDLNPTSSAGDLEVTIKESDNSETVYTVPYAAVPILQREGHLKYSTTVGQYRSNSYNQKSPYVFQGELIWGLPWDITAYGGAQFSEDYRALALGLGLNLGVFGATSFDVTQANSSLVDGSKHQGQSYRFLYSKSLVQTGTAFHIIGYRYSTQGFYTLSDTTYQQMSGTVVDPKTLDDKDYVYNWNDFYNLRYSKRGKFQASVSQPFGNYGSMYLSASQQTYWNTDKKDSLYQVGYNTSIKGIYLNVAWNYSKSPGTNADKIVSLNVSLPISNWLSSTNDGRSSSNAMTATYGYSQDNHGQVNQYTGVSGSLLEQHNLSYNIQHGFANQDNSSSGSVGVNYRGAYGSLNSAYSYDNEGNQQINYGISGALVVHENGLTLSQPLGETNVLIKAPGANNVDVQRGTGISTDWRGYAVVPYATEYRRNNISLDPMSMNMHTELDITSTEVIPGKGALVRAEFAAHIGIRGLFTVRYRNKSVPFGATASAQIKNSSQITGIVGDNGQLYLSGLPLEGVINIQWGDGVQQKCQANYKLPETELDNPVSYATLECR

SEQ ID NO: 19 - protein, 169 aa, Escherichia coli
MGAIYVKRLILSVALIIPIASNASDALNQPSSSLNDGVETFFISCFDMPQETTTDMDACQRVQLAQVSWVKNKYSVAALNRLKQDNKDDPQRLQELTASFNAESEAWTELIEKASKSVQVDYVGGTIAGTAVASRQIGLLELQSHDIWEHWLRSRGLNSSSFARTKVQI

SEQ ID NO: 20 - protein, 713 aa, Escherichia coli
MAMFTPSFSGLKGRALFSLLFAAPMIHATDSVTTKDGETITVTADANTATEATDGYQPLSTSTATLTDMPMLDIPQVVNTVSDQVLENQNATTLDEALYNVSNVVQTNTLGGTQDAFVRRGFGANRDGSIMTNGLRTVLPRSFNAATERVEVLKGPASTLYGILDPGGLINVVTKRPEKTFHGSVSATSSSFGGGTGQLDITGPIEGTQLAYRLTGEVQDEDYWRNFGKERSTFIAPSLTWFGDNATVTMLYSHRDYKTPFDRGTIFDLTTKQPVNVDRKIRFDEPFNITDGQSDLAQLNAEYHLNSQWTARFDYSYSQDKYSDNQARVTAYDATTGTLTRRVDATQGSTQRMHATRADLQGNVDIAGFYNEILGGVSYEYYDLLRTDMIRCKKAKDFNIYNPVYGNTSKCTTVSASDSDQTIKQENYSAYAQDALYLTDNWIAVAGIRYQYYTQYAGKGRPFNVNTDSRDEQWTPKLGLVYKLTPSVSLFANYSQTFMPQSSIASYIGDLPPESSNAYEVGAKFELFDGITADIALFDIHKRNVLYTESIGDETIAKTAGRVRSRGVEVDLAGALTENINIIASYGYTDAKVLEDPDYAGKPLPNVPRHTGSLFLTYDIHNMPGNNTLTFGGGGHGVSRRSATNGADYYLPGYFVADAFAAYKMKLQYPVTLQLNVKNLFDKTYYTSSIATNNLGNQIGDPREVQFTVKMEF

SEQ ID NO: 21 - protein, 606 aa, Escherichia coli
MKISWNYIFKNKWRFHITSISLFLIMLAVSIAFLHLRFNTLSSTDKMRLEMYKSTLYSTIEQFYVLPYMLSTDHIIRQAVITPDDMTSSELNQRIAHFNTQLKTAAIFILDTQGKAIASSNWQDPGSYVGQNYSYRPYYKHAMSGLNGRFYGIGSTTNTPGFFLSTSIKDKGKIVGVVVVKISLNEIEKAWAEGPENIIVNDEHGIIFLSSKSPWRMRTLQPLPVQAKQKLQSTRQYSLDNLLPADYYPCYTVSNFTFLKDKKEQLCLFPQYYTQQIAIPEFNWKMTIMVPLDNLYWSWAISLVITLIIYLLFLLFIKYWRMRSHAQQLLTLANETLEKQVKERTSALELINQKLIQEIKERSQAEQVLQITRSELAESSKLAALGQMATEIAHEQNQPLAAIHALTDNARTMLKKEMYPQVEQNLKHIISVIERMTQLISELKAFASRHRVPKGSADVIKVMYSAVALLNHSMEKNNIERRIKAPSMPLFVNCDELGLEQIFSNLISNALDSMEGSSYKRLDIAIRQANNKVIITIKDSGGGFAPEVVDRIFEPFFTTKRRGMGLGLAIVSEIVRNSNGALHASNHPEGGAVMTLTWPEWGEEHE

SEQ ID NO: 22 - protein, 101 aa, Escherichia coli
VLTPQHLRCVLTCSDLLTLLSGTVMSQMPLYFLNTQKKLTAHYEWLQINLTDTYELVKRLMPIPSLDVVVKVGKLVLPEKGHHGFYPEAGVVYRTVAPENP

SEQ ID NO: 23 - protein, 263 aa, Escherichia coli
MMKNTGYILALCLTASGHVLAHDVWITGKQAENNVTAEIGYGHNFPSKGTIPDRRDFFENPRLYNGKETITLKPASTDYVYKTESASKDNGYVLSTYMKPGYWSRTSSGWKPVSREGRNDVAYCEFVTKYAKSFIPGEQQMPAQLYQSPTGHELEIIPLSDISRFSENVKLKVLYKTSPLAGAIMELDSVSYLTSSRHTHAVEHKHPVHKAELTFVTNEDGIVTVPSLHIGQWLAKVQNKKSFQDKSLCDETVDVATLSFSRN

SEQ ID NO: 24 - protein, 378 aa, Escherichia coli
MGKIKYWLIVGFIILFAIFYIAISDRDSTLSRLKSAGENGDVEAQYALGLMYLYGEILDVDYQQAKIWYEKAADQNDPRAQAKLGVMYANGLGVNQDYQQSKLWYEKAAAQNDVDAQFLLGEMYDDGLGVSQDYQHAKMWYEKAAAQNDERAQVNLAVLYAKGNGVEQDYRQAKSWYEKAAAQNSPDAQFALGILYANANGVEQDYQQAKDWYEKAAEQNFANAQFNLGMLYYKGEGVKQNFRQAREWFEKAASQNQPNAQYNLGQIYYYGQGVTQSYRQAKDWFEKAAEKGHVDAQYNLGVIYENGEGVSQNYQQAKAWYEKAASQNDAQAQFELGVMNELGQGESIDLKQARHYYERSCNNGLKKGCERLKELLYK

SEQ ID NO: 25 - protein, 654 aa, Escherichia coli
MNVIRTVICTLIILPVGLQAATSHSSMVKDTITIVATGNQNTVFETPSMVSVVTNDTPWSQNAVTSAGMLKGVAGLSQTGAGRTNGQTFNLRGYDKSGVLVLVDGVRQLSDMAKSSGTYLDPALVKRIEVVRGPNSSLYGSGGLGGVVDFRTADAADFLPPGETNGLSLWGNIASGDHSTGSGLTWFGKTGKTDALLSVIMRKRGNIYQSDGEHAPNKEKPAALFAKGSVGITDSNKAGASLRLYRNNTTEPGNSTQTHGDSGLRDRKTVQNDVQFWYQYAPVDNSLINVKSTLYLSDITIKTNGHNKTAEWRNNRTSGVNVVNRSHTLIFPGAHQLSYGAEYYRQQQKPEGSATLYPEGNIDFTSLYFQDEMTMKSYPVNIIVGSRYDRYKSFNPRAGELKAERLSPRAAISVSPTDWLMMYGSISSAFRAPTMAEMYRDDVHFYRKGKPNYWVPNLNLKPENNITREIGAGIQLDGLLTDNDRLQLKGGYFGTDARNYIATRVDMKRMRSYSYNVSRARIWGWDMQGNYQSDYVDWMLSYNRTESMDASSREWLGSGNPDTLISDISIPVGHRGVYAGWRAELSASATHVKKGDPHQAGYTIHSFSLSYKPVSVKGFEASVTLDNAFNKLAMNGKGVPLSGRTVSLYTRYQW

SEQ ID NO: 26 - protein, 1376 aa, Escherichia coli
MNKIYALKYCYITNTVKVVSELARRVCKGSTRRGKRLSVLTSLALSALLPTVAGASTVGGNNPYQTYRDFAENKGQFQAGATNIPIFNNKGELVGHLDKAPMVDFSSVNVSSNPGVATLINPQYIASVKHNKGYQSVSFGDGQNSYHIVDRNEHSSSDLHTPRLDKLVTEVAPATVTSSSTADILNPSKYSAFYRAGSGSQYIQDSQGKRHWVTGGYGYLTGGILPTSFFYHGSDGIQLYMGGNIHDHSILPSFGEAGDSGSPLFGWNTAKGQWELVGVYSGVGGGTNLIYSLIPQSFLSQIYSEDNDAPVFFNASSGAPLQWKFDSSTGTGSLKQGSDEYAMHGQKGSDLNAGKNLTFLGHNGQIDLENSVTQGAGSLTFTDDYTVTTSNGSTWTGAGIIVDKDASVNWQVNGVKGDNLHKIGEGTLVVQGTGVNEGGLKVGDGTVVLNQQADSSGHVQAFSSVNIASGRPTVVLADNQQVNPDNISWGYRGGVLDVNGNDLTFHKLNAADYGATLGNSSDKTANITLDYQTRPADVKVNEWSSSNRGTVGSLYIYNNPYTHTVDYFILKTSSYGWFPTGQVSNEHWEYVGHDQNSAQALLANRINNKGYLYHGKLLGNINFSNKATPGTTGALVMDGSANMSGTFTQENGRLTIQGHPVIHASTSQSIANTVSSLGDNSVLTQPTSFTQDDWENRTFSFGSLVLKDTDFGLGRNATLNTTIQADNSSVTLGDSRVFIDKKDGQGTAFTLEEGTSVATKDADKSVFNGTVNLDNQSVLNINEIFNGGIQANNSTVNISSDSAVLENSTLTSTALNLNKGANVLASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNTWSGSLNAPDATVSMTDTQWSMNGNSTAGNMKLNRTIVGFNGGTSSFTTLTTDNLDAVQSAFVMRTDLNKADKLVINKSATGHDNSIWVNFLKKPSDKDTLDIPLVSAPEATADNLFRASTRVVGFSDVTPTLSVRKEDGKKEWVLDGYQVARNDGQGKAAATFMHISYNNFITEVNNLNKRMGDLRDINGEAGTWVRLLNGSGSADGGFTDHYTLLQMGADRKHELGSMDLFTGVMATYTDTDASAGLYSGKTKSWGGGFYASGLFRSGAYFDLIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDGRMLYGVGLNARFGDNTRLGLEVERSAFGKYNTDDAINANIRYSF

SEQ ID NO: 27 - protein, 349 aa, Escherichia coli
MITLFRLLAILCLFFNVSAFAVDCYQDGYRGTTLINGDLPTFKIPENAQPGQKIWESGDINITVYCDNAPGWSSNNPSENVYAWIKLPQINSADMLNNPYLTFGVTYNGVDYEGTNEKIDTHACLDKYEQYYNGYYHDPVCNGSTLQKNVTFNAHFRVYVKFKSRPAGDQTVNFGTVNVLQFDGEGGANMAPNAKNLRYAITGLDNISFLDCSVDVRISPESQIVNFGQIAANSIATFPPKAAFSVSTIKDIASDCTEQFDVATSFFTSDTLYDNTHLEIGNGLLMRITDQKTQEDIKFNQFKLFSTYIPGQSAAMATRDYQAELTQKPGEPLVYGPFQKDLIVKINYH

SEQ ID NO: 28 - protein, 840 aa, Escherichia coli
MNNKNTFSRDKLSHAIKNALSGVVCSLLFVLPVHAVEFNVDMIDAEDRENIDISRFEKKGYIPPGRYLVRVQINKNMLPQTLILEWVKADNESGSLLCLTKENLTNFGLNTEFIESLQNIAGSECLDLSQRQELTTRLDKATMILSLSVPQAWLKYQATNWTPPEFWDTGITGFILDYNVYASQYAPHHGDSTQNVSSYGTLGFNLGAWRLRSDYQYNQNFADGRSVNRDSEFARTYLFRPIPSWSSKFTMGQYDLSSNLYDTFHFTGASLESDESMLPPDLQGYAPQITGIAQTNAKVTVAQNGRVLYQTTVAPGPFTISDLGQSFQGQLDVTVEEEDGRTSTFQVGSASIPYLTRKGQVRYKTSLGKPTSVGHNDINNPFFWTAEASWGWLNNVSLYGGGMFTADDYQAITTGIGFNLNQFGSLSFDVTGADASLQQQNSGNLRGYSYRFNYAKHFESTGSQITFAGYRFSDKDYVSMSEYLSSRNGDESIDNEKESYVISLNQYFETLELNSYLNVTRNTYWDSASNTNYSVSVSKNFDIGDFKGISASLAVSRIRWDDDEENQYYFSFSLPLQQNRNISYSMQRTGSSNTSQMISWYDSSDRNNIWNISASATDDNIRDGEPTLRGSYQHYSPWGRLNINGSVQPNQYNSVTAGWYGSLTATRHGVALHDYSYGDNARMMVDTDGISGIEINSNRTVTNGLGIAVIPSLSNYTTSMLRVNNNDLPEGVDVENSVIRTTLTQGAIGYAKLNATTGYQIVGVIRQENGRFPPLGVNVTDKATGKDVGLVAEDGFVYLSGIQENSILHLTWGDNTCEVTPPNQSNISESAIILPCKTVK

SEQ ID NO: 29 - protein, 169 aa, Escherichia coli
LMNTKQSVAQLAVPHRKRLSSTMVVALLLCVVAGAVMINAADFPATAIETDPGASAFPTFYACALIVLAVLLVIRDLLQAKPASCANAQEKPAFRKTATGIAATAFYIVAMSYCGYLITTPVFLIVIMTLMGYRRWVLTPGIALLLTAILWLLFVEALQVPLPVGTFFE

SEQ ID NO: 30 - protein, 311 aa, Escherichia coli
MVLLAGAALSIAPVQAASYPTKQIELVVPYAAGGGTDLVARAFADAAKNHLPVSIGVINKPGGGGAIGLSEIAAARPNGYKIGLGTVELTTLPSLGMVRFKTSDFKPIARLNADPAAITVRADAPWNSYEEFMAYSKANPGKVRIGNSGTGAIWHLAAAALEDKTGTKFSHVPYDGAAPAITGLLGGHIEAVSVSPGEVINHVNGGKLKTLVVMADERMKTMPDVPTLKEKGVDLSIGTWRGLIVSQKTPQDVVDVLAKAAKETAEEPAFQDALQKLNLNYAWLDAASFQTQISEQEKYFDELLTRLGLKK

SEQ ID NO: 31 - protein, 722 aa, Escherichia coli
MLRWKRCIILTFISGAAFAAPEINVKQNESLPDLGSQAAQQDEQTNKGKSLKERGADYVINSATQGFENLTPEALESQARSYLQSQITSTAQSYIEDTLSPYGKVRLNLSIGQGGDLDGSSIDYFVPWYDNQTTVYFSQFSAQRKEDRTIGNIGLGVRYNFDKYLLGGNIFYDYDFTRGHRRLGLGAEAWTDYLKFSGNYYHPLSDWKDSEDFDFYEERPARGWDIRAEVWLPSYPQLGGKIVFEQYYGDEVALFGTDNLEKDPYAVTLGLNYQPVPLLTVGTDYKAGTGDNSDVSINATLNYQFGVPLKDQLDSDKVKAAHSLMGSRLDFVERNNFIVLEYKEKDPLDVTLWLKADATNEHPECVIKDTPEAAVGLEKCKWTINALINHHYKIVAASWQAKNNAARTLVMPVIKENTLTEGNNNHWNLVLPAWQYSSDQAEQEKLNTWRVRLALEDEKGNRQNSGVVEITVQQDRKIELIVNNIANPEENNHSHEASAQADGVDGVVMDLDVTDSFGDNTDRNGDALPEDNLTPQLYDAQDKRVTLTNKPCSTDNPCVFIAKQDKEKGTVTLSSTLPGTYRWKAKAAPYDDSNYVDVTFLGAEIGGLNAFIYRVGAAKPSNLIGKDKEPLPSTTFIDLFYGATTIKTVSSSRSKNLTKRWCSTTTSGNLPARASMVSGCTGEHSNEDIVIPATNREAAQTYGAQAGDGLQGYGLRVLYTKK

SEQ ID NO: 32 - protein, 319 aa, Escherichia coli
MKQDKRRGLTRIALALALAGYCVAPVALAEDSAWVDSGETNIFQGTIPWLYSEGGSATTDADRVTLTSDLKGARPQGMKRTSVFTRVINIGDTEGDVDLGGLGDNAKTIDTIRWMSYKDAQGGDPKELATKVTSYTLTDADRGRYIGIEITPTTQTGTPNVGTALHLYDVSTASGGGSDSDNVAPGPVVNQNLKVAIFVDGTSINLINGSTPIELGKTYVAKLYSDENKNGKFDAGTDADVTANYDFRWVLSGSSQQLGTSGGIVNSSFDNNNLVIPATNDEARTNLNGPARDGKEALSIPTNGDGVQGYKLHIIYKHK

SEQ ID NO: 33 - protein, 629 aa, Escherichia coli
MKKVLTLSLLALCVSHSAVAANYTFNNDNIALSFDDTNSTIVLKDRRTNHPITPQELFFLTLPDETKIHTADFKIKHIKKQDNAIVIDFTRPDFNVTVQLNLVKGKYASIDYTIAAVGQPRDVAKITFFPTKKQFQAPYVDGAITSSPIIADSFFILPNKPIVNTYAYEATTNLNVELKTPIQPETPVSFTTWFGTFPETSQLRRSVNQFINAVRPRPYKPYLHYNSWMDIGFFTPYTEQDVLGRMDEWNKEFISGRGVALDAFLLDDGWDDLTGRWLFGPAFSNGFSKVREKADSLHSSVGLWLSPWGGYNKPQRRSRFACKRVWVRNRGRQAGAFGSELLKNFNEQIINLIKNEHITSFKLDGMGNASSHIKGSPFASDFDASIALLHNMRRANPNLFINLTTGTNASPSWLFYADSIWRQGDDINLYGPGTPVQQWITYRDAETYRSIVRKGPLFPLNSLMYHGIVSAENAYYGLEKVQTDSDFADQVWSYFATGTQLQELYITPSMLNKVKWDTLAKAAKWSKENASVLVDTHWIGGDPTALAVYGWASWSKDKAILGLRNPSDKPQTYYLDLAKDFEIPAGNAAQFSLKAVYGSNKTVPVEYKNATVITLQPLETLVFEAVTIN

SEQ ID NO: 34 - protein, 1778 aa, Escherichia coli
MNKIFKVIWNPATGSYTVASETAKSRGKKSGRSKLLISALVAGGLLSSFGASADNYTGQPTDYGDGSAGDGWVAIGKGAKANTFMNTSGASTALGYDAIAEGEYSSAIGSKTLATGGASMAFGVSAKAMGDRSVALGASSVANGDRSMAFGRYAKTNGFTSLAIGDSSLADGEKTIALGNTAKAYEIMSIALGDNANASKEYAMALGASSKAGGADSLAFGRKSTANSTGSLAIGADSSSSNDNAIAIGNKTQALGVNSMALGNASQASGESSIALGNTSEASEQNAIALGQGSIASKVNSIALGSNSLSSGENAIALGEGSAAGGSNSLAFGSQSRANGNDSVAIGVGAAAATDNSVAIGAGSTTDASNTVSVGNSATKRKIVNMAAGAISNTSTDAINGSQLYTISDSVAKRLGGGATVGSDGTVTAVSYALRSGTYNNVGDALSGIDNNTLQWNKTAGAFSANHGANATNKITNVAKGTVSATSTDVVNGSQLYDLQQDALLWNGTAFSAAHGTEATSKITNVTAGNLTAGSTDAVNGSQLKTTNDNVTTNTTNIATNTTNITNLTDAVNGLGDDSLLWNKAAGAFSAAHGTEATSKITNVTAGNLTAGSTDAVNGSQLKTTNDNVTTNTTNIATNTTNITNLTDAVNGLGDDSLLWNKTAGAFSAAHGTDATSKITNVTAGNLTAGSTDAVNGSQLKTTNDNVTTNTTNIATNTTNITNLTDAVNGLGDDSLLWNKTAGAFSAAHGTDATSKITNVKAGDLTAGSTDAVNGSQLKTTNDNVSTNTTNITNLTDAVNGLGDDSLLWNKTAGAFSAAHGTDATSKITNVKAGDLTAGSTDAVNGSQLKTTNDNVSTNTTNITNLTDSVGDLKDDSLLWNKAAGAFSAAHGTEATSKITNLLAGKISSNSTDAINGSQLYGVADSFTSYLGGGADISDTGVLSGPTYTIGGTDYTNVGDALAAINTSFSTSLGDALLWDATAGKFSAKHGINNAPSVITDVANGAVSSTSSDAINGSQLYGVSDYIADALGGNAVVNTDGSITTPTYAIAGGSYNNVGDALEAIDTTLDDALLWDTTANGGNGAFSAAHGKDKTASVITNVANGAVSATSNDAINGSQLYSTNKYIADALGGDAEVNADGTITAPTYTIANTDYNNVGEALDALDNNALLWDEDAGAYNASHDGNASKITNVAAGDLSTTSTDAVNGSQLNATNILVTQNSQMINQLAGNTSETYIEENGAGINYVRTNDSGLAFNDASASGIGATAVGYNAVASHASSVAIGQDSISEVDTGIALGSSSVSSRVIVKGTRNTSVSEEGVVIGYDTTDGELLGALSIGDDGKYRQIINVADGSEAHDAVTVRQLQNAIGAVATTPTKYYHANSTAEDSLAVGEDSLAMGAKTIVNGNAGIGIGLNTLVLADAINGIAIGSNARANHADSIAMGNGSQTTRGAQTNYTAYNMDAPQNSVGEFSVGSEDGQRQITNVAAGSADTDAVNVGQLKVTDAQVSQNTQSITNLNTQVTNLDTRVTNIENGIGDIVTTGSTKYFKTNTDGADANAQGKDSVAIGSGSIAAADNSVALGTGSVADEENTISVGSSTNQRRITNVAAGVNATDAVNVSQLKSSEAGGVRYDTKADGSIDYSNITLGGGNSGTTRISNVSAGVNNNDAVNYAQLKQSVQETKQYTDQRMVEMDNKLSKTESKLSGGIASAMAMTGLPQAYTPGASMASIGGGTYNGESAVALGVSMVSANGRWVYKLQGSTNSQGEYSAALGAGIQW

SEQ ID NO: 35 - protein, 227 aa, Escherichia coli
MNLKKTLLSVLMILQLCLLVGCDYIEKASKVDDLVTQQELQKSKIEALEKQQELDKRKIEHFEKQQTTIINSTKTLAGVVKAVKNKQDEFVFTEFNPAQTQYFILNNGSVGLAGKILSIDAVENGSVIRISLVNLLSVPVSNMGFYATWGGEKPTDINALAKWQQLLFSTAMNSSLKLLPGQWQDINLTLKGVSPNNLKYLKLAINMANIQFDRLQPAESPQRKNKK

SEQ ID NO: 36 - protein, 1109 aa, Escherichia coli
MKRVVRLLGVGLLLLVVLLLILFVLAQTTPLISAQDEHAVWLRLLITAIVICLLSMCIFFLFSFRQNEASTISLYAQPTDIKEINTEQPNYASLLTIYLRDRYGPFWRRKVRLLLVTGEPEQAEAIAPGLTGQHWLEGDHTVLIYGGRPTAEPDVTLLTALKKLRRSRPLDGIIWALTEEQSRQTAQLDKGWRGLINGGKRLGFQAPLYLWQVCDDGDYQTGRPLQSVGCLLPERCTPEQLAVMLEAAADGTGHVAATDRYRMFSAASGSYPCRAGYCSLADRPETAAGRRRIFFPAPARPDVQPAACRRAGGQHLMQWLPSPVWAGVTVITRAGARWVFLWLRTALMSAVCVLVIWGAGMTTSFFANRALVQETGIQTARALDTRLPLAEQLVALHTLQGELERLQYRIREGAPWYQRFGLERNQQLLAAAFPGYAQAANRLVRDVAVDHLQQQLNAFVALPPNSPQRTATGEQRYKQLKALLMTSRPEKADAAFFSTTLMADGLRYENIPEGVRQSVLPSLLTFWTANLPEHPQWKTSPPPELTGAVRKILLRQIGVRNAENTLYQNVLQQVSRNYADMTLADMTGDTLTESLFSTEQTVPGMFTRQAWEGQVREAIEQVVTARREEIDWVLSDRQQDTSADISPDTLRNRLTSRYFTDFAGSWLAFLNSIHWKKEDSLSGILDQLTLMADARQSPLIALTDTLAWQAATGRENRGLSDSLAKSAQELFNGKEKTPQQSREGDDVPVGPLDKTFTPLLRLLGDKAGGGDSQLSLQTYLTRVTRVRLKLQQVTNAPDPQEMTQQLAQTVLQGKTVDLTDTRDYGRLIAASLGEEWSGFGQALFVRPVEQSWRQVLTPAADSLNRQWQRAIVSHWNQDFAGRYPFKASQNDASLPLLAQYLRDDGRINLFIAANLSGVLKREGRYWVADAMNTQGLTVNPDFIRALNRLRDVADTAFASGDAGIHFELRAKPARDVMKTHLVIDGQELEYFNQKERWQRFNWPDEQWQPGASLSWTSTQAMERILADYRGSWSLIRLLEQAQVTPVDSSTFKVVWKAQDGLPLNYLLRVEQGKGPLALLELKNFRLPGQVFLTGKSMKDVEEYGEDADE

SEQ ID NO: 37 - protein, 178 aa, Escherichia coli
MFPIRFKRPALLCMAMLTVVLSGCGLIQKVVDESKSVASAVFYKQIKILHLDFFSRSALNTDAEDTPLSTMVHVWQLKTREDFDKADYDTLFMQEEKTLEKDVLAKHTVWVKPEGTASLNVPLDKETQFVAIIGQFYHPDEKSDSWRLVIKRDELEADKPRSIELMRSDLRLLPLKDK

SEQ ID NO: 38 - protein, 280 aa, Escherichia coli
MISGGNMLKEWMIFTCSLLTLAGASLPLSGCISRGQESISEGAAFGAGILREPGATKKADTKDLNVPPPVYGPPQVIFRIDDNRYFTLENYTHCENGQTFYNNKAKNIHVKILDASGYLFKGRLFWLSTRDDFLAFPATLNTRHASCMGSNKGCMNAVIVTTDGGKRRSGVPYGSYTQNPTGATRDYDMLVMNDGFYLLRYRGGQGRFSPVILRWILSTEDSSGVVRSEDAYELFRPGEEVPSTGFYKIDLSRFYPKNNVMEMQCDRTLEPVQPSESKIQ

SEQ ID NO: 39 - protein, 501 aa, Escherichia coli
MEHVSIKTLYHLLCCMLLFISAMCALAQEHEPIGAQDERLSTLIHQRMQEAKVPALSVSVTIKGVRQRFVYGVADVASQKANTLDTVYELGSMSKAFTGLVVQILIQEGRLRQGDDIITYLPEMRLNYQGKPASLTVADFLYHTSGLPFSTLARLENPMPGSAVAQQLRNENLLFAPGAKFSYASANYDVLGAVIENVTGKTFTEVIAERLTQPLGMSATVAVKGDEIIVNKASGYKLGFGKPVLFHAPLARNHVPAAYIHSTLPDMEIWIDAWLHRKALPATLREAMSNSWRGNSDVPLAADNRILYASGWFIDQNQGPYISHGGQNPNFSSCIALRPDQQIGIVALANMNSNLILQLCADIDNYLRIGKYADGAGDAITATDTLFVYLTLLLCFWGAVVVVRGAFRVYRATAHGPGKQQRLRLRVRDYIIALAVPGLVAAMLYVAPGILSPGLDWRFILVWGPSSVLAIPFGIILLAFVLTLNHQIKRILLHNKEWDDE

SEQ ID NO: 40 - protein, 682 aa, Escherichia coli
MKNKYIIAPGIAVMCSAVISSGYASSDKKEDTLVVTASGFTQQLRNAPASVSVITSEQLQKKPVSDLVDAVKDVEGISITGGNEKPDISIRGLSGDYTLILVDGRRQSGRESRPNGSGGFEAGFIPPVEAIERIEVIRGPMSSLYGSDAIGGVINIITKPVNNQTWDGVLGLGGIIQEHGKFGNSTTNDFYLSGPLIKDKLGLQLYGGMNYRKEDSISQGTPAKDNKNITATLQFTPTESQKFVFEYGKNNQVHTLTPGESLDAWTMRGNLKQPNSKRETHNSRSHWVAAWNAQGEILHPEIAVYQEKVIREVKSGKKDKYNHWDLNYESRKPEITNTIIDAKVTAFLPENVLTIGGQFQHAELRDDSATGKKTTETQSVSIKQKAVFIENEYAATDSLALTGGLRLDNHEIYGSYWNPRLYAVYNLTDNLTLKGGIAKAFRAPSIREVSPGFGTLTQGGASIMYGNRDLKPETSVTEEIGIIYSNDSGFSASATLFNTDFKNKLTSYDIGTKDPVTGLNTFIYDNVGEANIRGVELATQIPVYDKWHVSANYTFTDSRRKSDDESLNGKSLKGEPLERTPRHAANAKLEWDYTQDITFYSSLNYTGKQIWAAQRNGAKVPRVRNGFTSMDIGLNYQILPDTLINFAVLNVTDRKSEDIDTIDGNWQVDEGRRYWANVRVSF

SEQ ID NO: 41 - protein, 164 aa, Escherichia coli
MGFRKTIITSVGLIFISFSFVAKCSQLKNLNNYSVMLCGKVSNNILDDIGGYKERNILMLRAIKKIIIMTIVNIIFFYSFQSTADEMVLIKKYGFGLERDIKGRPLIYPIENYDECKKKCNHMNYIADVNAQLAMSKKNNRIFANITFTNNSSTTYFFLNIIYL

SEQ ID NO: 42 - protein, 218 aa, Escherichia coli
MNQIKDNKVIMKIKNLISVILLSGGIMGTGLYSSDNHQKIRSRFNIQESYCAIKTNGVLGFSNRKDVLRENGDSTGTTSSSTNAMMLMENGENEISLEIGALRWFSDKPASTEERGHFSQKAGCSLDLVRFVKQEETILSSIKVTINQQGIPEAQPDSMHPVIRKEILAEQAEPGFIDPDYFNETYFPKGMKVYQFTQKVSVAGLPDGPGRSTPFTGA

SEQ ID NO: 43 - protein, 2732 aa, Escherichia coli
MHQPPVRFTYRLLSYLVSAIIAGQPLLPAVGAVITPQNGAGMDKAANGVPVVNIATPNGAGISHNRFTDYNVGKEGLILNNATGKLNPTQLGGLIQNNPNLKAGGEAKGIINEVTGGKRSLLQGYTEVAGKAANVMVANPYGITCDGCGFINTPHATLTTGKPVMNADGSLQALEVTEGSITINGAGLDGTRSDAVSIIARATEVNAALHAKDLTVTAGANRVTADGRVRALKGEGDVPKVAVDTGALGGMYARRIHLTSTESGVGVNLGNLYARDGDITLDASGRLTVNNSLATGAVTAKGQGVTLTGDHKAGGNLSVSSRRDIVLSNGTLNSDKDLSLTAGGRITQQNEKLTAGRDVTLAAKNITQDTASQINAARDIVTVASDTLTTQGQITAGQNLTASATTLTQDGILLAKSHAGLNAGTLNNSGAVQGATLTLGSTTLSNSGSLLSGGPLTMNTRDFTQSGRTGAKGKVDIMASGKLTSTGLLVTMHLVLKAQDVTQNGVLSGGKGLTVSATSSGKKSVTHSDAAMTLNVTTVALDGETSAGDTLRVQADKLSTAAGAQLQSGKNLSINARDARLAGTQAAQQTMVVNASEKLTHSGKSSAPSLSLSAPELTSSGVLVGSALNTQSQTLTNSGLLQGEASLTVNTQRLDNQQNGTLYSAADLTLDIPDIRNSGLITGDNGLMLNAVSLSNPGKIIADTLSVRATTLDGDGLLQGAGALALAGDTLSQGSHGRWLTADDLSLRGKTLNTAGTTQGQNITVQADRWANSGSVLATGNLTASATGQLTSTGDIMSQGDTTLKAATTDNRGSLLSAGTLSLDGNSLDNRGTVQGNHVTIRQNSVTNSGTLTGIAALTLAARMASPQPALMNNGGSLLTSGDLTITAGSITSSGHWQGKRVLITADSLANSGAIQAADSLTARLTGELVSTAGSKVTSNGEMALSALNLSNSGQWIAKNLTLKANSLTSAGDITGVDTLTLTVNQTLNNQANGKLLSAGVLTLKADSVTNDGQLQGNVTTITAGQLTNGGHLQGETLTLTASGGVNNRSGGVLMSRNALNVSTATLSNQSTIQGGGGVSLNATDRLQNDGKILSGSNLTLTAQVLANTGSGLVQAATLLLDVVNTVNGGRVLATGSDVKGTTLNNTGTLQGATLVNYHTFSSGTLLGTSGLGVKGSSLLQNGTGRLYSAGNLLLDAQDFSGQGQVVATGDVTLKLIAALTNHGTLAAGKTLSVTSQNAITNGGVMQGDAMVLGAGEAFTNNGLTAGKGNSVFSAQRLFLNAPGSLQGGGDVSLNSRSDITISGFTGTAGSLTMNVAGTLLNSALIYAGNNLKLFTDRLHNQHGDILAGNSLWVQKDASGGANTEIINNSGNIETHQGDIVVRTGHLLNQREGFSATTTTRTNPSSIQGMGNALVDIPLSLLPDGSYGYFTREVENQHGTPCNGHGACNITMDTLYYYAPFADSATQRFLSSQNITTVTGADNPAGRIASGRNLSAEAERLENRASFILANGDIALSGRELSNQSWQTGTENEYLVYRYDPKTFYGSYATGSLDKLPLLSPEFENNTIRFSLDGREKDYTPGKTYYSVIQAGGDVKTRFTSSINNGTTTAHAGSVSPVVSAPVLNTLSQQTGGDSLTQTALQQYEPVVVGSPQWHDELAGALKNIAGGSPLTGQTGISDDWPLPSGNNGYLVPSTDPDSPYLITVNPKLDGLGQVDSHLFAGLYELLGAKPGQAPRETAPSYTDEKQFLGSSYFLDRLGLKPEKDYRFLGDAVFDTRYVSNAVLSRTGSRYLNGLGSDTEQMRYLMDNAARQQKGLGLEFGVALTAEQIAQLDGSILWWESVTINGQTVMVPKLYLSPEDITLHNGSVISGNNVQLAGGNITNSGGSINAQNDLSLDSSGYIDNLNAGLISAGGSLDLSAIGDISNISSVISGKTVQLESVSGNISNITRRQQWNAGSDSQYGGVHLSGTDTGPVATIKGTDSLSLDAGKNIDITGATVSSGGDLGMSAGNDINIAANLISGSKSQSGFWHTDDNSSSSTTSQGSSISAGGNLAMAAGHNLDVTASSVSAGHSALLSCRSRPSLECSQGKAKTSRNGRSESHESHAAVSTVTAGDNFLLVAGRDIASQAAGMAAENNVVIRGGRDVNLVAESAGAGDSYTSKKKKEINETVRQQGTEIASGGDTTVNAGRDITAVASSVTATGNISVNAGRDVALTTATESDYHYLETKKKSGGFLSKKTTRTISEDSATREAGSLLSGNRVTVNAGDNLTVEGSDVVADRDVSLAAGNHVDVLAATSTDTSWRFKETKKSGLMGTGGIGFTIGSSKTTHDRREAGTTQSQSASTIGSTAGNVSITAGKQAHISGSDVIANRDISITGDSVVVDPGHDRRTVDEKFEQKKSGLTVALSGTVGSAINNAVTSAQETKESSDSRLKALQATKTALSGVQAGQAATMASATGDPNAGVSLSLTTQKSKSQQHSESDTVSGSTLNAGNNLSVVATGKNRGDNRGDIVIAGSQLKAGGNTSLDAANDILLSGAANTQKTTGRNSSSGGGVGVSIGAGKGAGISAFASVNAAKGREKGNGTTTDKTVTINSGRDTVLNGAQVNGNRIIADVGHDLLISSQQDTSKYDSKQTSVAAGGSFTFGSMTGSGYIAASRDKMKSRFDSVAEQTGMFARVMVASTSQWVNIPNWMVRSLPHCHTGEKPPGYRTLGLVTLQRSGIIKSSHRWNQS

SEQ ID NO: 44 - protein, 321 aa, Escherichia coli
MMLKKTIFILTLFSGNVIAATVELGFENEQYNYAYRSADVFMPYIKSNFNPVTDSALNVSLTYMYQDQYGKKHKKTSEDRFKTNRDRIELYLKGYTLNRGAYSFSPSAGFRYESWDVNYDNPKKQDKWKLELRFYPNMTYKLNDQLSLYMNGFVAPVFFKTQQESRKDNNYVKGKLGAKRYNNDYYQELQILGVRYKFNNDNTLWASVYNERKYNQHSSKYDRWQLRGGYDFKVTEEFVLSPFIRYDLSYREKNLESTSNNGLSKNNKEIRTGASFSYKIIPSVKLVGEIYRQTTNIENYYGEHSEDKNRMFYKLGINKTF

SEQ ID NO: 45 - protein, 587 aa, Escherichia coli
MQHRQKNILTKTSLLSRALSVPCCDMFRRGSPWICYLSLSVFSGCFIPAFSSPAAMLSPGDRSAIQQQQQQLLDENQRQRDALERPLTITPSPETSAGTEGPCFTVSSIVVSGATRLTSAETDRLVPWVNQCLNITGLTAVTDAVTDGYIRRGYITSRAFLTEQDLSGGVLHITVMEGRLQQIRAEGADLPARTLKMVFPGMEGKVLNLRDIEQGMEQINRLRTEPVQIEISPGDREGWSVVTLTALPEWPVTGSVGIDNSGQKSTGTGQLNGVLSFNNPLGLADNWFVSGGRSSDFSVSHDARNFAAGVSLPYGYTLVDYTYSWSDYLSTIDNRGWRWRSTGDLQTHRLGLSHVLFRNGDMKTALTGGLQHRIIHNYLDDVLLQGSSRKLTSFSVGLNHTHKFLGGVGTLNPVFTRGMPWFGAESDHGKRGDLPVNQFRKWSVSASFQRPVTDRVWWLTSAYAQWSPDRLHGVEQLSLGGESSVRGFKDQYISGNNGGYLRNELSWSLFSLPYVGTVRAVAALDGGWLHSDSDDPYSSGTLWGAAAGLSTTSGHVSGSFTAGLPLVYPDWLAPDHLTVYWRVAVAF

SEQ ID NO: 46 - protein, 744 aa, Escherichia coli
MNKHTLLLTVLFLNLICTPVFAQNWQVATFGQSTDLNFSSLIDSAKIGRNNAWLAGNNNFLEAGKFYTLPTDFFIESRGGKIANSHDGMTVFYTIVPVTQTFRLEADLTLEQIGPEVNGKSPAGQEGAGLFVRDIIGPQRQEPQSAGTEEYPQASNILMNAFITQNKKNDNLVQITSIVREGVIKTWGNEGITIKKQPIIENINFTQKRNIHMTIERLPEKFILTAFDTDRKENQSWQFSDYSGFMNQLDNNSLAIGFFAARNAKLRVKNASFKPGKPLVDYKQLTSRQFSRVRHKAPELFLASPQSVVRNSTTLQFLANQAGIVSIDNDKQTKQVQAGELVQFPVTLQKKHNDFTVNFNVDGNISKKAIRIEQVKSNLTDPYEIYVCSDCRQGARGSKNDPVDLQTAVKFVAPGGNIYLNDGQYHGITLDRELSGIPGKYKTISAINPHKAIFINKTFNLDASYWHLKSVVFDGNVDNGNNKPAYLRIAGSYNIIEHVIARNNDDTGISISAKDKNRFFWPAHNLVLNSDSYNNLDLSGINADGFAAKLGVGPGNIFRGCIAHNNADDGWDLFNKIEDGPNASVTIENSVAYENGLPYNKADILKGSIGNGGEGQPSKSQVINSIAINNNMDGFTDNFNTGSLIVRNNIAMNNARYNYILRTNPYKFPSSILFDNNYSIRDDWENKIKDFLGDTVNSVNYKLLVSHETGPVQKDLFFTRDDSGNIIYPDFFLNIINKFNZTMP

SEQ ID NO: 47 - protein, 136 aa, Escherichia coli
MKTFIKTLLVAVTILFSVFATAKQVKLPNNIKYVNTTEAFSCTEIDGMNCQTKNPFNYKDNSYVFVLERGGAWCYDYTVSVLNLKTGKAQMLEYKDNQLCSGSNKPFFEIKNGVPTVGVIDTSGKPVVVALDKLKT

SEQ ID NO: 48 - protein, 225 aa, Escherichia coli
MQLPVKLLMSLISLVSVIARAGKYKNYIRDEIKYWRYTSYKGGEFPEGFTDEKFSSAIYNGRIFTMKRLHTLMLFLAVLFTGFNVEAASVKQALSCDPNARAEQPGACPTTYELYEGDAAYKAALDKALKPVGLSGMFGKGGYMDGPGGNVTPVTINGTVWLQGDGCKANTCGWDFIVTLYNPKTHEVVGYRYFGLDDPAYLVWFGEIGVHEFAYLVKNYVAAVN

SEQ ID NO: 49 - protein, 721 aa, Escherichia coli
MKTQITFAALLPALASFIPLHAHASSTSEDEMIVTGNTAADTTDSAAGAGFKTNDIDVGPLGTKSWIETPYSSTTVTKEMIENQQAQSVSEMLKYSPSTQMQARGGMDVGRPQSRGMQGSVVANSRLDGLNIVSTTAFPVEMLERMDVLNSLTGALYGPASPAGQFNFVAKRPTEETLRKVTLGYQSRSAFTGHADLGGHFDENKRFGYRVNLLDQEGEGNVDDSTLRRKLVSVALDWNIQPGTQLQLDASHYEFIQKGYVGSFNYGPNVKLPSAPNPKDKNLALSTAGNDLTTDTISTRLIHYFNDDWSMNAGVGWQQADRAMRSVSSKILNNQGDISRSMKDSTAAGRFRVLSNTAGLNGHIDTGSIGHDLSLSTTGYVWSLYSAKGTGSSYSWGTTNMYHPDAIDEQGDGKIRTGGPRYRSSVNTQQSVTLGDTVTFTPQWSAMFYLSQSWLQTKNYDKHGNQTNQVDENGLSPNAALMYKITPNTMAYVSYADSLEQGGTAPTDESVKNAGQTLNPYRSKQYEVGLKSDIGEMNLGAALFRLERPFAYLDTDNVYKEQGNQVNNGLELTAAGNVWQGLNIYSGVTFLDPKLKDTANASTSNKQVVGVPKVQANLLAEYSLPSIPEWVYSANVHYTGKRAANDTNTSYASSYTTWDLGTRYTTKVSNVPTTFRVVVNNVFDKHYWASIFPSGTDGDNGSPSAFIGGGREVRASVTFDF

SEQ ID NO: 50 - protein, 669 aa, Escherichia coli
MKNITLWQRLRQVSISTSLRCAFLMGALLTLIVSSVSLYSWHEQSSQIRYSLDKYFPRIHSAFLIEGNLNLVVDQLNEFLQAPNTTVRLQLRTQIIQHLDTIERLSRGLSSRERQQLTVILQDSRSLLSELDRALYNMFLLREKVSELSARIDWLHDDFTTELNSLVQDFTWQQGTLLDQIASRQGDTAQYLKRSREVQNEQQQVYTLARIENQIVDDLRDRLNELKSGRDDDIQVETHLRYFENLKKTADENIRMLDDWPGTITLRQTIDELLDMGIVKNKMPDTMREYVAAQKALEDASRTREATQGRFRTLLEAQLGSTHQQMQMFNQRMEQIVHVSGGLILVATALALLLAWVFNHYFIRSRLVKRFTLLNQAVVQIGLGGTETTIPVYGNDELGRIAGLLRHTLGQLNVQKQQLEQEITDRKVIEADLRATQDELIQTAKLAVVGQTMTTLAHEINQPLNALSMYLFTARRAIEQTQKEQASMMLGKAEGVISRIDAIIRSLRQFTRRAELETSLHAVDLAQMFSAAWELLAMRHRSLQATLVLPQGTATVSGDEVRTQQVLVNVLANALDVCGQGAVITVNWQMQGKTLNVFIGDNGPGWPEALLPSLLKPFTTSKEVGLGIGLSICVSLMEQMKGELRLASTMTRNACVVLQFRLTDVEDAK

SEQ ID NO: 51 - protein, 753 aa, Escherichia coli
MNVIKLAIGSGILLLSCGAYSQSISEKTNSDKKGAAEFSPLSVSVGKTTSEQEALEKTGATSSRTTDKNLQSLDATVRSMPGTYTQIDPGQGAISVNIRGMSGFGRVNTMVDGITQSFYGTSTSGTTTHGSTNNMAGVLIDPNLLVAVDVTRGDSSGSEGINALAGSANMRTIGVDDVIFNGNTYGLRSRFSVGSNGLGRSGMIALGGKSDAFTDTGSIGVMAAVSGSSVYSNFSNGSGINSKEFGYDKYMKQNPKSQLYKMDIRPDEFNSFELSARTYENKFTRRDITSDDYYIKYHYTPFSELIDFNVTASTSRGNQKYRDGSLYTFYKTSAQNRSDALDINNTSRFTVADNDLEFMLGSKLMRTRYDRTIHSAAGDPKANQESIENNPFAPSGQQDISALYTGLKVTRGIWEADFNLNYTRNRITGYKPACDSRVICVPQGSYDIDDKEGGFNPSVQLSAQVTPWLQPFIGYSKSMRAPNIQEMFFSNSGGASMNPFLKPERAETWQAGFNIDTRDLLVEQDALRFKALAYRSRIQNYIYSESYLVCSGGRKCSLPEVIGNGWEGISDEYSDNMYIYVNSASDVIAKGFELEMDYDAGFAFGRLSFSQQQTDQPTSIASTHFGAGDITELPRKYMTLDTGVRFFDNALTLGTIIKYTGKARRLSPDFEQDEHTGAIIKQDLPQIPTIIDLYGTYEYNRNLTLKLSVQNLMNRDYSEALNKLNMMPGLGDETHPANSARGRTWIFGGDIRF

SEQ ID NO: 52 - protein, 133 aa, Escherichia coli
MSSKTKCWLWMLLVILSETSATSTLKMFDNSEGMTKTLLLALIVVLYCICYYSLSRAVKDIPVGLAYATWSGTGILMVSTLGILFYGQHPDTAAIIGMVIIASGIIIMNLFSKMGSEEAEETPVTNLDKKIAN

SEQ ID NO: 53 - protein, 286 aa, Escherichia coli
MYIKKHWIALSILLIPCIGNAQEIKIDESWLHQSLNVIGRTDSRFGPRLTNDLYPEYTVAGRKDWFDFYGYVDLPKFFGVGSHYDVGIWDEGSPLFTEIEPRFSIDKLTGLNLAFGPFKEWFIANNYVYDMGDNQSSRQSTWYMGLGTDIDTGLPIKLSANIYAKYQWQNYGAANENEWDGYRFKIKYSIPLTNLFGGRLVYNSFTNFDFGSDLADKSHNNKRTSNAIASSHILSLLYEHWKFAFTLRYFHNGGQWNAGEKVNFGDGPFELKNTGWGTYTTIGYQF

SEQ ID NO: 54 - protein, 172 aa, Escherichia coli
MRIAPRTFFAISALAFIVASGFSFWRLSPAENTGIMSCSTKGIMRFENMEKENVNGNIHFNFGSQGKGSMVLEGYTDSAAGWLYLQRYVKFTYTSKRVSATERHYRISQWESSASSIDESPDVIFDYFMREMSDSHDGLFLNAQKLNDKAILLSSINSPLWICTLKSGSKLD

SEQ ID NO: 55 - protein, 182 aa, Escherichia coli
MKIKVIALATFVSAVFAGSAMAYDGTITFTGKVVAQTCTVNTSDKDLAVTLPTVATSSLKDNAATSGLTPFAIRLTGCATGMNSAQNVKAYFEPSSNIDLATHNLKNTATPTKADNVQIQLLNSNGTSTILLGEADNGQDVQSETIGSDGSATLRYMAQYYATGQSTAGDVKATVHYTIAYE

SEQ ID NO: 56 - protein, 359 aa, Escherichia coli
MKRIFFIPLFLILLPKLAVAGPDDYVPSQIAVNTSTLPGVVIGPADAHTYPRVIGELAGTSNQYVFNGGAIALMRGKFTPALPKIGSITVYFPSRKQRDSSDFDIYDIGVSGLGIIIGMAGYWPATPLVPINSSGIYIDPVGANTNPNTYNGATASFGARLFVAFVATGRLPNGYITIPTRQLGTILLEAKRTSLNNKGLTAPVMLNGGRIQVQSQTCTMGQKNYVVPLNTVYQSQFTSLYKEIQGGKIDIHLQCPDGIDVYATLTDASQPVNRTDILTLSSESTAKGFGIRLYKDSDVTAISYGEDSPVKGNGSQWHFSDYRGEVNPHINLRANYIKIADATTPGSVKAIATITFSYQ

SEQ ID NO: 57 - protein, 844 aa, Escherichia coli
MNANNLSCLIYCRCSLLLFAALGLTVTNHSFAAEEAEFDSEFLHLDKGINAIDIRRFSHGNPVPEGRYYSDIYVNNVWKGKADLQYLRTANTGAPTLCLTPELLSLIDLVKDTMSGNTSCFPASTGLSSARINFDLSTLRLNIEIPQALLNTRPRGYISPAQWQSGVPAAFINYDANYYQYSSSGTSNEQTYLGLKAGFNLWGWALRHRGSESWNNSYPAGYQNIETSIMHDLAPLRAQFTLGDFYTNGELMDSLSLRGVRLASDERMLPGSLRGYAPAVRGIANSNAKVTIYQNAHILYETTVPAGPFVINDLYPSGYAGDLLVKITESNGQTRMFTVPFAAVAQLIRPGFSRWQMSVGKYRYANKTYNDLIAQGTYQYGLTNDITLNSGLTTASGYTAGLAGLAFNTPLGAIASDITLSRTAFRYSGVTRKGYSLHSSYSINIPASNTNITLAAYRYSSKDFYHLKDALSANHNAFIDDVSVKSTAFYRPRNQFQISINQELGEKWGGMYLTGTTYNYWGHKGSRNEYQIGYSNFWKQLGYQIGLSQSRDNEQQRRDDRFYINFTLPLGGSVQSPVFSTVLNYSKEEKNSIQTSISGTGGEDNQFSYGISGNSQENGPSGYAMNGGYRSPYVNITTTVGHDTQNNNQRSFGASGAVVAHPYGVTLSNDLSDTFAIIHAEGAQGAVINNASGSRLDFWGNGVVPYVTPYEKNQISIDPSNLDLNVELSATEQEIIPRANSATLVKFDTKTGRSLLFDIRMSTGNPPPMASEVLDEHGQLAGYVAQAGKVFTRGLPEKGHLSVVWGPDNKDRCSFVYHVAHNKDDMQSQLVPVLCIQHPNQEKT

SEQ ID NO: 58 - protein, 277 aa, Escherichia coli
MVKCHTLINRRNKCLLIVFIVLIGWIIFRPKAYTYSLNDKEKEMLIMLSQHPETRYFGFYSIELPADYKPTGMVMFIQGSAMIPVETKLQYYPPFLQYMTRYEAELKNTSALDPLDTPYLKQVHPLSPPMNGVIFERMKAKYTPDFARVLDAWKWENGVTFSVKIEAKDGRATRYDGISKIAEYSYGYNIPEKKVQLLTILSGLQPRADNQPPSENKLAIQYAQVDASLLGEYELSVDYKNSNNIKISLQTDNNSYIDSLLDIRYPSNGNRAWYNSI

SEQ ID NO: 59 - protein, 366 aa, Escherichia coli
MLPEPVYRRWIILLISMLTVGTLFILSVWNSATYWDIFIYGVLPMLFLWLCLFGIALNKYEQSVAACISWESERQQVKQLWQHWSQKQLAIVGNVLFTPEEKGMSVLLGPQEEIPAYPKKARPLFSASRYSLSSIFHDIHQQLTQQFPDYRHYLHTIYVLQPEKWRGETVRQAIFHQWDLVPERTNTLNQIQSLYDERFDGLILVVCLQNWPENKPEDTSELVSAQLISSSSFVRQHQIPVIAGLGRVMPLEPEELEHNLDVLFEYNQLDNKQLQHVWVSGLDEGTIENLMQYAEQHQWSLPKKRPLHMIDHSFGPTGEFIFPVSLAMLSEAAKETEQNHLIIYQSAQYAQKKSLCLITRKLYLRT

SEQ ID NO: 60 - protein, 260 aa, Escherichia coli
MLNRKLNIRLRHSLNSHCIPSIIINNTVRSFQRSVMNTRALFPLLFTVASFSASAGNWAVKNGWCQTMTEDGQALVMLKNGTIGITGLMQGCPNGVQTLLGSRISINGNLIPTSQMCNQQTGFRAVEVEIGQAPEMVKKAVHSIAERDVSVLQAFGVRMEFTRGDMLKVCPKFVTSLAGFSPKQTTTINKDSVLQAARQAYAREYDEETTETADFGSYEVKGNKVEFEVFNPEDRAYDKVTVTVGADGNATGASVEFIGK

SEQ ID NO: 61 - protein, 385 aa, Escherichia coli
VVIINSTILSGAGAIPSLTSLLPDIRKMLLVTDRNIAQLDGVQQIRALLEKHCPQVNVIDNVPAEPTHHDVRQLMDAPGDASFDVVVGIGGGSVLDVAKLLSVLCHPQSPGLDALLAGEKPTQRVQSWLIPTTAGTGSEATPNAILAIPEQSTKVGIISQVLLPDYVALFPELTTSMPAHIAASTGIDALCHLLECFTATVANPVSDNAALTGLSKLFRHIQPAVNDPQDLRAKLEMLWASYYGGVAITHAGTHLVHALSYPLGGKYHLPHGVANAILLAPCMAFVRPWAVEKFARVWDCIPDAETALSAEEKSHALVTWLQALVNQLKLPNNLAALGVPPEDIASLSEAALNVKRLMNNVPCQIDLQDVQAIYQTLFPQHPFKE

SEQ ID NO: 62 - protein, 105 aa, Escherichia coli
MNIRKLFCPGNTPRILLFLFFFVVSAITTIACGYTEKNATGNVLLLFLLLLLAHRNTLTSITALLFLFCCALYAPAGMTYGKINNSFIVALLQTTTDEAAEFTGM

SEQ ID NO: 63 - protein, 147 aa, Escherichia coli
MNIQAIKEMVNLICSFLFIFFLSSAFVSFGCYAIYELFLWNDIIVYSWGYILIVFLPFTLYVMSFEILFFAISGRRLSKVTMVRLWLIIKIIIAFSICAVLIFSSIYKKELLSRNYIACSGIPSGWMPGLATKYVKEKSLCEKNGNN

SEQ ID NO: 64 - protein, 178 aa, Escherichia coli; identical to SEQ ID NO: 37

SEQ ID NO: 65 - protein, 209 aa, Escherichia coli
MFLKRKWYYAVTTSVVITLCGGGYYMYRQEYQMVVTVPTADANDPNWPNKRIQFDTSEWLQQLQYIKIDDHYILNTQYTPIANLDDFGITLKLQNALNGSDKRLPALYGLAEMDAQKFKDLMRGKIKCEYLRTTFDAETLKPVNDYFLISFTYKDKWYEFETERKISKTSDDGYFLWAFDNTVHEAGYWHNTDPAAYSYRDYQNGKAVK

SEQ ID NO: 66 - protein, 424 aa, Escherichia coli
MDIWRGHSFLMTISARFRQYVFSLMSILLQERKMNIFTLSKAPLYLLISLFLPTMAMAIDPPERELSRFALKTNYLQSPDEGVYELAFDNASKKVFAAVTDRVNREANKGYLYSFNSDSLKVENKYTMPYRAFSLAINQDKHQLYIGHTQSASLRISMFDTPTGKLVRTSDRLSFKAANAADSRFEHFRHMVYSQDSDTLFVSYSNMLKTAEGMKPLHKLLMLDGTTLALKGEVKDAYKGTAYGLTMDEKTQKIYVGGRDYINEIDAKNQTLLRTIPLKDPRPQITSVQNLAVDSASDRAFVVVFDHDDRSGTKDGLYIFDLRDGKQLGYVHTGAGANAVKYNPKYNELYVTNFTSGTISVVDATKYSITREFNMPVYPNQMVLSDDMDTLYIGIKEGFNRDWDPDVFVEGAKERILSIDLKKS

SEQ ID NO: 67 - DNA, 489 bp, Escherichia coli
atgaaactgaaagctattatattggccaccggtcttattaactgtattgtattttcagcacaggcagtggatacgacgattactgtgacgggtaatgttttgcaaagaacatgtaatgtaccagggaatgtggatgtttctttgggtaatctgtatgtatcagactttcccaatgcaggaagtggatctccatgggttaattttgatctgtctctcaccggatgccagaatatgaatactgttcgggcaacatttagtggtactgcggatgggcagacatactatgcgaatacagggaatgctggcggtatcaagattgaaattcaggacagggatggaagtaatgcatcatatcacaatggtatgttcaagacgcttaatgtacaaaataataatgcaacctttaatcttaaagcccgtgcagtgagtaaaggccaggttactcctggaaatatcagttctgttataaccgtcacctatacctatgcg

SEQ ID NO: 68 - DNA, 2019 bp, Escherichia coli
atgaaaatgacacggctttatcctctggccttggggggattattgctccccgccattgctaatgcccagacttcacagcaagacgaaagcacgctggtggttaccgccagtaaacaatcttcccgctcggcatcagccaacaacgtctcgtctactgttgtcagcgcgccggaattaagcgacgccggcgtcaccgccagcgacaaactccccagagtcttgcccgggctcaatattgaaaatagcggcaacatgcttttttcgacgatctcgctacgcggcgtctcttcagcgcaggacttctataaccccgccgtcaccctgtatgtcgatggcgtccctcagctttccaccaacaccatccaggcgcttaccgatgtgcaaagcgtggagttgctgcgaggcccacagggaacgttatatggcaaaagcgctcagggcgggatcatcaacatcgtcacccagcagccggacagcacgccgcgcggctatattgaaggcggcgtcagtagccgcgacagttatcgaagtaagttcaacctgagcggccccattcaggatggcctgctgtacggcagcgtcaccctgttacgccaggttgatgacggcgacatgattaaccccgcgacgggaagcgatgacttaggcggcacccgcgccagcatagggaatgtgaaactgcgtctggcgccggacgatcagccctgggaaatgggctttgccgcctcacgcgaatgtacccgcgccacccaggacgcctatgtgggatggaatgatattaagggccgtaagctgtcgatcagcgatggttcaccagacccgtacatgcggcgctgcactgacagccagaccctgagtgggaaatacaccaccgatgactgggttttcaacctgatcagcgcctggcagcagcagcattattcgcgcaccttcccttccggttcgttaatcgtcaatatgtctcagcgctggaatcaggatgtgcaggagctgcgcgctgcaaccctgggcgatgcgcgtaccgttgatatggtgtttgggctgtaccggcagaacacccgcgagaagttaaattcagcctacgacatgccgacaatgccttatttaagcagtaccggctataccaccgctgaaacgctggccgcatacagtgacctgacctggcatttaaccgatcgttttgatatcggcggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcgggtcttgatgccaaaccgttcgtcgccgagaaatccatcaactatgaacttggcacccgctacgaaaccgctgacgtcacgctgcaagccgcgacgttttatacccacaccaaagacatgcagctttactctggcccggtcgggatgcagacattaagcaatgcgggtaaagccgacgccaccggcgttgagcttgaagcgaagtggcggtttgcgccaggctggtcatgggatatcaatggcaacgtgatccgttccgaattcaccaatgacagtgagttgtatcacggtaaccgggtgccgttcgtaccacgttatggcgcgggaagcagcgtgaacggcgtgattgatacgcgctatggcgcactgatgccccgactggcggttaatctggtcgggccgcattatttcgatggcgacaaccagttgcggcaaggcacctatgccaccctggacagcagcctgggctggcaggcgactgaacggatgaacatttccgtctatgtcgataacctgttcgaccgtcgttaccgtacctatggctacatgaacggcagcagcgccgtcgcgcaggtcaatatgggtcgcaccgtcggtatcaatacgcgaattgatttcttc

SEQ ID NO: 69 - DNA, 738 bp, Escherichia coli
atgaataaggtttttgttgtttcagtggtggccgcagcctgtgtatttgcagtaaatgcaggagcaaaggaaggtaaaagcggtttttatctgaccggtaaagccggtgcctctgtgatgtcactttcagaccagcgtttcctgtcaggagatgaggaagaaacatcaaagtataaaggcggcgatgaccatgatacggtattcagtggcggtattgcggtcggttatgatttttatccgcagttcagtattccggttcgtacagaactggagttttacgctcgtggaaaagctgattcgaagtataacgtagataaagacagctggtcaggtggttactggcgtgatgacctgaagaatgaggtgtcagtcaacacactaatgctgaatgcgtactatgacttccggaatgacagcgcattcacaccatgggtatccgcagggattggctacgccagaattcaccagaaaacaaccggtatcagtacctgggattatgagtacggaagcagtggtcgcgaatcgttgtcacgttcaggctctgctgacaacttcgcatggagccttggcgcgggtgtccgctatgacgtaaccccggatatcgctctggacctcagctatcgctatcttgatgcaggtgacagcagtgtgagttacaaggacgagtggggcgataaatataagtcagaagttgatgttaaaagtcatgacatcatgcttggtatgacttataacttc

SEQ ID NO: 70 - DNA, 498 bp, Escherichia coli
atgaaactgaaagctattatattggccaccggtcttattaactgtattgcattttcagcacaggcagtggatacgacgattactgttacagggagggtattgccacgtacctgtaccattggtaatggaggaaacccaaacgccaccgttgttttggataacgcttacacttctgacctgatagcagccaacagcacctctcagtggaaaaatttttcgttgacattgacgaattgtcagaatgtaaacaatgttacttcatttggtggaaccgcagaaaatacaaattattacagaaatacaggggatgctactaatatcatggttgagctacaggaacaaggtaatggtaatacccccttgaaagttggttcaacaaaagttgttacagtgagcaatgggcaggcgacattcaatcttaaagtccgtgccgtaagcaaaggtaatgctggtgcgggaagtattaattcacaaattactgtcacctatacctatgcg

SEQ ID NO: 71 - DNA, 3885 bp, Escherichia coli
atgaataaaatatactcccttaaatatagtgctgccactggcggactcattgctgtttctgaattagcgaaaagagtttctggtaaaacaaaccgaaaacttgtagcaacaatgttgtctctggctgttgccggtacagtaaatgcagcaaatattgatatatcaaatgtatgggcgagagactatcttgatcttgcacaaaataaaggtattttccagcccggagcaacagacgtaacaatcactttaaaaaacggagataaattctctttccataatctctcaattccggatttttctggtgcagcagcgagtggcgcagctaccgcaataggaggttcttatagtgttactgttgcacataacaaaaagaaccctcaggccgcagaaacccaggtttacgctcagtcttcttacagggttgttgacagaagaaattccaatgattttgagattcagaggttaaataaatttgttgtggaaacagtaggtgccaccccggcagagaccaaccctacaacatattctgatgcattagaacgctacggtatagtcacttctgacggttcaaaaaaaatcataggttttcgtgctggctctggaggaacatcatttattaatggtgaatccaaaatctcaacaaattcagcatatagccatgatctgttaagtgctagtctatttgaggtcacccaatgggactcatacggcatgatgatttataaaaatgataaaacatttcgtaatcttgaaatattcggagacagcggctctggagcatacttatatgataacaaactagaaaaatgggtattagtcggaacaacccatggtattgccagcgttaatggtgaccaactgacatggataacaaaatacaatgataaactggttagtgagttaaaagatacctatagtcataaaataaatctgaatggcaataatgtaaccattaaaaacacagatataacattacaccaaaacaatgcagataccactggtactcaagaaaaaataactaaagacaaagatattgtgttcacaaatgggggagatgtcctgtttaaggataatttggattttggtagcggtggtattatctttgacgaaggccatgaatataacataaacggtcagggatttacatttaaaggagcaggaattgatatcggaaaagaaagcattgtaaactggaatgcattgtattccagtgatgatgttttacacaaaataggccccggtactctgaatgttcaaaaaaaacagggggcaaatataaagataggtgaaggaaatgttattcttaatgaagaaggaacatttaacaatatataccttgcaagcggaaatggtaaggtaatactaaataaagataattcccttggcaatgatcaatatgcggggatattttttactaaacgtggtggtacgctagatttaaatggacacaatcagacttttactagaattgccgccactgacgatggaacaacaataactaactcagatacaacgaaagaagccgttctggcaatcaataacgaagactcctacatatatcatgggaacataaatggcaatataaaactaacgcacaatattaattctcaggataagaaaactaatgcaaaattaattctggatggtagtgtcaacacaaaaaatgatgttgaagtcagtaatgccagtcttaccatgcaaggccatgcaacagagcatgcaatattcagaagctcagcgaatcattgctccctggtatttctttgtggaacggactgggtcaccgttttgaaagaaacagagagttcatataataaaaaattcaattctgattacaaaagtaataatcagcagacctcatttgatcagcctgactggaaaaccggggtgtttaaatttgatacattacacctgaacaatgctgacttttcaatatcacgcaatgccaatgttgaaggaaatatatcagcaaataaatcagctatcacaatcggcgataaaaatgtttacattgataatcttgcagggaaaaatattactaataatggttttgacttcaaacaaactatcagtactaatctatccataggagaaactaaatttacaggtggcatcactgcacataacagccaaatagccataggtgatcaagctgtagttacacttaatggtgcaacctttctggataatactcctataagtatagataaaggagcaaaagttatagcacaaaattccatgttcacaacaaaaggtattgatatctccggtgaactgactatgatgggaatccctgaacagaatagtaaaactgtaacgccgggtctccactacgctgctgatggattcaggctgagtggtggaaatgcaaatttcattgccagaaatatggcatctgtcaccggaaatatttatgctgatgatgcagcaaccattactctgggacagcctgaaactgaaacaccgactatatcgtctgcttatcaggcatgggcagagactcttttgtatggctttgataccgcttatcgaggcgcaataacagcccccaaagctacagttagcatgaataatgcgatctggcatctaaatagccagtcatcaattaatcgtctagaaacaaaagacagtatggtgcgttttactggtgataatgggaagtttacaacccttacagtgaacaaccttactatagatgacagtgcatttgtgctgcgtgcaaatctggcccaagcagatcagcttgttgtcaataaatcgttgtctggtaaaaacaaccttctgttagtcgacttcattgagaaaaatggaaacagcaacggactgaatatcgatctggtcagcgcaccaaaaggaactgcagtagatgtctttaaagctacgactcggagtattggcttcagtgatgtaacaccggttatcgagcaaaagaacgatacagacaaagcaacatggactctgatcggctataaatctgtggccaacgccgatgcggctaaaaaggcaacattactgatgtcaggcggctataaagccttccttgctgaggtcaacaaccttaacaaacgtatgggtgatctgcgtgacattaacggtgagtccggtgcatgggcccgaatcattagcggaaccgggtctgccggcggtggattcagtgacaactacacccacgttcaggtcggtgcggataacaaacatgaactcgatggccttgacctcttcaccggggtgaccatgacctataccgacagccatgcaggcagtgatgccttcagtggtgaaacgaagtctgtgggtgccggtctctatgcctctgccatgtttgagtccggagcatatatcgacctcatcggtaagtacgttcaccatgacaacgagtataccgcaactttcgccggccttggcaccagagactacagctcccactcctggtatgccggtgcggaagtcggttaccgttaccatgtaactgactctgcatggattgagccgcaggcggaacttgtttacggtgctgtatccgggaaacagttctcctggaaggaccagggaatgaacctcaccatgaaggataaggactttaatccgctgattgggcgtaccggtgttgatgtgggtaaatccttctccggtaaggactggaaagtcacagcccgcgccggccttggctaccagtttgacctgtttgccaacggtgaaaccgtactgcgtgatgcgtccggtgagaaacgtatcaaaggtgaaaaagacggtcgtatgctcatgaatgttggtctcaacgccgaaattcgcgataatcttcgcttcggtcttgagtttgagaaatcggcatttggtaaatacaacgtggataacgcgatcaacgccaacttccgttactctttc

SEQ ID NO: 72 - DNA, 426 bp, Escherichia coli
atgattaatattcccagtcccaccgctgttgttatggcgctggtagccatcagcacgcttcccagccctagcagggtaaagcttatgccatatcctcccagagcccacaacaccacaggtttactgccagtacgggaaatttgctttccccaccacggggacgatggcagaaacagcattgagccaagcatcagcagggcagcccatacagacagactcagatttgtctgtatgaccagaacagggagcacaaccagcagaccgttctgcccgataccgagaagcccggcactgaacgcaagtggccagcaggacagtggtttttggggcgtatcttcgatcccaggtgacattttaatgtttcaactccatgtattaattgtgtttatttgtaaaattaatttatctgacaataacatttcttat

SEQ ID NO: 73 - DNA, 954 bp, Escherichia coli
atgtatgcccgcgagtatcgctcaacacgcccgcataaagcgattttctttcatctttcttgcctcacccttatctgtagtgcgcaagtttatgcgaagccggatatgcggccactggggccgaatatagccgataaaggctccgtgttttaccatttcagcgccacctctttcgactctgtcgatggcacacgccattatcgggtatggacggccgtgccgaatacaaccgcaccggcatcgggttacccgattttatatatgcttgacggtaacgcagttatggaccgcctggatgacgaactgctcaaacaattgtcagaaaaaacaccgccagtgatcgtggctgtcgggtatcagaccaacctccctttcgatctcaacagcagggcttacgactatacgccagcagcagaaagcagaaaaacagatctccactcagggcgttttagccgtaagagtggtggcagcaacaacttccgccagttactggaaacgcgtattgccccaaaagtggaacagggactgaatatcgatcggcaacgccgcggcttatgggggcactcctacggcggcctcttcgtgctggattcctggctgtcctcctcttacttccggtcgtactacagcgccagcccgtcgttgggcagaggttatgatgctttgctaagccgcgttacggcggttgagcctctgcaattctgcaccaaacacctggcgataatggaaggctcggcgacacagggtgataaccgggaaacgcatgctgtcggggtgctgtcgaaaattcataccaccctcactatactgaaagataaaggcgtcaatgccgtattttgggatttccccaacctgggacacgggccgatgttcaatgcctcctttcgccaggcactgttagatatcagtggtgaaaacgcaaattacacagcaggttgtcatgagttaagccac

SEQ ID NO: 74 - DNA, 2175 bp, Escherichia coli
atgagaattaacaaaatcctctggtcgctaactgtgctcctagttgggttgaatagccaggtatcagtagccaaatactccgacgatgataatgacgagactctggtggtggaagccaccgctgagcaggtattaaaacagcagccgggcgtgtcggttattaccagcgaggatattaaaaagacccctccggtaaacgacctttcagatattattcgtaaaatgcctggtgttaatcttaccggcaatagcgcctcgggcacacgcggtaataaccgccagatcgatattcgtggtatggggccggaaaacaccttaattttaattgatggtgtaccggtgacgtcacgtaactccgtgcgttatagctggcgtggggagcgtgatacccgcggtgacaccaactgggtgccaccggaacaggttgagcgtattgaagtgatccgcggccctgcggcggcgcgctacggttcgggggccgccgggggggtggtgaacatcattaccaaacgtcccaccaacgactggcacggttcgctgtcgttatacaccaaccagccggaaagtagcgaagagggcgctacgcgtcgcgccaatttcagccttagtgggcctctggctggtgatgctcttaccacgcgtttgtatggtaacctgaataaaacggatgctgacagttgggatattaattctccggtcggtacgaaaaacgcagccgggcatgaaggggtacgtaacaaagatattaacggcgttgtctcgtggaaattaaatccgcagcagattctcgatttcgaagtcggatatagccgccaggggaatatctatgcgggcgatacgcagaacagttcttccagtgcagttaccgaaagcctggcaaaatccggcaaagagacgaaccgcctgtaccgacagaattatggcattacgcataatggtatctgggactggggacaaagtcgctttggtgtttattacgagaaaaccaataatacccgcatgaatgaaggattatccggcggtggtgaaggacgtattttagcgggtgaaaagtttacgaccaatcgcctgagttcctggcgaaccagcggtgagcttaatattcctttgaatgtgatggttgatcaaacgctgaccgttggtgcagagtggaaccgcgataagctcgatgatccttcctctaccagcctgacggtgaatgacagagatatcagcggtatttctggctctgctgcggatcgcagcagtaaaaatcattctcaaatcagtgcgctgtatattgaagataacattgagccggttcctggcacgaatatcattcccggcctgcgctttgattatctcagcgactccggcgggaacttcagccccagtctgaatctttcgcaggaattgggcgattatttcaaagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattggactggagttcacctgggaagattaccacgcaagtgtgacctacttccgcaatgattaccagaataagatcgtggccggggataacgttatcgggcaaaccgcttcaggcgcatatatcctcaagtggcagaatggcgggaaagctctggtggacggtatcgaagccagtatgtctttcccactggtgaaagagcgtctgaactggaataccaatgccacatggatgatcacttcggagcaaaaagacaccggtaatcctctgtcggtcatcccgaaatatactatcaataactcgcttaactggaccatcacccaggcgttttctgccagcttcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtcagtaatatcctcaataaacagatcttccgatcttctgaaggggcgaatacctataacgagccaggccgggcttattatgccggagttaccgcatcattc

SEQ ID NO: 75 - DNA, 3042 bp, Escherichia coli
atgggtaaccaatggcaacaaaaatatcttcttgagtacaatgagttggtatcaaatttcccttcacctgaaagagttgtcagcgattacattaagaattgttttaaaactgacttgccgtggtttagtcggattgatcctgataatgcttatttcatctgcttttctcaaaaccggagtaatagcagatcttatactggatgggatcatcttgggaaatataaaacagaagtactgacactcactcaagccgctcttattaatattggttatcgttttgatgtttttgatgatgcaaattcaagcacaggaatttataaaacaaagagtgcagatgtgtttaacgaagaaaatgaagaaaaaatgctcccgtcggaatacctgcattttttacaaaagtgtgattttgcaggtgtttatggaaaaactctgtcagattactggtcgaaatactatgataaatttaagcttttactaaaaaattattatatttcttctgctttgtatctttataaaaatggagagcttgatgagcgtgaatataatttctccatgaacgccttaaatcgcagtgataatatatcactattattctttgatatttatggatattacgcatctgatatttttgtagccaaaaataatgataaggtaatgcttttcattcctggtgcaaaaaaaccttttttattcaagaagaatatcgctgatttgcggcttacccttaaagaacttattaaggatagtgacaacaaacaattactttcccaacatttttcattatatagtcgtcaagatggagtttcctatgcaggagtaaattctgttctacatgcaatagaaaatgatggtaattttaatgagtcttactttctgtattccaataagacacttagcaataaagatgtttttgatgctatagctatttctgttaagaaacgcagtttcagtgatggtgatatcgttataaaatcaaacagtgaagctcaacgagactatgctctgactatactccagacgattttatcaatgacccctatatttgatatcgtagtcccggaggtatctgttccgcttggactggggattattacttccagtatggggatcagttttgatcaactgattaatggtgatacttatgaagaacgtcgttctgctatacctggtttggcgacaaatgcagtattgcttggtctgtcttttgcaattccactcttgattagtaaggcaggaataaaccaggaggtacttagcagcgttataaataatgagggcaggactctgaatgaaacaaatatcgatatatttttgaaggaatatggaattgctgaagatagtatatcctcaactaatttgttagacgttaagcttaaaagttccgggcagcatgtcaatattgtaaagcttagtgatgaagataatcaaattgtcgctgtaaaagggagttctctgagcggcatctactatgaagtggacattgaaacaggatatgagattttatcccgaagaatttatcgtaccgaatataataatgaaattctctggactcgaggtggtggtctaaaaggggggcagccatttgattttgaaagtctcaatattcctgtattttttaaagatgaaccctattctgcagtgaccggatctccgttatcatttattaatgatgacagctcacttttatatcctgatacaaacccaaaattaccgcaaccaacgtcagaaatggatattgttaattatgttaagggttctggaagctttggggatagatttgtaactttgatgagaggagctactgaggaagaagcatggaatattgcctcttatcatacggctgggggaagtacagaagaattacacgaaattttgttaggtcagggcccacagtcaagcttaggttttactgaatatacctcaaatgttaacagtgcagatgcagcaagcagacgacactttctggtagttataaaagtgcacgtaaaatatatcaccaataataatgtttcatatgttaatcattgggcaattcctgatgaagccccggttgaagtactggctgtggttgacaggagatttaattttcctgagccatcaacgcctcctgatatatcaaccatacgtaaattgttatctctacgatattttaaagaaagtatcgaaagcacctccaaatctaactttcagaaattaagtcgcggtaatattgatgtgcttaaaggacggggaagtatttcatcgacacgtcagcgtgcaatctatccgtattttgaagccgctaatgctgatgagcaacaacctctctttttctacatcaaaaaagatcgctttgataaccatggctatgatcagtatttctatgataatacagtggggctaaatggtattccaacattgaacacctatactggggaaattccatcagactcatcttcactcggctcaacttattggaagaagtataatcttactaatgaaacaagcataattcgtgtgtcaaattctgctcgtggggcgaatggtattaaaatagcacttgaggaagtccaggagggtaaaccagtaatcattacaagcggaaatctaagtggttgtacgacaattgttgcccgaaaagaaggatatatttataaggtacatactggtacaacaaaatctttggctggatttaccagtactaccggggtgaaaaaagcagttgaagtacttgagctacttacaaaagaaccaatacctcgcgtggagggaataatgagcaatgatttcttagtcgattatctgtcggaaaattttgaagattcattaataacttactcatcatctgaaaaaaaaccagatagtcaaatcactattattcgtgataatgtttctgttttcccttacttccttgataatatacctgaacatggctttggtacatcggcgactgtactggtgagagtggacggcaatgttgtcgtaaggtctctgtctgagagttattctctgaatgcagatgcctccgaaatatcggtattgaaggtattttcaaaaaaattt

SEQ ID NO: 76 - DNA, 1362 bp, Escherichia coli
atggtggacatgattaatgaaagtgcacggcaaacgccagtcattgcacaaacggacgttctggttatcgggggcggtccggcaggattatccgctgccattgcggcagggcggttaggtgccagaaccatgattgttgagcgctacgggtcgctaggcggcgtattgacgcaggtcggggtagaaagttttgcctggtatcgtcatccggggacggaagattgtgaagggatctgtcgtgagtatgaaggccgcgcacgagcgctgggtttcacacgaccagaacctcagtcaattagcgaagttatagatactgaaggatttaaagttgtcgccgatcagatgattacggaatctggcgttgagccgttatatcactcctgggttgtggacgtgatcaaggacggggatacgttatgcggtgttatcgtcgagaataaatcaggtcgaggggcaattctggcgaaaagaatcgtcgattgcacgggggatgctgatattgccgctcgtgcaggcgcgccctggacgaaacggagcaaggaccaactgatgggcgtcaccgtgatgttcagttgcgcaggtgttgatgtggcacgctttaaccgttttgttgcggaagaacttaagccgacctacgcggattggggcaaaaactggacgattcaaaccacgggtaaagaagacccgatgtttagcccgtatatggaggatatttttacccgcgcgcaacaggatggtgtgattccaggtgacgcccaggcgattgccggaacctggtcgaccttttctgaaagcggtgaggctttccagatgaatatggtgtacgcctttggttttgactgtaccgatgtcttcgatttaaccaaagctgagattgccggaaggcagcaagcattatgggcaattgacgcactacgccactatgttccgggctttgaaaatgtacggttacgcaattttggtgccacgctggggacgcgtgaatcacggcttattgagggggaaatacgtattgctgatgattacgtccttaatcaggggcgttgttcggacagtgtagggattttcccggaatttattgatggttccggttatctcattttgccaacgaccgggcgtttctttcagatcccttatggttgtctggtgccgcaaaaagtggagaaccttttggtcgccggtcgctgtatttccgcaggcgtagttgcacatacttctatgcgtaacatgatgtgttgtgccgttaccggtgaggccgcaggtactgccgccgtggtttcgctacagcaaaattgcaccgtgcgtcaggttgctatccctgatttgcaaaacacgctgcaacagcagggcgttcgtctggca

SEQ ID NO: 77 - DNA, 759 bp, Escherichia coli
atgtctgccaaaagacgacttcttattgcgtgtaccttgataacagctatctatcattttcctgcatattcttcattagaatataaaggaacctttggttcaataaatgcgggttatgcagactggaacagtggatttgtaaacactcaccgtggtgaagtatggaaagtgactgcggattttggggtaaattttaaagaagcagaattttactcattttatgaaagtaatgtactcaatcatgctgtagcagggagaaatcatacggtttcagcaatgacgcatgtcagactctttgactctgatatgacattctttggcaaaatttatggccaatgggataactcatggggtgacgatctggacatgttttatggattcggttacctcggctggaacggcgagtggggcttttttaaaccgtatattggattgcataatcaatctggtgactacgtatcagctaaatatggtcaaacgaatggttggaatggttatgttgttggctggacagcagtattaccatttacgttatttgacgaaaaatttgttttatctaactggaatgaaatagaactggacaggaacgatgcttacacggagcagcaatttggccggaacgggttaaatggcggtttaactattgcctggaagttctatcctcgctggaaagcaagtgtgacgtggcgttatttcgataataagctgggctacgatggctttggcgatcaaatgatttatatgcttggttatgatttc

SEQ ID NO: 78 - DNA, 1476 bp, Escherichia coli
atggccagtttgatcggccttgcagtttgcacagggaatgcttttagtcctgccttagccgcagaggctaaacaacctaatttagtcattattatggcggatgatttaggttatggcgatttagcaacatatggtcatcagatcgttaaaacacctaatatcgacaggcttgcccaggaaggggtcaaatttactgactactatgcccccgctcctttaagttcaccttcacgcgcagggctattaaccggccggatgccatttcgtactggaattcgctcatggattccttcaggcaaagatgttgccttagggcgtaacgaactcacgattgctaatctactcaaagcgcaagggtacgacacggcaatgatgggtaagctgcatctgaatgcaggcggcgatcgcaccgatcagccacaagcacaagatatgggctttgattactcactggctaatacggcgggctttgttaccgacgccacgctggataacgctaaagaacgcccgcgttatggcatggtttacccgacaggctggctacgtaatgggcaacccactccacgagccgataaaatgagcggtgagtatgtcagttcggaagtcgtcaactggctggataacaaaaaggacagcaagcctttcttcctctatgttgcttttaccgaagtgcatagccccctggcttcgcccaaaaaatacctcgacatgtactcacaatatatgagcgcgtatcagaagcagcatcctgatttattttatggcgactgggcagacaaaccctggcgtggtgtgggggaatattatgccaatatcagctatctggatgcacaggttggaaaagtgctggataaaatcaaagcgatgggtgaagaagataacacaatcgttatttttaccagtgataacggtccggtaacgcgtgaagcgcgcaaagtgtatgagctgaatttggcaggggaaacggatggattacgcggtcgcaaggataacctttgggaaggcggaattcgtgttccagccattattaaatatggtaaacatctaccacagggaatggtttcagatacacccgtttatggtctggactggatgcctactttagcgaaaatgatgaacttcaaattacctacagaccgtactttcgatggtgaatcgctggttcctgttcttgagcaaaaagcattgaaacgcgaaaagccattaattttcgggattgatatgccattccaggatgatccaaccgatgaatgggcgatccgtgatggtgactggaagatgattatcgatcgcaataataaaccgaaatatctctacaatctgaaatctgatcgttatgaaacacttaatctgatcggtaaaaaaccagatattgaaaaacagatgtatggtaagtttttaaaatataaaactgatattgataatgattctctaatgaaagccagaggtgataaaccagaagcggtgacctggggc

SEQ ID NO: 79 - DNA, 954 bp, Escherichia coli
gtgacaacaactatctgcgctatgggcgaattgctggccgagtttttgtcccgcaacccacatcaaaaattcactcagcctggggagtttatcgggccatttcccagcggtgcgccagcaatttttgctgctcaggtggcaaaactgtcccatcgggccatcttctttggatgtgttggtaatgatgattttgcccgactcattatagagcgtctccgtcatgaaggtgtcattaccgatgggatccatgttatgaacaatgccgtcacaggtacggcgttcgtgagttatcaaaatccccagcagcgggatttcgtctttaatatccctaacagcgcctgcggtttgtttactgccgagcacattgataaggatctgcttaaacagtgtaaccatctgcatattgtgggctcatcgttgttctcatttcgcatgatcgatgtcatgcgtaaagcaataacgacgatcaaatcggctggcggcaccgtttctttcgatcccaatattcgcaaagagatgctgagcattcctgaaatggcgcaggctctcgattatttgattgaatatacggatatttttatccccagcgaaagcgaactccctttcttcgcgcgtcacaaaaatctgtcagaggaacagattgttagcgatcttctccacggcggcgtaaaacatgtggcgataaaacgcgcccagcgtggggccagctattacaagcttaaaaacggtacattacacgcccagcatgttgcaggtcacgatatcgaaattatcgatccaacgggtgcaggcgactgctttggcgcaacgtttatcactcttttcttatccggtttcccggcacacaaggcgctgcaatatgcaaatgccagcggcgcgctcgccgtaatgcggcaaggtccgatggaagggatatcctcactggcagacattgaagactttttgcagcagcac

SEQ ID NO: 80 - DNA, 513 bp, Escherichia coli
atgaagatattcattagtttatttttgtttataatatcaacaaattcttttgctgatgatatcactcatgccggagtggttcgtattgaagggttaattaccgaaaaaacctgcattatttctgatgagtcaaaaaattttacagttaatatgccagacgtacccagtagttcggtaaggagtgcaggggatgttactgaaaaggtttatttttccataacgttaacccgctgtggtagtgatgttggcaacgcgtatataaagtttaccggcaatacagtttctgaagatgccagtttatataagctggaagatggctcggtagaggggcttgcacttacgatttttgataagaacaaaggcagtattagtaatgatgttaaaagcatggttttttcacttacatcatcagttgataatatattgcatttttttgcggcttacaaagcattaaaaaataatgtccaaccaggggatgcaaatgcgtcagtatcgtttattgtcacctatgat

SEQ ID NO: 81 - DNA, 603 bp, Escherichia coli
atgattaaattccggctttatattccccctgtaattctcggttttgttatcgtaccattattggtatggccgacggttattgccttagccgtacttatattcacgttaacttttctggcggaaataatattctcctttccgctcctggttgtgcgtatttctcttcaggaattacaacttgagttattggttgtatatgcactttttttcagtgtaatgggtggcatcggttggcaattctcccgcagaacgcctcctgaattaaaaaacaggctacattgctggctggtcttttctccggtctatttctggttaattctctcgaatttcattctttatatttctccagagaaatcagcgttgctggaaaatatccgaaatttctttctgacatttgtctggcttcccctgaatttttcccctttttggccgcagccgtggactgattttgtcggcccgattagtgcccagcttggttttgcgttgggatattattgccagtggcgtagcaaaaatagaagccataggaagaagtggggcgattgggtaacgtgcttaagtttggcgattttagctctggggccgttattcaattatttacaa

SEQ ID NO: 82 - DNA, 702 bp, Escherichia coli
atgaaattcaatttatctaatttatccgcagtattactggcatcaggtatgctgatgtctactgcggtaaccgcagcacccggcgatgcaacacaatttggtggggcggatactgactggagcaccgttgattatcccaggctcactgatatggatgacaacgttgattcaatgggggggaaaatccgctttactggccgtgtagtgaaagctacctgtaaggtcgcaaccgattcaaaacagattgaagttgtcctgccggttgtgccttccaaccttttcactggtatcgacgtagaagcacagggggcgagcaaccagaccgatttcaatattaatctgaccgaatgtagcaatacagatgatcagaaaattgagttccgttttaccggtactgcagatagcgctaataaaacgctcgctaacgaagtagaaggatcaacggatgctgacaacagcggcaatgcgggggcgactggtgtagggattcgaatttactccaaaggtacgacgaataatggtctgattaacctgaataccactgcggcagagggtagcgcctccaccgccgcttatacaattccaggaaatgctacgacccatgatttcagcgcggcctttactgcaggttatgctcaaaacggtagcactgttgcaccaggtgtagttaagtcaacagcaagttttgttgtgctgtacgag

SEQ ID NO: 83 - DNA, 1008 bp, Escherichia coli
atgcgtatacatacttattggtatagaagatatttcattttattgattattatattttcaaatgttctttcttctattgctaatgctgaagatatggggcgagaacgtgcatattgttatccgggttcaccgagtaataatactacgcctgcatccttttcttataattttggtactatagtggtttctgatgtcaacaaaaatgcgcctggcactgtattgccatcacaaatctggaaggttggaacctataaggcttattgtaattctcttgatgattatgaaatttacttcagtgctgtctctggaatagatccgtctggtgccagtggtgatcatcaagggagtgatgtatttattccactcacccatgaaatatctgtctctactcatataaaactttataatcaaaatggcacaatgacagataaaattgtgccattcgaaaattataataccaattatccgggggacagaagcaaaccatctaattgggcatcaggtactgaaggatatattaaaatcaggattgataaaaaaattatatctgatgtttcattaagtaacgtattattggtgtcattatatgtcagccagatccctaccgaacatggtcctatccctgtctttaatgcctacataggaaacttaaatattcaggttccgcaaggttgcactattaatgagggtacgagttttactgttaatatgccggatgtgtgggccagtgaattgagccgggctggtgccggagcgaagcccgctggtgttactcctgtagcaacaactattccgattaattgtacgaataaagatacagatgcggtaatgacgttggtattcgacggtaacatttccgccacacgtgataccaatgggaaacaaagtattattcaggcacaagataatcctgatgttggtattatgattatggatagtcagcaaaactccgtagatttaaatgccctggcaacatcagtaggcgttccgttcagattggtggaaaac

SEQ ID NO: 84 - DNA, 2592 bp, Escherichia coli
atgaacctaaagctcaaaagatgcgaatattggatggcggcacaaaagcagatgaaacgggttgtgccgcttcttctggttattatgcctgcatgttcaatcgcgggaatgcgctttaaccctgcttttctgtcgggtgatactgaagctgttgctgacttatcccgcttcgagaaagggatgacttatcttcctggtagctatgaagtcgaagtttgggtcaatgattcccctttactctctcgtactgtaacttttaaagcagacgatgagaatcaactgattccctgcctttcacttgctgacttattaagccttggaattaacaaaaatgcgctgccagagcaggctttggcttcatctgaaaatagttgccttgatttgcgtatctggtttcccgatgtgcattacatgccggagctggatgcacagagacttaaactgacctttccacaggcgataataaaacgtgacgctcgcggatatattccaccagaacagtgggataacggtattacagcttttttgctgaattatgacttttctggtaataacgatcgtggtgattactcttcaaataactattatttaaatcttcgcgctgggatcaatattggtgcatggcgttttcgcgattattcaacctggagtcgtgggagtaattcagcaggtaaactggagcatatcagtagtacgttgcagcgcgttattattcctttcagaagtgaattaacgctaggagatacatggtcatcatcagatgttttcgacagtgttagtattcgtggcataaaactggaatctgacgaaaatatgttgcccgatagtcaaagtggtttcgctcccacggtgcgcggaattgcgaaaagtcgcgctcaggtaacaatcaaacagaatggttatgtcatttatcaaacctatatgccgccgggaccgtttgagattagcgatcttaacccgacatcatctgcgggagatctggaagttaccatcaaagagtctgataattcagaaactgtctataccgtaccttatgccgctgtccccatcctgcaacgagaaggtcatttaaaatattctactacggttggccaatatcgaagcaatagctataaccagaaaagtccttatgtatttcagggggaattaatttggggtttaccctgggatattacggcttatggtggggcacaattctctgaggattaccgggcgttggcgctcggccttggcctgaatctgggtgtatttggtgcaacatcgtttgatgttactcaggctaacagttcgcttgtggatgggagcaaacatcaagggcaatcttatcgttttctttattccaaatcgttagttcagacaggaacagcattccatattattggctatcgttattcaacccagggcttttacactttaagtgatacgacataccaacaaatgtcagggactgttgttgatccaaaaacgttagatgataaagattacgtttataactggaatgatttttataacttgcgttatagcaaacgtggaaaatttcaggctagtgtatcgcaacctttcggtaactacgggtctatgtatttatcggctagtcagcaaacatactggaatactgataaaaaagattctttataccaagttggttataacaccagtattaagggtatctatctaaatgttgcgtggaattacagtaaatcaccagggacaaatgcggataaaattgtctcgctaaatgtctcattacctataagtaattggttatcttccacgaatgatgggcgctcatcatcgaatgccatgactgcaacgtatggttatagtcaggataaccacggacaggtaaaccaatatacgggggtatctggttctctgttggagcagcataatctcagttataacatacaacatggttttgctaatcaggataatagcagtagtggttctgttggtgttaattatcgtggggcatatggttccttgaattccgcctacagttacgataatgaaggtaatcaacaaataaactatggcatcagtggtgctcttgttgtacatgaaaatggtcttacgttgagtcaaccattaggtgaaactaatgttttgataaaagcgcctggagcgaataatgtggatgttcagcgggggacaggaatatccactgactggcgtggatatgcagttgttccttatgcaacagaatatagacgtaataatatttcattagatcctatgtcaatgaatatgcatactgaactggatatcacttccactgaagttattccgggaaaaggtgcgttagttcgtgcagagtttgctgctcatatcggtattcgtggtttgttcacagttcgttatcgtaataaatcagtcccattcggtgctacagccagcgctcagattaaaaacagtagtcaaattaccgggattgtcggcgataatggacaactttatctctcaggattgcctttagaaggtgttattaatatccagtggggagacggtgttcagcaaaaatgtcaggctaattacaagctccctgaaacagaactggataatcctgttagctatgcaactctggagtgccgc

SEQ ID NO: 85 - DNA, 507 bp, Escherichia coli
atgggagcgatttatgttaaacgtttgattctgtcggtagcactgataataccgatagcatccaatgcttctgatgctttgaaccagccgagcagtagtctaaatgatggtgttgagactttttttatttcctgctttgatatgcctcaggaaacaactactgatatggacgcttgtcagagagttcagttagctcaggttagttgggttaagaataagtattcggtggccgccctgaatcgtttgaaacaagacaacaaggatgatccacagcgtctgcaggaattaactgcttcttttaacgcggaaagtgaagcttggacagaattaattgagaaagcgtcaaagtccgtccaggttgattatgtaggaggaactatagctggcactgcagttgcatcacgtcaaattggtcttctggaattacaatcccacgatatctgggagcactggctacgatctcgaggactcaactcctcctcttttgccagaaccaaagttcaaatc

SEQ ID NO: 86 - DNA, 2139 bp, Escherichia coli
atggctatgttcacaccttcattctcaggactcaaaggtcgggcgctcttttcactgctttttgcggcaccgatgattcatgcaacagactctgtaacgaccaaagatggcgaaacaatcactgttacagcagatgcaaataccgcaactgaggcaaccgatggttatcaacctctgagcacctccacggcgacattaaccgatatgccgatgctggatatcccgcaggtggtcaatacggttagcgatcaggttctggaaaaccagaatgcgacaacgctggatgaggcgctttataacgtcagtaacgtggtacagaccaatacattaggcgggactcaggatgcttttgtacgccgtgggtttggcgcaaaccgggatggctccatcatgaccaacggtctgcgaaccgtacttcctcgtagtttcaacgccgcaacagagcgtgtggaagtgctaaaaggcccggcctccacgctgtatggcattctcgatcctggcggactgattaacgtcgtgaccaagcgcccggaaaaaacattccatggttcggtttcagccacctcctccagttttggtggcggcactgggcaacttgatatcacaggtcccattgaaggcactcagctggcgtatcgccttaccggggaagtgcaggatgaagattactggcgaaacttcggtaaagagcgcagtacatttattgccccgtcactcacctggtttggtgataatgcaacagtaaccatgctctattcccatcgggactataaaactccattcgatcgtggaacgattttcgaccttacgacgaaacagcccgtaaacgttgatcgaaaaatacgttttgacgaaccgtttaatattacagatggtcagtccgatctggcgcaactcaacgcagaatatcatctcaatagccagtggacagcgcgctttgattacagctacagccaggataaatacagcgataatcaggcgcgtgttaccgcgtatgatgcaacgacaggaacactgacacggcgtgttgatgcaactcagggatctacccagcgtatgcatgctactcgtgcggatctgcaagggaatgttgatattgccggattctataatgagattctgggtggggtgtcatatgaatattatgatcttctgcgtacagatatgattcgctgtaaaaaagctaaagatttcaatatatacaaccctgtttatggtaataccagcaaatgtacaacggtttcggcgtcggacagcgatcagacgatcaaacaggagaactactcagcttatgcacaggacgcgctctatctgaccgataactggattgccgtcgccgggatccgctatcagtattacacgcaatatgcgggtaaaggccgtccttttaatgtcaatactgacagccgcgatgaacaatggacgcccaaactggggttagtctacaaactgacgccatcggtatccttatttgccaattattcgcaaacatttatgccgcagtcgtcaattgccagctacattggcgatcttccaccagaatcatctaatgcttacgaagtcggggcaaaattcgagctattcgatggtatcaccgcagatattgcgctgtttgatatccataaacgtaatgtgttgtataccgaaagtattggtgatgaaaccatcgccaaaacggcaggccgcgttcgttcaagaggggtagaagtcgaccttgcgggagcattaactgaaaacattaatatcattgccagctacggctataccgatgcaaaggttctggaagatcctgattatgcagggaaaccattgccgaatgttcctcgtcataccggttcgctattcctgacctatgatattcataacatgccaggcaataacacactgacgtttggcggtggcggacatggtgtaagccgtcgttcggcaaccaatggggctgactattatctgcctggctatttcgttgccgatgccttcgccgcatacaaaatgaaattgcagtatccggtcactctgcaattaaacgtcaaaaacctgtttgataaaacgtattacacctcttccatcgccacaaataatctgggcaaccagattggcgatccgcgtgaagtgcaattcacggtgaaaatggaattt

SEQ ID NO: 87 - DNA, 1818 bp, Escherichia coli
atgaaaatatcgtggaattatatatttaagaacaaatggcgatttcacattacaagcatttcactttttcttatcatgctcgcggtttcaatcgcttttttgcacttgcgttttaataccttgtccagtaccgataaaatgcggcttgaaatgtataagtccacattatattccaccatcgagcaattttatgttttaccctatatgctctcaacagaccatatcatccgtcaggcggtaattacgcctgacgatatgacgtccagcgaactcaatcaacgaattgcacatttcaatactcaactcaaaaccgcagcaatatttattctggatacccaaggtaaggccatcgcttctagcaactggcaggaccccggcagctatgtagggcaaaattatagctatcgcccctattataaacacgccatgtctggcttaaatggacgcttttacggtattggtagcactacgaatacaccgggattcttcctctctacaagtataaaagataaaggaaaaattgtcggtgttgtagtagtaaaaataagtcttaatgaaattgaaaaagcatgggccgaaggtcctgaaaatattatcgtgaatgatgaacatgggattatatttttaagttcaaaatcgccatggcgaatgcgaacactgcaaccgttacctgttcaggcaaaacaaaaactacaatctacccgccaatatagtctcgacaatcttttaccggcggattattatccctgttataccgtgagcaattttactttcctgaaagataaaaaagaacaactctgtttattcccgcaatattatacgcaacaaatagccattccagaatttaactggaaaatgacaattatggtccccttagataacctgtactggtcatgggctatttcgttagtcattacactaattatttacctgctgtttttgttatttattaaatactggagaatgcgatctcatgcacaacaattattaacacttgcgaatgaaacattagaaaaacaggttaaagagcgtacatctgccctggaattgatcaatcaaaaattaatacaggagataaaagagcgcagtcaagctgaacaagtattacaaattacgcgtagtgaactggcagagtccagcaaactggcggcgcttggacagatggcaaccgaaattgcccatgaacaaaatcaaccgttagccgccattcacgcacttactgataacgcgcgtactatgctaaaaaaagagatgtatccgcaggttgaacagaatctgaaacatattatttcagtgattgagcggatgacgcagctcatttccgaacttaaagcatttgcctcgcgccatcgcgtacctaaaggttctgccgatgtcatcaaagtgatgtatagcgccgtggcgttacttaatcacagcatggagaaaaataacattgagcgacgaataaaagccccatccatgccgttatttgtcaattgcgatgagctcggtcttgaacagatattcagtaatttaattagcaacgccttagattctatggaaggtagctcttacaaacgactggatatcgccattcgccaggcaaataacaaagttattattaccattaaagacagcggtggcggttttgcacctgaagttgtcgatcgcatatttgaaccattttttaccactaaacgtagaggaatggggttgggactggcaatagtcagcgaaattgtccgaaattcgaacggcgcactccacgccagtaatcatcctgaaggcggcgcagtaatgacattaacctggcctgaatggggagaagaacatgaa

SEQ ID NO: 88 - DNA, 303 bp, Escherichia coli
gtgcttacaccacaacatttacgttgtgtgttaacatgtagcgatttactgactcttttgagtggtaccgttatgtctcaaatgcccctctattttcttaatacccaaaagaaactcactgctcactatgaatggcttcaaatcaacctgactgatacctacgaactagttaaaaggttaatgccgattccttcactggacgtggtggttaaagtagggaaacttgtcctcccggagaaagggcatcatggtttttaccctgaagctggagttgtctatagaacagtagctccagaaaatcca

SEQ ID NO: 89 - DNA, 789 bp, Escherichia coli
atgatgaaaaatacaggctatatcttagctctttgtctgacagcatcggggcatgtcctagcccatgatgtctggattacaggtaaacaggcagagaacaacgttaccgcagagattggttatggtcataatttcccctcaaaggggacaattcctgacagaagggatttctttgaaaatccccggctttataacgggaaagagacaataacactgaagccagcgtccacggattatgtctataaaactgagtctgcaagcaaagataatggttacgttctgtcaacgtatatgaaaccgggatactggtcgagaacctcgtcaggatggaaaccggtcagccgggagggcagaaatgatgtggcttactgtgaatttgtcactaaatatgcaaaatcttttattcctggtgaacagcagatgccagcacaactctatcagtctccaacagggcatgagcttgaaatcattccgttatccgatataagtcgtttcagtgaaaatgtgaagctgaaagttctgtataaaacgtccccgctcgccggagctatcatggagcttgactcggtcagttatctgacatcatcccgtcatactcatgcagttgagcacaaacatcctgttcataaagcagaactcacctttgtaactaatgaggatggtatcgtcacagtaccttctcttcatatcggacagtggctggcgaaagtccaaaataagaaaagttttcaggacaaaagcctgtgtgatgaaactgtcgatgtggcaaccttaagcttctcccgaaat

SEQ ID NO: 90 - DNA, 1134 bp, Escherichia coli
atgggaaaaataaaatattggctaatagtaggatttattatactttttgcgattttttacattgctattagtgacagggattctacgctttctaggttgaaatcagcaggtgaaaacggagatgtagaagctcagtatgctttggggctcatgtatttgtatggagaaattctggatgttgattatcagcaggcaaagatttggtatgaaaaagccgctgaccaaaatgatccgcgtgcgcaggccaaactcggtgtgatgtatgcaaatggtctcggggtaaatcaggattatcagcaatcaaaattatggtatgaaaaggcggctgcgcaaaatgatgttgatgcgcaatttttgcttggggagatgtatgacgatggtctcggggtaagccaagactaccagcatgcaaagatgtggtatgaaaaagcggctgctcaaaatgatgagcgtgctcaggtcaatctcgctgttctatacgcaaagggtaatggtgttgaacaggattatcgacaggccaaaagctggtatgaaaaggctgcagctcaaaatagtcctgatgcgcagttcgctcttggaattctgtatgccaatgctaatggtgtagagcaggactatcagcaggcaaaagactggtatgagaaagcagcagaacaaaatttcgccaatgctcagtttaatcttggtatgctctattacaaaggtgagggtgttaaacaaaactttcggcaagccagagaatggtttgaaaaagccgcatctcaaaatcagccgaatgcccaatataatttaggtcagatttattactacggtcagggtgtgactcagagctatcgacaggcgaaagactggtttgaaaaagcggcagagaaaggtcatgtcgatgctcaatataatctcggtgtaatatacgaaaatggtgaaggtgtgagtcagaactatcaacaggcaaaggcttggtatgaaaaggcagcctcacaaaatgatgcgcaggcgcagttcgaacttggcgttatgaatgaactgggtcagggtgaaagcatagacctgaaacaagcaagacattactatgagcggtcatgtaataatgggcttaagaaaggttgtgaacggttaaaagagttattatacaaa

SEQ ID NO: 91 - DNA, 1962 bp, Escherichia coli
atgaatgtaatcagaactgtcatttgtacattaattatacttccggtgggattacaggcagcgaccagtcattcttctatggttaaagatacaatcaccattgtcgcgacaggaaatcagaacacggtatttgaaacgccgtcgatggtcagtgtcgtcacgaatgacacaccgtggagtcagaatgcggttacatcggccggcatgctgaaaggtgttgccggtctcagccagactggtgcaggacggaccaatgggcagacctttaatttacgcggctatgacaaaagcggggtacttgttcttgttgacggcgttcgccaactcagtgacatggcaaaaagcagtggcacttatctggatccggcactcgtcaaacgtatcgaagttgtccgcgggccaaactccagtctgtacggcagtggcgggctgggaggtgtagtggacttcagaactgccgatgcagcagattttcttccccccggagagacaaacggtttaagtctgtggggaaatatcgccagtggtgaccacagcacaggctcggggctcacctggtttggtaaaactggaaaaacagatgcgctcctttctgtcattatgcgtaaaagaggtaatatctatcaaagtgatggtgagcacgcacctaacaaggaaaaacctgcagccctgtttgcgaaaggctctgtcggtataacagacagtaacaaagcaggtgccagcttgcgtctctaccggaataacaccactgaaccgggcaattccactcagacacatggtgacagcggcctgcgtgacagaaaaacagtacaaaatgacgtacagttctggtaccagtacgctcctgtggataacagcctcatcaatgtaaagtcaacgttatatctcagtgatatcactatcaagacaaacggtcacaacaaaacggcagaatggagaaacaacagaacctccggtgttaatgttgtcaacaggagtcatactctgatttttccgggagcccatcagttaagttatggcgctgaatattaccgtcagcagcagaagccagaaggctctgccacactatatccggaaggaaacattgactttacatcgttgtatttccaggatgaaatgacaatgaaaagctacccggttaacattatcgtcggttcccgctatgaccggtacaagagcttcaatccccgtgccggagaactgaaagccgaacgcctgtccccaagggcggcgatttcagtctcaccgacagactggctgatgatgtacggctccatatcctctgcattccgagcgcccacaatggcagaaatgtacagggatgatgtacatttttaccgcaagggtaaacccaattactgggttcctaaccttaatctgaaaccagaaaataacatcacccgtgagattggcgcaggtattcaactggatggcctgcttacagacaatgaccggctgcagttaaaaggcggatatttcggaacggatgccagaaactatattgccacacgcgtggatatgaaacggatgcgttcttattcttataatgtatcccgggcccgtatctggggatgggatatgcagggtaattaccagtctgattatgttgactggatgctttcttataaccggacggaaagtatggatgccagcagcagggaatggctgggctccggcaatcctgacacacttatcagtgacatcagcatacctgttggtcatagaggcgtttatgccggatggcgtgctgaactttcagcatcagccacgcatgtgaaaaaaggcgatccccatcaggctggttataccatacattccttttcactgtcttataagcctgtaagtgttaaaggctttgaggcgtcagtaactctggataatgccttcaacaagcttgccatgaatggcaaaggtgtgccgctttcaggcagaactgtcagtctttatacccgttatcagtgg

SEQ ID NO: 92 - DNA, 4128 bp, Escherichia coli
atgaataaaatatacgctctaaaatattgttatattactaacacagtaaaggttgtctctgaactagcccgaagggtatgtaaagggagtacccgcagaggaaaaagactttcagtacttacctctctggcactatctgcattactcccaaccgttgctggtgcatcaacggttggtggcaacaatccttaccagacataccgcgactttgcagaaaacaaagggcagtttcaggctggcgcaacaaacattcctatttttaataataaaggggaattagtaggacatcttgataaagcgcccatggttgattttagcagtgtgaatgtaagctcaaatcccggcgttgcaacattaattaacccgcaatatatagccagtgtaaaacataataaaggatatcagagcgtcagcttcggtgatggtcagaacagttaccatattgtggatcgtaatgaacacagttcatctgatctccacacaccaagacttgataagctcgtaactgaggttgctccggctaccgtaaccagctcatcaacagctgatatattgaacccttcaaaatactcggcattctacagggctggttcgggaagtcagtatattcaggatagtcagggtaagcgacattgggtaacaggtgggtatggttatctgacaggaggaatactcccgacatcattcttttatcacggctcagacggcattcagctgtatatggggggcaacatacatgatcatagcatcctgccctcttttggagaggccggcgacagtggttctccattatttggctggaatacggccaaagggcagtgggaactggtcggtgtttactcgggagtaggaggggggaccaatttgatatattctcttattcctcagagttttctctcacagatctattcagaggataatgacgctcccgtcttttttaatgcctcatccggcgcccccctgcaatggaaatttgacagcagcaccggcactggctctctgaaacagggttccgatgaatatgccatgcacgggcaaaaaggttctgacctgaacgcaggtaaaaatctgacattcctgggacataatggtcagattgacctggaaaactctgtcacgcagggtgccggttcactgacatttactgatgactacactgtcaccacttcaaacggaagtacctggaccggggccggtattattgtggacaaggatgcctccgtaaactggcaggttaatggtgtgaaaggtgacaacctgcataaaatcggcgaaggaaccctggttgtacagggaaccggtgttaatgagggcggcctgaaagtcggggatgggaccgttgtcctcaatcagcaggctgacagttcaggacacgttcaggcattcagtagcgtgaatattgccagcggccgcccgacagtcgtgctggcagacaaccagcaggttaatccggacaatatatcctggggctaccgggggggggttctggatgttaacgggaatgacctgacatttcataagctgaatgccgccgattatggcgcaactctcggtaacagcagtgataaaacggctaatatcactctggattatcagacgcgtccggcagacgtaaaagttaatgaatggtcatcatcaaacaggggaacagtaggttcattatatatttataataatccctatactcataccgtcgattattttatcctgaaaacaagtagttatggctggttccctaccggtcaggtcagtaacgagcactgggaatatgtcggacatgaccagaacagtgcacaggcactgcttgcaaacagaattaataataaagggtatctgtatcatggcaagttgctgggaaatattaatttctcaaataaagcaaccccgggtacaaccggcgcattggttatggacggctcagcgaatatgtccggtacatttactcaggaaaacggtcgtctgaccattcagggccacccggttatccatgcttcaacgtctcagagtattgcaaatacagtctcgtctctgggcgacaattccgttctgacacagcccacctcatttacacaggatgactgggagaacaggacgttcagctttggttcgctcgtgttaaaagatacagactttggtctgggccgcaatgccacactgaacacaaccatccaggcagataactccagcgtcacgctgggcgacagtcgggtatttatcgacaaaaaagatggccagggaacagcatttacccttgaagaaggcacatctgttgcaactaaagatgcagataaaagcgtcttcaacggcaccgtcaacctggataatcagtcagtgctgaatatcaatgagatattcaatggcggaatacaggcgaacaacagtaccgtgaatatctcctcagacagtgccgttctggagaactcaacgctgaccagtaccgccctgaatctgaacaagggagcaaatgttctggccagtcagagttttgtttctgacggtccggtgaatatttctgatgccaccctgagtctgaacagccgtcctgatgaggtatctcacacacttttacctgtatacgattatgccggttcatggaacctgaagggagacgatgcccgcctgaacgtggggccgtacagtatgttgtcaggtaatatcaatgttcaggataaagggactgtcaccctcggaggggaaggggaactgagtcctgacctgactcttcagaatcagatgttgtacagcctgtttaacgggtaccgcaatacctggagcgggagcctgaatgcaccggatgccaccgtcagcatgacagacacccagtggtcgatgaacggaaactccacggcaggaaatatgaaacttaaccggacaatagtcggttttaacgggggaacatcatcgttcacgacactgacaacagataatctggacgcggttcagtcagcatttgtcatgcgtacagaccttaacaaggcagacaaactggtgataaacaagtcggcaacaggtcatgacaacagcatctgggttaacttcctgaaaaaaccctctgacaaggacacgcttgatattccactggtcagcgcacctgaagcgacagctgataatctgttcagggcatcaacacgggttgtgggattcagtgatgtcacccccacccttagtgtcagaaaagaggacgggaaaaaagagtgggtcctcgatggttaccaggttgcacgtaacgacggccagggtaaggctgccgccacattcatgcacatcagctataacaacttcatcactgaagttaacaacctgaacaaacgcatgggcgatttgagggatattaacggcgaagccggtacgtgggtgcgtctgctgaacggttccggctctgctgatggcggtttcactgaccactataccctgctgcagatgggggctgaccgtaagcacgaactgggaagtatggacctgtttaccggcgtgatggccacctacactgacacagatgcgtcagcaggcctgtacagcggtaaaacaaaatcatggggtggtggtttctatgccagtggtctgttccggtccggcgcttactttgatttgattgccaaatatattcacaatgaaaacaaatatgacctgaactttgccggagctggtaaacagaacttccgcagccattcactgtatgcaggtgcagaagtcggataccgttatcatctgacagatacgacgtttgttgaacctcaggcggaactggtctggggaagactgcagggccaaacatttaactggaacgacagtggaatggatgtctcaatgcgtcgtaacagcgttaatcctctggtaggcagaaccggcgttgtttccggtaaaaccttcagtggtaaggactggagtctgacagcccgtgccggcctgcattatgagttcgatctgacggacagtgctgacgttcacctgaaggatgcagcgggagaacatcagattaatggcagaaaagacggtcgtatgctttacggtgtggggttaaatgcccggtttggcgacaatacgcgtctggggctggaagttgaacgctctgcattcggtaaatacaacacagatgatgcgataaacgctaatattcgttattcattc

SEQ ID NO: 93 - DNA, 1047 bp, Escherichia coli
atgattacactttttcgactactggcgattctttgccttttttttaacgtttcagcttttgctgttgattgctatcaggatgggtacagaggaacaaccctcataaatggagatttaccaacgttcaaaattccagagaatgcgcaacctgggcaaaaaatttgggagagcggagatattaatatcacagtttattgtgacaatgcaccaggatggtcaagtaataacccatcagaaaatgtctatgcctggatcaaattgccccaaataaatagtgccgatatgttgaataatccgtatttaacatttggcgtgacttataatggtgtagattatgaagggacaaatgaaaaaattgatactcatgcgtgcctggataaatatgaacaatactataatgggtattatcatgaccctgtatgcaatggcagcactcttcaaaaaaatgtaacatttaacgcccattttcgcgtctatgtaaaattcaaaagccgcccggcaggagatcagacggtaaactttggcacagtcaacgtgctgcaattcgacggtgaaggcggggcgaacatggcccccaacgcgaaaaatttacgctatgcgattacggggttagataatatttcattccttgactgtagtgtcgacgtccgcatttccccggaaagtcagatagtcaattttgggcagatcgctgcgaattccattgcaactttcccaccgaaggcagcattcagcgtttctaccataaaagacattgcgtctgattgtaccgaacagtttgatgttgcaaccagtttctttacttcagatacattatatgacaatacgcatctggaaataggtaacggcttgctcatgcgaattactgatcaaaaaacgcaagaagatattaaatttaaccagttcaaattatttagtacttatattcccggtcagagtgcggcaatggcaacccgcgattaccaggccgaattaacccaaaaacctggtgaaccactcgtctatggcccatttcagaaagacctgatagttaaaatcaactaccac

SEQ ID NO: 94 - DNA, 2520 bp, Escherichia coli
atgaacaataaaaacacgttttcccgggataagttatcccatgcaattaaaaatgccctgtctggcgttgtgtgttccctactcttcgttttgccagtccacgccgtagaattcaacgtcgatatgattgacgcagaagaccgtgagaatatcgacatctctcgttttgagaaaaaaggctatatcccccctggtagatacctcgttcgtgtgcaaataaataaaaatatgttgccacaaacgttaatactggaatgggtaaaagccgataatgaaagtggttcgttactctgcttaaccaaagaaaatttgactaatttcggtcttaatacggaatttattgaatcattgcaaaacatagctggcagcgaatgtctcgatttaagccaacgtcaggagttaacgacacgacttgataaagctacgatgatattatcgctaagtgttccccaggcatggttaaaataccaggcaacaaactggacgccaccagagttttgggataccggtatcaccgggtttatccttgattacaacgtgtacgccagccagtatgccccacatcacggagacagcacccaaaacgtcagctcctatggtacgttaggctttaacctcggcgcatggcgcttacgtagcgattaccaatataatcagaattttgctgatggacgctcggtaaaccgcgacagcgaatttgcgcgaacttatctgtttcgccctatcccctcctggtcgtcaaaattcactatgggccagtacgacctgagctccaatctttacgataccttccactttactggcgcatcgctggaaagtgatgaaagcatgctgccgccagatttacagggttatgcgccacaaattaccggcatcgcgcagaccaacgcgaaagtaactgtggcacaaaatggtcgtgtactttatcaaaccactgtcgcgccaggcccttttactatttctgatttggggcaatcgtttcaggggcagctggatgtcacagtggaagaagaagatggccgcaccagcaccttccaggttggctccgcatccattccctatttaacccgtaaagggcaagtgcgctataaaacgtcactgggaaaaccgacatccgtcgggcataacgatatcaataatccctttttctggacggcggaagcctcctggggctggctgaacaatgtgtcgttgtatggtggtggcatgttcaccgctgatgattatcaggctatcactaccggtattggctttaaccttaaccaattcggttcgctttcttttgatgtcactggagcagacgcgtctttacagcaacaaaatagcggcaatctgcgtggttacagctatcgcttcaactatgcaaagcatttcgaatcgacaggcagtcagattaccttcgcgggttatcgcttctcagataaagattacgtgtcgatgagtgagtacctcagctcgcgtaatggcgatgagtcaatcgataatgaaaaagagagttatgtcatttccttgaaccagtactttgaaacgctggaattaaactcttatctcaacgttacacgcaatacttattgggacagcgccagcaataccaactactccgtatctgtaagcaaaaactttgatattggcgatttcaaaggtatatctgcatcgctggcagtaagtcgaatccgctgggatgacgacgaagagaatcaatattacttctctttctctctacctttacaacaaaaccgcaacatctcctacagtatgcagcgaacgggaagcagtaatacttcgcagatgatttcctggtacgattcatcagatcgcaacaatatctggaatatttcagcgtcggcaacggacgacaatatacgtgatggcgaaccaacactgcgcggcagctaccagcactattcgccgtggggacgcctgaacattaatggcagtgtacagccgaatcagtacaattctgttaccgcaggctggtacggttcacttaccgctacacgtcatggtgtcgcccttcacgattatagctatggcgataacgcccgcatgatggtcgataccgatggcatctccggcattgaaatcaactctaaccgtaccgttaccaacgggctgggcatcgccgtgataccttcgttatcgaactacaccacctccatgttgcgggtgaacaataacgatctgccagaaggtgtcgatgtcgaaaactcggttattcgtactacgctcacccagggtgccatcggctacgcaaaactgaatgccaccaccggataccaaatcgtcggcgttattcgtcaggaaaatggccgcttccctccactaggtgtgaatgtcacggataaagcgacaggtaaagatgtgggcctggtagcggaagatggcttcgtttatctcagcggtattcaggaaaacagtattctgcatttaacctggggtgataatacctgtgaagtcacgccgccaaaccaaagtaacattagtgaaagcgcgataattttaccttgtaaaacagtcaaa

SEQ ID NO: 95 - DNA, 507 bp, Escherichia coli
ttgatgaacacaaaacagtctgttgctcaactcgccgtaccgcaccgcaagcgcctttcatcaacgatggtggtggcgctgttactttgtgtggttgctggcgcggtgatgattaatgccgctgattttccagcaactgccattgaaacggatcccggtgcaagtgccttccctaccttctatgcctgtgccctgattgtgctcgctgtcttgctggtgatacgcgatcttttgcaggcaaaaccagcctcttgcgccaacgcacaggaaaaaccggcattcaggaaaacagcaacaggaattgcggcaaccgcgttttatattgtggcgatgagctactgcggttatctcattactactcctgttttcctcatcgtcattatgacgttgatgggctacaggcgatgggtactcacaccgggtattgcgctgctgttaacggcaatcctctggttgctgtttgtcgaagcgttacaggtgccattgcctgtcggcacatttttcgaa

SEQ ID NO: 96 - DNA, 933 bp, Escherichia coli
atggtacttcttgcaggcgctgccctcagcattgcgcctgtacaggcagcctcctacccaaccaaacagatcgagttagtcgttccctacgctgccggaggcggtacggatctggttgcccgtgcctttgctgatgccgccaaaaaccatttacccgtcagcatcggggttatcaataaacctggcggaggcggtgctatcggcctgagtgaaatcgccgctgcccgccctaacggttacaaaattggtttaggcacggttgaactgaccacccttcccagcctcggaatggtgcgttttaaaaccagcgactttaaacccattgcccgtctgaatgcggatccggctgctatcacagtccgtgccgatgcgccgtggaatagctatgaagaatttatggcttactccaaagcgaatcccggaaaagtacgcattggtaactcaggcaccggagctatctggcatctggcggcagctgcactggaagacaaaacgggcacaaagttttctcatgtcccgtatgacggcgcagcccctgccattacaggcctgttaggcgggcatattgaagcggtttccgtaagcccaggagaagttatcaaccatgtgaatggcggcaagctgaagacactggtagtgatggcggatgagcgaatgaaaaccatgcctgacgtcccgacgttaaaagagaaaggcgttgatctctccatcggcacctggcgcggcctgattgtgtcgcaaaaaacgccgcaggatgtggtggatgttctggcaaaggcagcaaaagagacggctgaagagcctgcattccaggatgcactgcaaaagttgaatctcaactatgcatggcttgacgctgccagcttccagacccaaatcagcgaacaggaaaagtactttgacgagttgctgactcgcctgggcctgaaaaaa

SEQ ID NO: 97 - DNA, 2166 bp, Escherichia coli
atgctgcgatggaaacgctgtattattctaacatttatctctggtgctgctttcgcggcgccagagataaatgttaagcaaaacgaatcgttacctgatttaggtagccaggcagcacaacaggatgaacaaaccaacaagggtaaatcgctgaaagagcgcggagccgattacgtcatcaactccgccacgcaagggtttgaaaacttgacccctgaggcgctggaatctcaggccagaagctatctgcaaagtcaaatcacctcaaccgcacaatcttatattgaagacacactctctccctacggtaaggtccgtttgaacctctccattggtcagggcggcgatctggatggcagttccatcgattattttgttccctggtacgataatcaaaccactgtttatttcagccaattttctgcgcaacgaaaagaagatcgtacgatcgggaatattggccttggggtaaggtataattttgataaatatctattgggtggaaatatattttatgattatgactttacccgtggacatcgccgtttaggtttaggcgccgaagcctggacggattatttaaaattctcaggcaactattatcacccactttctgactggaaagactctgaagatttcgacttttatgaagaacgccctgcgcgcggttgggatattcgtgccgaagtctggttaccttcttatccgcaactggggggcaaaattgtcttcgagcaatattacggcgatgaagtcgccctttttggtacggataatttggagaaagatccctacgcggtaacgcttggactgaattatcaaccagtgccgttactgacagttgggacggactataaagcggggaccggagataacagtgatgtcagcattaatgccactcttaattatcagttcggcgttccgctaaaagatcaattggatagcgataaagtgaaagcggcgcactcgctgatgggcagccgtcttgatttcgttgagcgtaataactttattgttctggaatacaaagaaaaagatccgcttgatgtcaccctgtggttgaaagcggatgccaccaacgagcaccctgagtgcgtcattaaggacactcccgaagcggccgtcggtctggaaaaatgtaagtggaccattaacgcactcattaatcatcattacaaaatcgttgcggcctcctggcaggcgaaaaacaatgccgcccgcacgctggtgatgccggttatcaaagagaatactctgacagagggtaacaataaccactggaacctggtgctgcctgcctggcagtacagttccgatcaagccgaacaagaaaaactcaatacctggcgagtacgtctggcgctggaagatgaaaagggcaaccgacagaactctggcgtggtggaaatcaccgttcagcaggaccgtaaaatagagttgattgttaataacatcgcgaacccagaagagaacaaccacagccacgaagccagcgcacaggcagatggcgttgatggtgtagtgatggatctcgatgtaaccgacagctttggcgataacaccgaccgcaacggcgatgcgttgccggaagataaccttacgcctcagctttacgacgcgcaggacaaacgagtgacgttaaccaacaagccctgctcgaccgataacccctgcgtttttattgccaaacaagataaagaaaagggcactgtcaccctctccagtaccttacctggcacctatcgctggaaagcaaaagccgcgccctacgatgacagtaactatgtggatgtcactttcctcggggcagaaattggtgggctaaatgcttttatctatcgtgtgggggcggctaaacccagcaacctgataggtaaagataaagaaccgttgccgtcaacaacatttatcgatttgttttatggcgcgacaacaataaagacggtgtcttccagcaggtcgaaaaacctgacgaagagatggtgcagtacgactacaagtgggaatttaccggcaagagcatcaatggtaagtgggtgcacaggcgaacactccaatgaggacattgtgattccggccactaaccgtgaagcggcgcaaacctatggcgcacaagcgggagatggcttgcagggatacggtttacgcgtgctgtataccaaaaaa

SEQ ID NO: 98 - DNA, 957 bp, Escherichia coli
atgaagcaggataaaagacgcggtctgacccggatcgcattagcgctggcactggcaggttattgtgtggcacctgtggcgctggctgaagacagcgcctgggtcgacagcggtgaaaccaatattttccaggggaccattccgtggctctattcggaagggggaagtgctacgacagatgccgaccgtgtaacgttgacttctgatctaaaaggcgctcgcccgcaaggcatgaaacggacaagcgtttttactcgggtgataaatattggtgataccgaaggcgacgtggatcttggtggattgggcgataacgcgaaaactatcgatactatccgctggatgagctacaaggatgcgcaggggggggatccaaaagagctggcaacgaaggtgaccagttacactcttaccgatgccgaccgtggtcgctatatcggtattgaaattacgccaaccacgcagaccggtacgccaaacgtcgggactgcgctgcatctttatgacgtttctactgccagcggcggcggaagcgacagcgataacgttgcaccggggccggtggttaaccagaacctgaaagtcgccatctttgttgatggtaccagtatcaaccttatcaacggtagcacaccaatcgaacttggcaaaacctacgtggccaaactgtactcggatgagaacaaaaatggcaagtttgatgcgggtaccgatgctgacgtcaccgccaattatgacttccgttgggtactttctggcagcagccaacagcttggcacttcgggtggcatcgttaactcaagcttcgataataacaatttggtcatccctgcgaccaacgacgaagccagaaccaaccttaacggccctgcgcgcgatggaaaagaggcactttccatcccgaccaacggcgacggggtacagggttacaaacttcacattatttacaaacacaaa

SEQ ID NO: 99 - DNA, 1887 bp, Escherichia coli
atgaagaaagtgctcactctctcactactggctctgtgtgtgtctcatagtgcagtagcagcaaactatacgttcaataacgataatattgccctctcgtttgatgatacaaactcgacgattgtgctgaaggaccgtagaactaaccatccgatcacaccacaggaattgttctttctgacactaccggatgagacaaaaatccacaccgcagatttcaaaatcaagcacatcaaaaaacaggacaatgcgattgtcatcgactttacgcgcccagattttaacgtaacagtgcagttgaaccttgtgaagggaaaatatgccagcatcgactacactattgccgccgttgggcaaccacgagacgtcgccaagattaccttcttcccgaccaaaaaacagtttcaggctccttacgtagacggcgcaatcactagctcaccgatcattgcggactcgttctttatcctgccgaataaaccgatcgtgaatacctacgcctatgaagcaacaaccaatctcaacgtagaactgaaaactccaattcagccagagacgccggttagctttaccacctggttcggtactttcccggaaaccagccagttgcgacgcagtgtgaaccagtttattaatgccgtacgtccacgtccgtacaagccttatttgcattacaacagttggatggatatcggctttttcactccgtacaccgaacaggatgttctgggacgcatggacgaatggaacaaggaattcattagcggccgcggagtggcgttagacgcttttctgctggacgatggctgggacgatcttaccggacgctggttatttggcccggcattcagcaacggttttagcaaagtacgagagaaagccgatagcctgcacagctccgttgggctatggctttcaccgtgggggggttacaataagccgcagcgacgttcgcgtttcgcatgcaaaagagtatgggttcgaaaccgtggacggcaagctggcgctttcgggagcgaactacttaaaaacttcaatgagcagatcattaatcttatcaaaaatgaacacattacctcgtttaaactcgacggaatggggaacgccagttcacatataaagggtagcccgttcgcctcggattttgatgcgtcaatagctctgctgcacaatatgcgcagagcaaacccgaatctatttatcaacctgaccaccggcaccaacgccagcccgtcctggttgttctatgctgattctatctggcgtcagggggatgatataaacctgtatggccccggcacgccggtgcagcagtggataacatatcgtgatgccgagacataccgctctattgtacgtaaaggcccgctattcccgctgaactcgctgatgtaccacgggatagtcagcgccgagaatgcctattacgggttagagaaggtgcaaacggacagcgactttgccgatcaggtctggagctacttcgcgaccggcacccagctgcaggagctgtatattaccccgtccatgctgaacaaggtgaagtgggatacgctggcgaaggctgcaaaatggtcgaaggaaaatgccagcgtgctggttgatacccactggattggcggcgacccaacggcgcttgccgtgtacggctgggcatcctggagcaaagacaaagccattctcggtttgcgcaacccatcggataagccacagacctactatctggatttggcgaaggatttcgaaataccggcaggaaacgcggcgcagtttagtctgaaagcggtatacggcagcaataaaacagtgcccgttgagtataaaaacgcgacggtgattacgttgcagccgctggaaacgctggtgtttgaggcggtgaccattaac

SEQ ID NO: 100 - DNA, 5334 bp, Escherichia coli
atgaacaaaatatttaaagttatctggaatccggcaacaggcagttacaccgttgccagcgaaacggcgaagagccgtggtaaaaaaagcgggcgcagtaagctgttaatttctgcactggttgcgggtgggttgttgtcgtcgtttggggcaagtgcagataattacactgggcagccaactgattatggcgatggctcagcaggtgacggctgggttgctatcggtaaaggggcaaaagcaaatacctttatgaacactagtggcgcgagtacagctttaggatatgacgcgatagccgaaggtgagtacagttctgccatcgggtcaaaaacccttgcaactggtggagcatccatggcgttcggggttagtgcaaaagcaatgggtgacagaagtgtcgcgctaggtgcatcgtcagtagcaaatggcgatcgttcgatggcttttggtcgttacgcaaagacgaatggttttacatctcttgctattggggactcctcccttgccgatggtgaaaaaactattgcgttaggaaatacggctaaagcttacgaaattatgagcatcgccctcggtgataatgccaatgcgtcaaaagagtatgcaatggcgctgggagcaagtagcaaagctggcggtgctgatagcctcgcattcggcagaaaatctacagctaatagcactggctcactggcaataggtgctgacagtagcagttcgaacgataacgccatcgcgatagggaacaaaacgcaagccctgggagtgaattcgatggccctgggtaatgcaagtcaggcatctggcgaatccagtattgcattaggtaacaccagtgaagccagcgaacaaaatgcgattgcgctggggcaaggtagcattgcaagcaaagtgaactcaatcgcgttgggaagtaacagtttgtcctcgggagagaatgccatcgcattgggagagggtagtgccgctggtggcagcaacagccttgctttcggtagccagtccagggcaaacggcaatgattctgtcgccatcggtgtaggggctgcagcagcgaccgacaattctgtcgctatcggcgcaggatcgaccacagatgcaagcaatacggtttcagttggcaacagcgcaacaaaacgcaaaattgttaatatggctgctggtgccataagcaacaccagtaccgatgccatcaacggctcacagctttatacgatcagtgattcagtcgccaagcgactcggaggaggcgctactgtaggcagcgatggcaccgtaaccgcagtaagctacgcgttgagaagcggaacctataataacgtgggtgatgctctgtcaggaatcgacaataataccctacaatggaataaaaccgcgggggcgttcagcgccaatcacggtgcaaatgccaccaacaaaatcactaatgttgctaaaggtacggtttctgcaaccagcaccgatgtagtaaacggctctcaattgtacgacctgcagcaggatgctctgttgtggaacggcacagcattcagtgccgcacacggcaccgaagccaccagcaaaatcactaacgtcaccgctggcaacctgactgccggcagcactgacgccgttaacggctctcagctcaaaaccaccaacgacaacgtgacgaccaacaccaccaacatcgccactaacaccaccaatatcaccaacctgactgacgctgttaacggtctcggtgacgactccctgctgtggaacaaagcagctggcgcattcagcgccgcgcacggcaccgaagccaccagcaaaatcaccaacgtcaccgctggcaacctgactgccggtagcactgacgccgttaacggctcccagctcaaaaccaccaacgacaacgtgacgaccaacaccaccaacatcgccactaacaccaccaatatcaccaacctgactgacgctgttaacggtctcggtgacgactccctgctgtggaacaaaacagctggcgcattcagcgccgcgcacggcactgacgccaccagcaagatcaccaacgtcaccgctggcaacctgactgccggcagcactgacgccgttaacggctcccagctcaaaaccaccaacgacaacgtgacgaccaacaccaccaacatcgccactaacaccaccaatatcaccaacctgactgacgctgttaacggtctcggtgacgactccctgctgtggaacaaaacagctggcgcattcagcgccgcgcacggcactgacgccaccagcaagatcaccaatgtcaaagccggtgacctgacagctggcagcactgacgccgttaacggctctcagctcaaaaccaccaacgataacgtgtcgaccaacaccaccaacatcaccaacctgactgacgctgttaacggtctcggtgacgactccctgctgtggaacaaaacagctggcgcattcagcgccgctcacggcactgacgccaccagcaagatcaccaatgtcaaagccggtgacctgacagctggcagcactgacgccgttaacggctcccagctcaaaaccaccaacgataacgtgtcgaccaacaccaccaacatcactaacctgacggattccgttggcgaccttaaggacgattctctgctgtggaacaaagcggctggcgcattcagcgccgcgcacggtaccgaagctaccagcaagatcaccaacttactggctggcaagatatcttctaacagcactgatgccattaatggctcacaactttatggcgtagcggattcatttacgtcatatcttggtggtggtgctgatatcagcgatacgggtgtattaagtgggccaacctacactattggtggtactgactacactaacgtcggtgatgctctggcagccattaacacatcatttagcacatcactcggcgacgccctactttgggatgcaaccgcaggcaaattcagcgccaaacacggcattaataatgctcccagtgtaatcactgatgttgcaaacggtgcagtctcgtccaccagcagcgacgccattaacggttcacaactttatggtgttagtgactacattgccgatgctctgggcgggaatgctgtggtgaacactgacggcagtatcactacaccaacttatgccatcgctggcggcagttacaacaacgtcggtgacgcgctggaagcgatcgataccacgctggatgatgctctgctgtgggatacaacagccaatggcggtaacggtgcatttagcgccgctcacgggaaagataaaactgccagtgtaatcactaacgtcgctaacggtgcagtctctgccaccagcaacgatgccattaatggctcacagctctatagcactaataagtacatcgctgatgcgctgggtggtgatgcagaagtcaacgctgacggtactatcactgcaccgacttacaccattgcaaataccgattacaacaacgtcggtgaagccctggatgcgctcgataataacgcgctgctgtgggatgaagacgcaggtgcctacaacgccagccatgatggcaatgccagcaaaatcaccaacgttgcggctggtgatctctccacaaccagtaccgatgctgttaacggttcccagttaaacgcaaccaatattctggttacgcaaaatagccaaatgattaaccagcttgctggtaacactagcgaaacctacatcgaggaaaacggtgcgggtattaactatgtacgtaccaacgacagcggcttagcgttcaacgatgccagcgcttcaggtattggcgctacagctgtaggttataacgcagttgcctctcatgccagcagtgtagccatcggtcaggacagcatcagcgaagttgatacgggtatcgctctgggtagcagttccgtttccagccgtgtaatagttaaagggactcgtaacaccagcgtatcggaagaaggtgttgtgattggttatgacaccacggatggcgaactgcttggcgcgttgtcgattggtgatgacggtaaatatcgtcaaatcatcaacgtcgcggatggttctgaagcccatgatgcggtcactgttcgccagttgcaaaacgccattggtgcagtcgcaaccacaccaaccaaatactatcacgccaactcaacggctgaagactcactggcagtcggtgaagactcgctggcaatgggcgcgaaaaccatcgttaatggtaatgcgggtattggtatcggcctgaacacgctggttctggctgatgcgatcaacggtattgctatcggttctaacgcacgcgcaaatcatgccgacagcattgcaatgggtaatggttctcagactacccgtggtgcgcagaccaactacactgcctacaacatggatgcaccgcagaactctgtgggtgagttctctgtcggcagtgaagacggtcaacgtcagatcaccaacgtcgcagcaggttcggcggataccgatgcggttaacgtgggtcagttgaaagtaacggacgcgcaggtttcccagaatacccagagcattactaacctgaacactcaggtcactaatctggatactcgcgtgaccaatatcgaaaacggcattggcgatatcgtaaccaccggtagcactaagtacttcaagaccaacaccgatggcgcagatgccaacgcgcagggtaaagacagtgttgcgattggttctggttccattgctgccgctgacaacagcgtcgcactgggcacgggttccgtagcagacgaagaaaacaccatctctgtgggttcttctaccaaccagcgtcgtatcaccaacgttgctgccggtgttaatgccaccgatgcggttaacgtttcgcaactgaagtcttctgaagcaggcggcgttcgctacgacaccaaagctgatggctctatcgactacagcaacatcactctcggtggcggcaatagcggtacgactcgcatcagcaacgtttctgctggcgtgaacaacaacgacgcagtgaactatgcgcagttgaagcaaagtgtgcaggaaacgaagcaatacaccgatcagcgcatggttgagatggataacaaactgtccaaaactgaaagcaagctgagtggtggtatcgcttctgcaatggcaatgaccggtctgccgcaggcttacacgccgggtgccagcatggcctctattggtggcggtacttacaacggtgaatcggctgttgctttaggtgtgtcgatggtgagcgccaatggtcgttgggtctacaaattacaaggtagtaccaatagccagggtgaatactccgccgcactcggtgccggtattcagtgg

SEQ ID NO: 101 - DNA, 681 bp, Escherichia coli
atgaacctaaagaaaacactgttaagcgtgttaatgatattgcaactttgcttattggtagggtgtgactatattgaaaaagcgagtaaggtcgacgatctcgttacacagcaagagttgcaaaaaagcaaaattgaggcgcttgaaaaacaacaagaactcgacaagcgcaagatagaacactttgaaaaacaacaaactaccatcataaacagtaccaaaacgctcgctggtgtggtgaaggcagttaaaaacaaacaggacgaatttgtctttacagaatttaacccggcacaaacccaatactttattttaaataacggctctgttggtttggcagggaaaatactgtctattgacgcagtagaaaacggcagtgttattcgtatttcactggttaacttattaagtgttcctgtatcaaatatgggtttctacgcaacatgggggggagaaaaacccaccgacatcaacgcattagcaaaatggcagcaattgctatttagtaccgcaatgaactcctccctgaaattattaccaggtcaatggcaagacattaatttgacgctaaaaggtgtctcgcccaacaacctcaaatatctgaaattagccatcaacatggcaaatattcagttcgaccgtcttcaacctgctgaatctccacagcggaaaaacaaaaaa

SEQ ID NO: 102 - DNA, 3327 bp, Escherichia coli
atgaaaagagttgtgcgtcttttgggtgtggggttactgctccttgttgtgttgttgctcattttgtttgttctggctcagaccacaccgctgatatcagcacaggatgagcatgctgtctggcttcgtctgttgataacagcgattgtgatctgtttgctaagtatgtgcatatttttcctcttttctttccggcagaacgaagcctcgacgatatcactatacgctcaaccgactgatataaaggaaataaatacggagcagccgaactatgcatcactgctgacgatatatttacgcgaccgctacggtccgttctggcggcgtaaagtccgcctgctgctggtgaccggcgagcctgaacaggcagaagccatcgcgccggggctgaccgggcaacactggctggaaggcgaccacacggtgctgatatatggcggcaggccaacagcggagcctgatgtcacactgctgaccgccttaaaaaaactgcgccgcagccgtccgctggacggcatcatctgggcgctgacagaagaacagagccgccagacagcgcaactcgacaaaggctggcgcggactgataaacggcggtaagcgactcggttttcaggctccactctatttgtggcaggtctgtgacgacggtgattatcagaccggacgccccctgcaaagcgtcggctgcctgctgccggaacgctgtaccccggaacaactggctgtaatgctggaagcagccgctgacggaacagggcatgtcgcagctactgaccgataccgcatgttttctgctgcgtctggctcatacccttgcagagcggggtattgctcactggcagaccgtcctgaaaccgctgctggcaggcggcgcattttcttccctgcgcctgcgcggcctgatgttcagcccgccgcttgccgccgtgccggaggccagcacctcatgcagtggctgccgtcaccggtctgggcgggcgtgacggtgataacgcgcgcgggcgcacggtgggttttcctgtggctgcgtaccgcactgatgtccgctgtctgcgtgctggtgatatggggggccggaatgacgacctcgttcttcgccaaccgcgctcttgttcaggaaaccggtatccagacggcacgtgcgcttgatacccgcctgccgctggcagaacaactggtggcgctgcataccctgcagggcgaactggaacgcctgcaatatcgtatccgcgaaggtgcgccgtggtatcagcgttttggccttgaacgtaaccaacaactgctcgccgccgcttttcccggctatgcgcaggcggcaaaccggctggtgcgcgacgtggccgttgaccatctgcaacagcaactgaacgcctttgtcgccctgccgcccaacagtcctcagcgtaccgccaccggtgaacaacgctataagcagcttaaggcattgctgatgacttcccgcccggaaaaggccgacgctgcctttttcagtaccacgctgatggcggacggtctgcgctacgagaatatcccggaaggtgtgcggcagagcgtgttgccgtcactgctgaccttctggacggcgaacctgccggaacacccgcagtggaaaacatcgccgccaccggaactgaccggcgcagtgcgtaaaatcctgctgcgccagattggtgtgcgtaatgccgaaaacaccctctaccagaacgtgctgcaacaggtgtcccgcaactacgccgatatgacgctggcggacatgaccggggataccctcaccgaatctcttttcagtacggaacagacggtgccggggatgttcacccgtcaggcgtgggaaggacaggtcagggaagccatcgagcaggtggtgacggcgcggcgcgaggaaatcgactgggtactcagcgaccgccagcaggatacctctgcggatatctcgccggatacgctgcgtaaccgtctcacctcacgctactttaccgactttgccggaagctggctggcgtttctcaacagcattcactggaaaaaggaagactcgctctccggcattctcgaccagctgacactgatggccgatgcccgtcagtcgccactgattgcgctgacggacaccctcgcgtggcaggcggcgacaggcagggaaaaccgtggtctgtcagactcgctggcgaaatcggcacaggaactgtttaacggcaaggagaaaacgccgcagcaatcccgtgaaggtgacgacgtgcctgtcgggccgctggataaaaccttcacgccgctgctgcgtttgctgggcgataaggccggaggcggcgacagccagctgagtctacagacctacctcacccgcgtcacccgcgtgcgcctcaaactgcaacaggtgaccaacgcccccgacccgcaggagatgacccaacaactggcgcagacggtcttacagggtaaaaccgttgacctcaccgacacccgcgactacggacggttaatcgccgccagtctgggcgaagaatggagtggcttcggtcaggcgctgttcgttcgcccggtagagcagtcgtggcggcaggtgctgacgcctgcggcggacagcctgaaccgccagtggcagcgggcgattgtcagccactggaatcaggacttcgctggccgctatccgttcaaagcctcacagaacgatgcctccctccccctgctggcgcagtacctgcgcgatgacgggcgcatcaacctgtttatcgccgccaacctttccggcgtgctgaaacgagagggccgctactgggtggctgacgccatgaacacgcaggggctgacggtcaatccggactttatccgcgccctgaaccgcctgcgcgacgtggccgataccgcctttgccagcggcgatgccgggatacattttgaactgcgggcaaaaccggcgcgtgacgtgatgaagacgcatctggtgattgacgggcaggagctggaatatttcaaccagaaagaacgctggcagcgttttaactggccggatgaacagtggcaacccggcgcatcgctaagctggaccagcacacaggcgatggagcgcatactggcggattaccggggaagctggagtcttattcgcctgctggaacaggcgcaggtgacgccggtggacagcagcacctttaaggtggtgtggaaagcgcaggacggcctgccgctgaattacctgctacgggttgaacagggtaaagggccgctggcgctgctggagctgaaaaacttccgcctgccgggacaggtgtttctgaccggaaaaagtatgaaggatgtggaagagtatggggaagacgccgatgag

SEQ ID NO: 103 - DNA, 534 bp, Escherichia coli
atgtttcctattcgttttaaacgtccggcgttgctctgtatggcgatgctgacggttgttctgagtggctgcggcctgattcagaaagtggtggatgaatcgaaaagcgtggcctcagccgttttctacaaacaaatcaaaatactgcatctcgatttcttctcccgcagcgccctgaatacggatgcggaagatacgccgctttccacgatggtgcatgtctggcaactgaaaacccgcgaagattttgacaaggcggattacgacaccctgtttatgcaggaagagaagacgctggagaaggacgtactggcaaaacacaccgtctgggtaaaaccggaaggcacggcatccctgaatgtgccgctggataaagagacgcagtttgtcgccattattgggcagttttatcaccctgatgaaaaaagcgacagctggcgtctggtgatcaaaagggacgaactggaggccgacaagccgcgctcgattgaactgatgagaagcgacctgcgactgctgcctctcaaggataaa

SEQ ID NO: 104 - DNA, 840 bp, Escherichia coli
atgatttcagggggaaatatgttgaaagaatggatgatatttacgtgcagtttattgactctggctggggcgtcactgcccctcagtggctgtatttccagaggccaggagtctatatccgaaggggcggcatttggggcagggatcctgcgcgaaccgggagcaacaaaaaaagccgacacgaaagacctcaatgtgccaccaccggtttatggtccgccgcaggtgatatttcgcattgatgacaaccgctatttcacgctagaaaattatacccactgcgagaacgggcagacgttttataataataaagcaaaaaacattcatgttaaaatattagacgcttcagggtatttatttaaaggccgcttattctggttatcaacgcgtgatgattttctggcctttcctgccacgttaaataccagacacgcttcctgtatggggtcgaataaaggctgtatgaatgcggtcattgtcactaccgatggtggaaaaagacgcagtggtgtgccatacggcagttatacccagaatccgaccggtgccacgagggattatgacatgctggtgatgaatgacggcttctacctgcttagatatcgggggggacagggcagatttagtccggtgatacttagatggattctcagtactgaagatagctctggtgttgtgcgttcagaagatgcttatgaattgttccgtcccggagaagaggtaccctccaccggtttttataaaatcgacctgtcacgtttttatcccaaaaacaacgttatggaaatgcagtgtgacaggacgctggagccagttcaaccttcagagagtaaaattcaa

SEQ ID NO: 105 - DNA, 1503 bp, Escherichia coli
atggaacacgttagcattaaaacattatatcatctcctgtgctgtatgctgctctttatttccgctatgtgcgctttggcgcaagaacatgagcctatcggggcgcaagatgagcgcctgtcgacattaattcaccaacggatgcaggaggccaaggtcccagccctttccgtaagtgtgaccattaagggggtacgtcagcgatttgtctacggtgttgccgatgtggctagtcagaaagcgaatactctagacacagtttacgagctgggatcgatgagtaaggcgtttaccggacttgtggtgcaaatactgattcaggaaggcagactccggcaaggggatgatatcattacctatctgccggaaatgcgcttgaattatcagggaaaacctgcttccctgaccgtggctgatttcctttatcatacatcaggattgcctttttcaacactggctcggctggaaaaccctatgcctgggagcgctgtggcacagcaactgcgcaacgagaatctgctgtttgcgccgggtgcgaagtttagctatgcctccgccaattatgatgtgttgggcgcggtgattgaaaatgtgacgggaaaaacctttacagaggtcattgcggaacgactcacgcagccgctgggcatgtcggcgactgtggcagttaagggggatgagattattgtcaacaaggcaagcggctataaactgggattcggcaaacccgttctgtttcatgcgcctctggcccggaaccatgttcctgccgcctatatccatagcactctgcctgatatggaaatatggatagacgcctggttgcacagaaaggctttgccggcaacgctgcgtgaggcgatgagtaacagttggcgtggtaatagtgatgttccgcttgccgcagacaatcgtatcctctatgccagcggttggtttatcgaccagaatcaaggcccttacatcagtcacggtgggcagaatccaaacttttcttcttgcattgcgttgcgaccggatcagcagattggcattgttgcgctggcaaatatgaattcgaatctgatactacagctttgcgcggatatcgataattatctgcgcattggcaaatatgctgacggcgctggtgatgcaattacagccaccgatacccttttcgtctacctcacgttgttgctgtgtttttggggggcggtggttgtagtgcgcggtgctttccgtgtttatcgcgcaacggcgcatggccctggaaaacagcagaggttacgtttacgcgtacgtgactatatcatcgccttggcggttcctgggctcgtggccgccatgctctatgtcgcaccgggtatactatctccaggacttgactggcgttttatcttggtatggggtccatcgagcgtgttggcgataccgttcggaattatcctgttagctttcgttctgacattaaatcatcaaattaaacgaattctattacacaacaaggagtgggacgatgag

SEQ ID NO: 106 - DNA, 2046 bp, Escherichia coli
atgaagaacaaatatatcattgctccgggcattgccgtgatgtgttctgcagttatatcatcaggttatgccagttctgataaaaaagaagatacgcttgttgttactgcctccgggttcactcagcagctcagaaatgccccggccagtgtctcagtcattacttcagaacaactgcaaaaaaaaccggtttcagatctggtcgatgcagtaaaagatgttgaagggattagtatcactggtgggaatgaaaaaccggatatcagtatacgtggtctaagtggcgattacacgctgattctggtcgatggacgacgtcagagcggtcgggaatccagaccaaacggcagcggcggttttgaagccggatttatccctcctgtggaagcaattgaacgcattgaagtgatccgtggccctatgtcttccctgtatggttctgatgccatcggaggggtcattaatatcataaccaaaccagttaataaccaaacatgggatggcgtacttggacttggggggattattcaggaacatgggaaatttggtaactcaaccacaaatgacttctatctgtcaggcccattgattaaggataaacttggtcttcagctatatggaggaatgaactatcgcaaggaagatagtatctctcagggaacaccggcaaaagataataagaatataacggcaacgctccagtttactccgactgaaagccagaagtttgtttttgaatatggaaaaaataaccaggtgcatacattaacacctggtgagtctctcgatgcctggactatgcggggaaatcttaaacaaccaaacagtaaaagagaaacgcataattcacgtagtcactgggtagcagcatggaatgcccagggcgaaatactgcatcctgaaattgctgtttatcaggagaaagttattcgtgaggttaaatcaggtaaaaaagataaatataatcattgggatcttaattacgagtcaagaaaaccggaaataaccaacacaatcatagatgcaaaagtgacggcatttctgccggaaaatgtactgaccatcggaggtcaatttcagcatgcagagctccgtgatgactcagccacgggtaaaaaaacgacagaaacacagtctgtttcaattaaacagaaagctgtttttatagaaaatgaatatgcagcaacggattctctcgccctgactggaggactgcgtctcgataatcatgaaatctatggcagttactggaatccaagattgtacgctgtttataacctgaccgataatctcacactcaaaggggggatcgcaaaagcatttcgggctccttcaattcgtgaggtgagtcctggatttggaacactgacgcagggtggtgcctctattatgtatggaaacagggacctgaaaccggagaccagtgtaaccgaagagatcggtattatttatagtaatgatagtggtttttcggcgagcgcgacgctgtttaatactgattttaaaaataagttgaccagttacgatataggtacaaaagatccagtcaccgggttaaacacttttatttatgataatgtaggtgaggcaaatatcagaggggtggagcttgcaactcagattcctgtgtatgataaatggcatgtatctgcaaactatacatttactgactctcgtcgaaaaagtgatgacgaaagtctcaatggcaagtcgctgaaaggggaacctctggaaagaactcccagacatgcagccaatgcaaaactggaatgggattacactcaggatattacattttattcatctctgaattatacgggaaaacaaatctgggcagcacaaagaaatggtgctaaggttccccgcgttcgtaatggattcacatctatggatattggtctaaattaccagattctgccagacacgctgattaattttgccgttcttaacgtcacagacagaaagagcgaggatatcgataccattgatggtaactggcaggtcgatgaaggacgccgttattgggctaatgtaagagtatccttc

SEQ ID NO: 107 - DNA, 492 bp, Escherichia coli
atggggtttagaaaaacaataatcacttcggtaggtttgatatttatttcattctcttttgtggcaaagtgctctcaactcaaaaatttgaataattactcagtgatgctttgtggaaaagtgtcaaataatatcctggatgatattggtggttataaagaaagaaatatattaatgctgcgagctataaaaaaaatcataataatgacaatcgtaaatattatatttttctattcctttcaatcgactgcggatgaaatggttttaataaaaaaatacgggtttgggcttgagagagatatcaaaggaaggccattaatttatcctatcgaaaattatgatgagtgtaagaaaaaatgcaatcatatgaattatatagcggatgtcaatgctcaattagctatgagtaaaaaaaataacaggatttttgctaacataacctttactaacaatagctctaccacgtatttttttctaaatattatctaccta

SEQ ID NO: 108 - DNA, 654 bp, Escherichia coli
atgaatcaaattaaagataataaggtaattatgaaaataaaaaatttaatatcagtcattttactatcaggaggtattatggggactggattgtactcgagcgataaccatcaaaaaatccgcagcaggtttaatatacaggaatcatattgtgccattaagactaatggtgtccttggattcagcaaccgaaaggatgtattgcgagaaaatggtgattcaaccggaaccaccagttccagcactaatgccatgatgctgatggaaaatggtgaaaatgaaatcagtctggagattggggcgttaaggtggttttctgataaacctgccagtaccgaagaacgagggcatttctcccaaaaagcagggtgcagtctggatttggttcgttttgttaagcaggaagaaaccatactttcttcgataaaggtgaccatcaaccagcagggaatacctgaagcgcagccagacagcatgcatcctgttatccgaaaagagattctggctgagcaggcagaacccggatttattgatccagactattttaatgaaacttatttcccgaaagggatgaaggtgtatcaatttacacaaaaggtctcggtggcggggcttcctgatgggcctggacgcagtacgccctttaccggagca

SEQ ID NO: 109 - DNA, 8198 bp, Escherichia coli
atgcatcagcctcccgttcgcttcacttaccgcctgctgagttacctggtcagtgcgattatcgccgggcagccgttgttaccggctgtgggggccgtcatcaccccacaaaacggggccggaatggataaagcggcaaatggtgtgccggtcgtgaacattgccacgccgaacggggccgggatttcgcataaccggtttacggattacaacgtcgggaaggaagggctgattctcaataatgccaccggtaagcttaatccgacgcagcttggtggactgatacagaataacccgaacctgaaagcgggcggggaagcgaagggtatcatcaacgaagtgaccggcggtaagcgttcactgctgcagggctatacggaagtggccggcaaagcggcgaatgtgatggttgccaacccgtatggtatcacctgtgacggctgtggctttatcaacacgccgcacgcgacgctcaccacgggcaaacctgtgatgaatgccgacggcagcctgcaggcgctggaggtgactgaaggcagtatcaccatcaatggcgcgggcctggacggcacccggagcgatgccgtatccattattgcccgtgcaacggaagtgaatgccgcgcttcatgcgaaggatttaactgtcactgcaggcgctaaccgtgtaactgcagatggtcgtgtcagagccctgaagggcgaaggtgatgtgccgaaagttgccgttgataccggcgctctcggtggaatgtacgccaggcgtattcatctgacctccactgaaagtggtgtcggggttaatcttggtaacctttatgcccgcgatggcgatatcaccctggatgccagcggcagactgactgtcaacaacagtctcgccacgggggccgtcactgcaaaaggtcagggcgtcaccttaaccggcgaccataaagcgggaggtaacctgagcgtcagcagccggagagatatcgttctcagcaatggaacgcttaacagcgacaaggacctcagcctgaccgccggcggcagaatcactcaacagaatgaaaaactgactgccggccgggatgtaacgcttgccgcgaaaaacatcacacaggataccgccagccagattaacgcggcccgcgatatcgtgactgtcgccagtgacacgctgacaacacagggacagataaccgccgggcagaatctcacggccagcgccaccacgctgacgcaggacggaatattgctggcgaaaagtcatgcgggactcaatgccggtacgctgaataacagtggcgccgttcagggagctaccctgacgctcggcagtacaacgctcagcaacagtggctccctgctcagtggcggtcccctgaccatgaatacccgcgactttacccagagcggccgcactggcgcgaagggcaaagtggatatcatggccagtgggaaactgaccagtacaggtttgctggtgacgatgcacttggtgctgaaggcgcaggatgtgacacagaacggtgtgctgtccggcggcaaagggctgacggtcagtgcgacgagctccggtaaaaaatcggtcacccacagcgatgctgcgatgacgctgaatgtgacaacagtggcgctggacggggaaaccagtgccggtgacaccctccgggttcaggcagacaaactgagtaccgcagcgggcgcacaacttcagagcggcaaaaatctcagcatcaacgccagagatgcacgtcttgcaggtacgcaggcagcacaacagaccatggtggtgaacgccagtgaaaagctcacccacagcgggaaaagcagtgccccgtcgctcagcctcagtgcgccggaactgaccagcagcggcgtacttgttggttccgccctgaatacacagtcacagaccctgaccaacagcggtctgttgcagggggaggcctcactcaccgttaacacacagaggcttgataatcagcagaacggcacgctgtacagtgctgcagacctgacgctggatataccggacatccgcaacagcgggcttatcaccggtgataatggtttaatgttaaatgctgtctccctcagcaatccgggaaaaatcatcgctgacacgctgagcgtcagggcgaccacgctggatggtgacggcctgttgcagggcgccggtgcactggcgcttgctggcgacaccctctcacagggtagtcacggacgctggctgacggcggacgacctctccctccggggcaaaacactgaataccgcagggaccacgcagggacagaatatcaccgtgcaggcggacagatgggcgaacagtggttccgtgctggcaaccggtaaccttactgcttcggcaaccggtcagttgaccagtaccggcgatatcatgagccagggtgacaccacgctgaaagcagccaccacggacaaccggggcagtctgctttcggccggcacgctctcccttgatggaaattcactggataaccgcggcactgtccagggtaaccatgtcacgattcgccagaacagtgtcaccaacagtggcacgctcaccgggatcgccgcactgacgcttgccgcccgtatggcatcccctcaacctgcgctgatgaataacggaggttcattgctgaccagcggcgatctgacaatcaccgcaggcagtattaccagttccggacactggcagggcaaacgggtgctgatcaccgcagacagtctggcaaacagcggggcgatccaggcggctgacagcctgactgcacgtctgacgggtgagctcgtcagcacagcgggcagcaaagtcacctcgaacggtgaaatggcgctcagtgcactgaatttaagcaacagcggacaatggattgcaaaaaatctgaccctgaaggcgaactcactgaccagtgcgggtgacatcaccggtgtggatactctcacgctcacggtgaatcagacgctgaacaatcaggcgaacggaaaactgctcagtgcaggtgtgctgacgctgaaggcagacagtgtcacaaacgacgggcaattacagggaaatgtcaccaccatcacggcaggacaactcacaaacggcgggcatctgcagggcgaaacgctgacgctgacagcctccggtggcgtgaacaaccgttccggtggtgttctgatgagccggaatgcactgaatgtcagtactgcgaccctgagtaaccagagcacgatacagggtggaggcggggtttccctgaacgccacagaccgtctgcagaacgacggcaaaatcctctccggcagtaacctcacgctgacggcgcaggtgctggcgaacaccggcagcggactggtacaggctgccaccctgctgctggatgtggtgaatactgtcaacggcggacgcgtacttgccaccggcagtgacgttaaaggaaccacgctgaataataccggtacgcttcagggtgcgactctggtgaattaccacacattcagcagcggtaccctgctgggaacctccgggcttggcgtcaagggcagttcactgctgcaaaatggtacagggcggctgtacagtgcaggcaacctgctgcttgacgctcaggacttcagtggtcaggggcaggtggtggccaccggtgatgtcacactgaaactgattgctgccctcacgaatcatggtaccctggccgcagggaaaaccctttccgtcacgtcgcaaaatgccatcaccaacggcggtgtcatgcagggtgatgccatggtgctcggtgccggagaggcattcaccaacaatggactgactgccggtaaaggcaacagtgttttcagcgcacagcgtcttttccttaacgcaccgggttcacttcagggcggtggcgatgtgagtctgaacagccggagtgatatcaccatcagtggttttaccggcacggcaggcagtctgacaatgaatgtggccggtaccctgctgaacagtgcgctgatttatgcggggaataacctgaagctgtttacagaccgtctgcataaccagcatggtgatatcctggccggcaacagtctgtgggtacagaaggatgcttccggcggtgcaaacacagagattatcaataattccgggaatattgagacgcatcagggcgatattgttgtaagaaccgggcatcttctgaaccagcgggagggattttctgccacaacaacaacccggactaacccctcatccattcagggaatgggaaatgctctggttgatattcccctttcccttcttcctgacggcagctatggctatttcacccgtgaagttgaaaatcagcacggtacgccctgcaacgggcacggggcatgcaatatcacaatggatacgctttattattacgcgccgtttgctgacagtgccacacagcgctttctcagcagccagaacatcacaacagtaaccggtgctgataatccggcaggccgcattgcgtcagggcgtaatctttctgctgaggctgaacgactggaaaaccgggcgtcatttatcctggcgaatggggatatcgcactctcgggcagagagttaagcaatcagagctggcagacggggacagagaatgaatatctggtataccgctacgacccgaaaacgttttacggtagctatgcaacaggctctctggataaactgcccctgctgtcaccggaatttgaaaacaataccatcagattttcactggatggccgggaaaaagattacacgcccggtaagacgtattattccgttattcaggcgggcggggatgttaagacccgttttaccagcagtatcaataacggaacaaccactgcacatgcaggtagtgtcagtccggtggtctctgcacctgtactgaatacgttaagtcagcagaccggcggagacagtctgacacagacagcgctgcagcagtatgagccggtggtggttggctctccgcaatggcacgatgaactggcaggtgccctgaaaaatattgccggaggttcgccactgaccggtcagaccggtatcagtgatgactggccactgccttccggcaacaatggatacctggttccgtccacggacccggacagtccgtatctgattacggtgaacccgaaactggatggtctcggacaggtggacagccatttgtttgccggactgtatgagcttcttggagcgaagccgggtcaggcgccacgtgaaacggctccgtcgtataccgatgaaaaacagtttctgggctcatcgtattttcttgaccgcctcgggctgaaaccggaaaaagattatcgtttcctgggggatgcggtctttgatacccggtatgtcagtaacgcggtgctgagccggacgggttcacgttatctcaacggactgggttcagacacggaacagatgcggtatctgatggataacgcggccagacaacagaaaggactgggattagagtttggtgtggcgctgacagctgaacagattgctcagcttgacggcagcatactgtggtgggagtcagtcaccatcaacggacaaacagtcatggtcccgaaactgtatctgtcgccggaagatatcaccctgcataacggcagcgttatcagcgggaacaacgtgcaacttgcgggcggcaatatcaccaacagcggcggcagcatcaacgcacagaacgacctctcgctcgacagttccggctatatcgacaacctgaatgcggggctgataagcgcgggcggtagcctggacctgagcgccatcggggatatcagcaatatcagctcagtcatcagcggtaaaaccgtacaactggaaagcgtgagtggcaacatcagcaatatcacccggcgtcagcaatggaatgcgggcagtgacagccaatatggtggtgtgcatctcagcggtacggacaccggtccggttgcgaccattaaaggcactgattcactttcgctggatgcagggaaaaacattgatattaccggggcaacggtctcgtccggtggagaccttggaatgtctgcgggtaatgatatcaacattgccgcaaacctgataagtgggagcaaaagtcagtccggtttctggcacactgatgacaacagttcatcatccaccacctcacagggcagcagcatcagcgccggcggtaacctggcgatggctgcaggccataatctggatgtcacggcatcctctgtttctgccgggcacagcgccctgctttcttgcaggtcacgacctagtcttgaatgcagtcagggaaaagcaaaaacaagtcgcaacggcaggtcagaaagtcatgaaagccacgcagctgtgtccacggtgacagcgggcgataacttcctccttgttgccggtcgtgatattgccagtcaggctgccggtatggctgcggaaaataacgtggtcatccggggcggacgtgatgtgaacctggtggcagagtctgccggcgcaggcgacagctatacgtcgaagaaaaagaaagagattaacgagacagtccgtcagcagggaacggaaatcgccagcggtggtgacaccaccgtcaacgcaggacgggatatcaccgctgttgcgtcatccgttaccgcaaccggcaatatcagcgtgaatgccggtcgtgatgttgccctgaccacggcgacagaaagtgactatcactatctggaaacgaagaaaaaaagcggaggttttctcagtaagaaaaccacccgcaccatcagtgaggacagtgccacccgtgaagcaggctccctgctgtcggggaaccgcgtgaccgttaacgccggtgataacctgacggtagagggttcggatgtggtggctgaccgggatgtgtcactggcggcgggtaaccatgttgatgttcttgctgccaccagtacagatacgtcctggcgctttaaggaaacgaagaaatccggtctgatgggtaccggcggtattggtttcaccattggcagcagtaagacaacgcacgaccggcgcgaggccgggacaacgcagagtcagagtgccagcaccatcggctccactgccggtaatgtcagtattaccgcgggcaaacaggctcatatcagcggttcggatgtgattgcgaaccgggatatcagcattaccggtgacagtgtggtggttgacccggggcatgaccgtcgtactgtggacgaaaaatttgagcagaagaaaagcgggctgacggttgccctttccggcacggtgggcagtgccatcaataatgcggttaccagtgcacaggagacgaaggagagcagtgacagccgtctgaaagccctgcaggccacaaagacagcgctgtctggtgtgcaggccggacaggctgcgacaatggcctccgcaaccggtgacccgaatgcgggagtcagcctgtcgctcaccacccagaaatcgaaatcacaacaacattctgaaagtgacacagtatccggcagtacgctgaatgccgggaataatctgtctgttgtcgcaaccggcaaaaacaggggcgataaccgcggagatattgtgattgcaggaagccagcttaaggccggtggtaacacaagcctggatgccgcgaatgatattctgttgagtggcgccgcaaacacacaaaaaacaacgggcaggaacagcagcagtggcggtggcgtgggtgtcagtatcggtgcaggtaaaggtgccggtatcagcgcctttgccagcgttaatgcggcaaaaggcagggagaaaggtaacggtactactaccgacaaaaccgtcaccatcaacagtggtcgggatacggtactgaacggtgctcaggtcaacggcaacaggattatcgccgatgtgggccacgacctgctgataagcagccagcaggacaccagtaagtacgacagtaaacagaccagcgtggctgccggcggcagttttacctttggctccatgaccggctcaggttacatcgctgcctcccgggataagatgaagagccgctttgactccgttgctgaacaaaccggaatgtttgcccgggtgatggtggcttcgacatcacagtgggtaaacatacccaactggatggtgcggtcattgcctcactgccacaccggagaaaaaccacctggataccggacgctgggtttagtgactttacaacgaagcgggattataaagtcaagtcacaggtggaatcagtctga

SEQ ID NO: 110 - DNA, 963 bp, Escherichia coli
atgatgttgaagaaaacgatatttatattaacgttattctctggcaacgtaattgctgcaactgtagaattaggttttgaaaatgagcaatataattatgcttatcgttctgcagatgtcttcatgccgtatattaagagtaatttcaaccctgttactgattctgctttgaatgtgtcactcacctatatgtatcaggatcaatatgggaaaaaacataaaaaaacatctgaggacagatttaaaaccaatcgcgatcgcatagagctctatcttaaaggttatactttaaataggggagcatattctttttctccttccgcaggtttccgttatgagtcatgggatgtaaactacgataatccgaaaaagcaggataagtggaaactggaactacgcttttatcctaatatgacttataaactcaatgaccagttaagcctatatatgaatggttttgttgcccctgtattttttaaaacacaacaagagtcgagaaaagataacaattatgtaaagggtaagttaggggcgaaacgttataacaacgattattatcaggaactccagattctgggtgtcagatataaatttaataatgataatacgctctgggcatcagtctataatgaaagaaaatataatcaacattcctcaaaatatgatcgctggcaattgcgtggaggctatgattttaaagttacagaggagtttgttttgagtccattcataagatatgacctctcttatagagaaaaaaacctcgaaagcacaagtaataatggtttatcaaaaaataataaagaaattcgaactggagccagcttttcctataaaattatcccttctgtaaaactggtaggagaaatatacaggcaaacaaccaacattgaaaactattatggagagcattctgaagacaaaaaccgcatgttctacaaacttggtataaacaaaacattt

SEQ ID NO: 111 - DNA, 1761 bp, Escherichia coli
atgcagcaccggcagaaaaacattctgacgaaaacgtcccttttatcccgtgcgttgtctgtcccctgttgtgatatgttccggcgcggctctccgtggatatgctatctctccctctccgttttttctggttgtttcatccccgcattttcgtctccggcagccatgctgtctccgggtgaccgcagtgcaattcagcagcaacagcagcagttgctggatgaaaaccagcgtcagcgtgatgcgctggagcgcccgctgaccatcacgccgtctccggaaacgtctgccggtactgaaggtccctgctttacggtgtcaagcattgttgtcagtggggccacccgactgacgtctgcagaaaccgacagactggtgccgtgggtgaatcagtgtctgaatatcacggggctgaccgcggtcacggatgccgtgacggacggctatatacgccggggatatatcaccagccgggcctttctgacagagcaggacctttcagggggcgtactgcacataacggtcatggaaggcaggctgcagcaaatccgggcggaaggcgctgaccttcctgcccgcaccctgaagatggttttcccgggaatggaggggaaggttctgaacctgcgggatattgagcaggggatggagcagattaatcgtctgcgtacggagccggtacagattgaaatatcgcccggtgaccgtgagggatggtcggtggtgacactgacggcattgccggaatggcctgtcacagggagcgtgggcatcgacaacagcgggcagaagagtaccggtacggggcagttaaatggtgtcctttcctttaataatcctctggggctggctgacaactggtttgtcagcgggggacggagcagtgacttttcggtgtcacatgatgcgaggaattttgccgccggtgtcagtctgccgtatggctataccctggtggattacacgtattcatggagtgactacctcagcaccattgataaccggggctggcggtggcgttccacgggagacctgcagactcaccggctgggactgtcgcatgtcctgttccgtaacggggacatgaagacagcactgaccggaggtctgcagcaccgcattattcacaattatctggatgatgttctgcttcagggcagcagccgtaaactcacttcattttctgtcgggctgaatcacacccacaagtttctggggggggtcggaacactgaatccggtattcacacgggggatgccctggttcggcgcagaaagcgaccacgggaaaaggggagacctgcccgtaaatcagttccggaaatggtcggtgagtgccagttttcagcgccccgtcacggacagggtgtggtggctgaccagcgcttatgcccagtggtcaccggaccgtcttcatggtgtggaacaactgagcctcgggggtgagagttcagtgcgtggctttaaggatcagtatatctccggtaataacggcggttatctgcggaatgagctgtcctggtctctgttctccctgccatatgtgggaactgtccgtgcagtggctgcactggacggcggctggctgcactctgacagcgatgacccgtactcgtccggcacgctgtggggtgctgctgccgggctcagcaccaccagtggccatgtttccggttcgttcactgccggactgcctctggtttacccggactggcttgcccctgaccatctcacggtttactggcgcgttgccgtcgcgttt

SEQ ID NO: 112 - DNA, 2220 bp, Escherichia coli
atgaataagcacacactattactgactgttctttttctgaatttgatttgtactcccgtttttgctcaaaactggcaggtggcgacgtttggtcagtctacggatctcaacttttcatcgctgatagattcggccaagatcggacggaataatgcctggcttgcaggaaacaataattttcttgaagctggaaaattttacactttaccaacagatttttttattgaaagccgtgggggaaaaattgctaactcccatgacggtatgaccgtcttttatactattgttccggttactcagacattccgactggaggctgatttgacattagaacagattggtccggaggtgaatggaaaatcaccagcgggacaggagggagctggattgtttgtcagagatattatcggtcctcagcgacaggaacctcagtcagctggaacagaagaatatccccaggcctctaatatattgatgaatgcctttattacacagaataaaaagaatgataacttagtacagattacttcaattgttcgtgaaggagtaataaaaacatggggtaatgaaggtattacaattaagaaacagccgatcattgagaatataaactttacgcaaaaaagaaatattcatatgacgatcgagcgactaccagagaagttcatcctgaccgcttttgataccgatcgtaaagaaaatcagtcatggcaattttctgattactcaggctttatgaatcaactggataataatagtttagctattggtttttttgccgcacgaaatgcgaaactaagggtgaaaaatgcatcatttaaaccgggcaagccactggttgattacaaacaattaacttcacgtcaattcagtcgtgtccggcataaagcccctgaactttttcttgcttcacctcaatccgttgtaagaaactcaacaactcttcaatttttggccaatcaggctggaatagtcagtattgataatgataagcagactaagcaggtgcaggcgggtgaactggtacagtttccagttactttgcaaaaaaaacataatgacttcaccgtcaactttaacgttgatgggaatatatcaaaaaaagctatacgcatagagcaggttaaatcaaacctgactgatccttatgagatttacgtatgtagtgattgtcgacagggggccagaggcagcaaaaatgaccctgtagatttacagacagccgtaaaatttgtcgcacccggcggtaatatataccttaacgatggtcaatatcatggaattaccttagatcgggaattaagtggaatacctggcaagtataaaacaatttctgccattaatccacataaagccatttttataaacaagacattcaatctggatgcaagttactggcatctaaaatccgtggtctttgacggcaatgtggataatggaaataataaaccagcatatttgcgtatagctggtagctataatattattgagcatgtgatagccagaaataatgatgatacgggaatttctatttcagcgaaagataaaaaccgttttttctggccagctcataacttagttttaaactcagattcatataataatcttgatttatccgggattaatgccgatggttttgctgcaaaattaggtgtcggaccgggaaacatttttcgaggatgcattgcacataataatgcagatgatggttgggacctatttaacaaaattgaagatggtccaaatgcatctgttactattgagaattctgtagcctatgaaaatggcctgccatacaataaagcggatatcctaaaagggagtattggcaatggcggtgaaggtcaacccagtaaatcacaagttattaattccattgctattaataataatatggatggattcactgataattttaatactgggtcattgatagttagaaataatatagcaatgaacaatgcacgctataattatattttaagaactaacccatataaattcccatcatctatcctttttgataataattattcaatcagagatgattgggaaaataaaataaaagacttcttaggtgatacagttaacagtgtgaattataaattgcttgtttcacatgaaacaggaccggtacaaaaagatttatttttcacacgagatgatagtggaaatattatctatcctgatttttttcttaatatcattaataaatttaat

SEQ ID NO: 113 - DNA, 408 bp, Escherichia coli
atgaaaacttttatcaaaactttactcgttgctgtaactattctgttctctgtcttcgctacggcgaaacaagtaaaactgccaaacaacatcaaatacgttaatactacagaggcgttttcctgtactgagattgacggtatgaattgccagacgaagaatccgtttaactataaagataacagctatgttttcgtgcttgaacgtggtggtgcctggtgttacgactacactgtctcggtacttaacctgaaaaccgggaaagcacagatgctcgaatacaaagacaaccagctgtgctcaggtagcaacaaaccgttcttcgaaatcaaaaatggcgtaccgacggtaggagtcatcgacacatccggaaaacctgtcgttgtggctctggacaaacttaaaacc

SEQ ID NO: 114 - DNA, 675 bp, Escherichia coli
atgcaattacctgtaaagttattaatgagccttatatctctggtcagcgttattgcacgtgccgggaaatataaaaattacatccgggatgaaataaaatactggcgatatacatcatacaaggggggggaatttccggaaggtttcactgatgagaaattttccagcgccatttacaacggaagaatatttacaatgaaacgtttacataccctgatgttatttctggcggttctgtttactggctttaacgtggaagcagcgagcgtgaaacaagcgctcagctgcgacccaaacgcccgggctgaacaacctggagcgtgtccaacaacgtacgagttgtacgaaggtgacgctgcctacaaagctgcgcttgacaaagcattaaaaccggtcggactgagcggcatgttcggtaaaggcgggtatatggatggccctggcggaaacgtaacgccagtaaccattaacggtacagtctggctccagggcgacggttgcaaagccaatacctgcggctgggactttatcgtaacactctataacccaaaaacccatgaagtcgttggctaccgctactttggtttagatgacccggcctacctggtttggttcggcgaaattggcgtgcatgaattcgcgtatctggtgaaaaactacgtagctgcggttaac

SEQ ID NO: 115 - DNA, 2163 bp, Escherichia coli
atgaaaactcaaataactttcgctgcgcttttgccagcattagcgtctttcataccgcttcatgctcatgcctcgtctacttctgaagatgaaatgattgtcacgggcaacaccgccgccgacaccaccgattctgccgccggtgccggtttcaaaacgaacgatatagatgtcggcccgctgggaacgaaatcctggatcgaaacaccatattccagcaccactgttactaaagagatgattgaaaatcagcaggcgcaaagcgtcagcgagatgctgaaatactctcccagtacgcaaatgcaggcgcgcggtggaatggatgtcgggcgtccgcaaagtcgggggatgcagggcagcgtggtggccaacagccgtctggacgggctgaatatcgtttcaacaaccgcgtttccggtggaaatgcttgagcgcatggatgtgcttaacagtttgaccggcgcgctgtacggcccggcgagcccagcagggcagtttaatttcgtggcgaagcgcccaaccgaagagacgctgcgtaaagtgacgctgggctatcaaagccgcagtgcgtttaccggccatgccgatctgggtggccattttgatgaaaacaaacggtttggctatcgcgtgaacctgcttgatcaggaaggggaaggcaatgtggatgacagcacgctgcgtcgcaaactcgtttccgttgcgctcgactggaatattcagccgggcactcagctacagctcgacgccagccattacgaatttatccagaaaggctatgtcggtagctttaactatgggccgaacgtcaaactgccgtctgcgccgaatccgaaggacaaaaatctggcgctcagcactgcgggcaacgacctcactaccgataccatcagcactcgcctgatccactactttaacgacgactggtccatgaacgctggcgtgggctggcagcaggctgaccgcgcgatgcgtagtgtttccagtaaaatactcaacaatcagggcgatatctctcgttcgatgaaggattccaccgctgccggacgttttcgcgtcctgagcaacaccgccgggctgaatggtcatattgataccggctctatcggccacgatctgtcactttctaccacgggatatgtctggtcgctttatagtgccaaaggaacaggttccagctatagctggggtacaacaaatatgtatcacccggatgcgatagatgagcagggcgatggcaaaatccgcaccggcgggccgcgataccgctccagcgtaaatactcagcagagcgttacgctcggcgatacggtgacatttacgccgcagtggtcggcaatgttctatctcagccagagctggctgcagactaaaaactacgataagcacggtaatcaaacgaaccaggttgatgaaaatggtttaagtccgaacgccgcgctgatgtataaaattacccctaacacaatggcctacgttagctatgccgattcgctggagcagggcggtaccgcaccgacggatgagagcgtaaaaaatgccggtcaaacgctaaacccgtatcgcagcaagcagtatgaagtggggctaaaatcggacatcggcgagatgaatctaggcgccgcgctgttccgactggaacgtccgtttgcctatcttgatacggataacgtgtataaagagcagggtaaccaggttaacaacggccttgagttaaccgctgccgggaatgtgtggcaggggctgaatatttacagcggcgtgaccttcctcgacccgaaactgaaagatacggcgaatgcctcaaccagcaataaacaggttgtcggcgtgccgaaagtgcaggccaatctgttggcggaatacagtttgccgtccataccggaatgggtttacagcgctaacgtccattatacgggcaaacgcgcggcgaacgataccaacacctcttacgccagcagctataccacatgggatttgggaacgcgttacaccacgaaagtgagcaacgtcccaaccactttccgcgtggtggtaaacaacgtgtttgataaacattactgggcttctatcttcccatcgggtaccgatggcgataacggttccccaagtgcgtttatcggcggcggccgcgaagtgcgtgcatccgtcaccttcgatttc

SEQ ID NO: 116 - DNA, 2007 bp, Escherichia coli
atgaaaaacataacgctgtggcagcgtttaagacaggtcagtatcagtaccagcttacgttgcgcatttctgatgggggcacttctgaccctgattgtcagtagtgtcagtctgtattcatggcatgaacaaagctcacaaattcgttactcgctggataagtattttccccgtattcactctgctttccttattgaagggaacctgaatctggtggtagaccagctaaatgaatttttgcaggctcccaacaccacggtgcgattgcaacttcgtacccagattattcagcatctcgacaccatagaacggcttagtaggggactgtcatcccgggaacgccaacaactgacggtcattttgcaggacagtcgatcactgttatccgagttggatcgtgcgctttacaacatgtttttactacgggaaaaggtgagtgagctatcagcgcggattgactggttacacgatgattttactaccgagcttaattctttagtgcaggatttcacctggcagcagggaacgctgctggatcaaatcgcctcccgacagggcgatacggcgcaatacctgaagcgatctcgtgaagtgcaaaatgaacagcagcaggtttataccctggcacgcattgaaaatcagattgttgacgatctgcgtgacagactcaatgagctcaaatcaggacgtgatgacgacatacaggtggaaactcatctccgttattttgaaaatctgaaaaaaacggcagatgaaaatatacgtatgctggatgactggcctggcaccattaccctgaggcagaccatcgatgaattgctggatatgggaatcgtaaaaaacaaaatgccggatacgatgcgtgaatatgtcgccgcccaaaaagccttagaggatgccagtcgcaccagggaagcgacacagggtcgcttcagaacgttactggaagcgcagcttggcagtactcatcaacaaatgcagatgtttaatcaacgaatggaacaaattgttcacgttagcggtgggctgatcctggtggcgacagcactggcgttactgcttgcatgggtattcaaccattattttatccgctcacggttggtgaaacgctttaccctactgaatcaggccgttgtgcaaattggtctgggaggcacggaaacgactattccagtttatgggaatgatgaactggggagaattgcaggattattacgccatactctcggccaactcaatgtgcaaaaacagcaacttgaacaagaaattaccgatcgtaaggtgatagaagccgatctgcgtgccacccaggacgaactgattcagacagcaaagttggcggtagtcgggcaaacgatgaccacgctggcccacgagatcaatcagccgctaaatgcgctgtcaatgtatctgtttacagcccgcagggccattgaacagacccagaaagaacaggccagcatgatgcttggtaaagccgaaggggtgattagtcgtattgacgccattatccgttcactacggcagtttacccggcgcgccgaactggaaacatcactccatgccgttgatttagcacagatgttcagtgcggcctgggaacttctggccatgcgtcatcgctctctgcaagctacgcttgttctgccgcaaggtacagccacagtttcaggtgatgaggtcagaacccagcaggtactggttaacgtactggcgaatgcgcttgatgtttgtgggcaaggcgctgtcattaccgttaactggcaaatgcagggtaaaacgctgaacgtattcattggcgataatggcccgggctggcctgaggcattgttgccttcgttattgaagccgtttaccaccagtaaagaagtaggactgggtattggtctttcaatttgtgtgtcgttgatggagcaaatgaaaggggaattgcggctggcatcaacgatgaccaggaatgcctgtgtggtactgcaattcagactaacggatgtggaagatgctaag

SEQ ID NO: 117 - DNA, 2259 bp, Escherichia coli
atgaacgttataaaactggctatcggctcaggcatattattgctcagctgcggtgcttactcacaatccatcagtgaaaaaactaattccgacaaaaaaggagcggcagaattcagtccgctcagcgtttctgtcgggaagacgaccagtgagcaggaagctctcgagaaaacaggcgcgaccagttcccggacaacggacaaaaacctgcaatcacttgacgcaacagtgcgtagtatgcctggtacttatactcaaatagatcctggtcagggagcaatcagtgtgaatattcgaggcatgagcggatttggtcgtgtaaacactatggtcgatggtattacccagagtttttacggaacctctacctccggaacaacgacgcatggttcaactaacaatatggctggcgtacttatagatcctaacttactggtagcagttgatgttacacgcggtgacagcagtggctctgaagggatcaacgcccttgccggtagtgcaaatatgcgtactattggcgttgacgatgtaatatttaacggtaatacatatggccttcgttcacgtttctctgtcggtagtaatgggctgggacgcagcggaatgatcgcccttggtggaaaaagcgacgcttttacggatacgggaagcattggcgttatggctgctgtgagcggcagttctgtgtactctaatttctcaaatggttctggaattaacagcaaagagtttggttatgataaatatatgaagcagaaccccaaatcccaactgtataaaatggatatcagaccagacgaatttaacagcttcgaactttccgctcgaacctatgaaaataaatttacacgtcgtgatataaccagtgacgactattacattaaatatcattacacccctttttctgaattaattgactttaacgtaacggccagtaccagtcgcggtaatcaaaagtatcgtgatggctcgctgtatactttctacaaaacctcagcgcaaaatcgttctgacgcgctggatatcaacaataccagccggttcactgtcgcggacaatgacctggagtttatgctgggcagcaaactgatgcgtacccgctatgaccggaccattcactcagcggcgggcgacccgaaagcgaatcaggaatcgatcgagaacaatccgttcgcaccctccggccagcaggatatttcagcgctgtataccgggctgaaggttacgcgcggcatctgggaggcagatttcaatctcaactacacacgtaacaggatcacagggtacaagcccgcctgcgattcacgcgttatctgcgtgccacagggtagctacgatattgacgataaagagggtggcttcaacccttcagttcagctttctgctcaggtaacaccatggcttcagccgttcattggctacagcaaatccatgcgcgccccgaacatccaggagatgttcttctctaattcaggaggcgcatccatgaacccattcctgaagcctgaacgtgcagaaacctggcaggcgggttttaacattgataccagagatttactggtcgaacaggatgccctgcgctttaaggctctggcgtaccgcagcaggatccagaactacatctacagcgagtcttatctggtttgttctggaggtcgtaaatgcagtctgcctgaggtgattggcaatggctgggagggcattagcgatgaatacagcgacaatatgtacatctacgttaactcggcaagcgacgttatcgcaaagggcttcgaactggagatggattatgatgcaggttttgcttttggccgactctctttcagccagcagcaaacagaccagccaacctccatcgccagcacccactttggcgcaggggatataaccgaactgcccagaaaatacatgacgctggatactggtgttcgcttcttcgataacgcgttgaccctgggcactatcataaaatacacaggcaaggctcgtcgcctgtcgcctgattttgagcaggacgaacataccggcgcaataatcaaacaggatttgccgcagatcccaacgattatcgatctctatggtacttacgagtacaaccgcaacctgacactgaaactttcggtacaaaacctgatgaacagagattattcggaggcgctgaataagctcaacatgatgccaggtcttggtgacgagacccacccagccaattccgcgcgtggcagaacatggatatttggcggggacattcgtttc

SEQ ID NO: 118 - DNA, 399 bp, Escherichia coli
atgtcttcgaaaacaaaatgctggctatggatgttactggtcatcctttctgaaacctctgcaacatccacacttaaaatgttcgataacagtgaggggatgacaaaaacgctgctgctggccctaatcgtcgtactgtattgcatttgttactactcgctttcacgggcagtaaaagatatccccgttggtctggcttacgccacatggtccggtactggcattttgatggtttcaacccttgggattttattttacggtcaacacccggataccgccgccattattggtatggtcatcatagccagcggtattatcattatgaatctgttctcaaaaatgggcagtgaagaggcggaagaaactccagttaccaacctcgataaaaaaatcgctaac

SEQ ID NO: 119 - DNA, 858 bp, Escherichia coli
atgtatataaaaaagcactggatagctttatccattctattaataccttgcattggaaacgctcaggaaattaaaattgatgaaagctggttacatcaaagcttgaatgtcattggtcgcacagactctcgctttggcccaagactgactaacgacctctaccctgaatatactgtagcaggaagaaaagactggtttgatttttatggttatgttgatctaccgaaattctttggcgtcggcagtcactatgatgttgggatctgggatgagggctcaccactatttacggaaatagaacctcggttttccattgacaaattgaccggattaaatcttgcgttcggcccatttaaagaatggttcattgcaaacaactatgtctatgatatgggtgacaaccagtcatcccggcaaagtacatggtatatggggcttggtacagatatcgacacgggtctaccaattaagctttctgccaatatatacgccaagtatcagtggcaaaactatggtgccgctaatgaaaatgaatgggacggatatcgattcaaaataaaatatagcatccctcttacaaatttattcggaggacgattggtatacaatagttttactaactttgattttggctccgatcttgcggacaagtcacacaataataaacgaaccagtaatgctattgcttcaagccatatcctttcccttctatatgaacactggaaatttgcatttacactacgttattttcacaacggtggacaatggaatgcgggagagaaggttaacttcggagatggtccatttgaattaaaaaatacaggatggggaacctatactactattggttatcaattt

SEQ ID NO: 120 - DNA, 516 bp, Escherichia coli
atgagaatcgcaccgcgtaccttctttgctatttccgccctggcgtttattgtcgcctccggatttagtttctggcggttgtcccctgctgaaaatacagggattatgagttgttcaacaaaaggcatcatgcgttttgagaatatggaaaaggagaacgttaacggtaatattcactttaactttggcagccagggtaaaggttcgatggtgctcgaaggctacacggactctgccgctggctggctgtacctgcaacgctatgtcaaatttacctataccagtaaacgtgtttccgccacggaacgccattaccgcatcagccagtgggaatccagcgcctcatcgatagatgaatcaccagatgtgatttttgactactttatgcgtgaaatgtctgacagccatgacgggctgttcctcaacgcccagaagctgaacgataaagcgattttgctcagttctattaattcaccgctttggatctgtacccttaaatctggcagcaaattagac

SEQ ID NO: 121 - DNA, 546 bp, Escherichia coli
atgaaaataaaagttatagcattggctacatttgtttctgctgtgtttgctggttcagctatggcctatgacggaacaattacgtttaccggtaaagttgtagctcagacctgcacagttaatacaagcgacaaagacttagcagtaactttacccactgttgccacttcatctctaaaagacaatgctgctacgtcagggctgacaccctttgccattcgtttaactggttgtgcaactggtatgaatagtgctcagaatgttaaagcgtactttgagccttcaagtaacattgacttagctacacataatttaaaaaatactgctactccaactaaagcggataatgtacagattcagttgctaaatagtaatggaacttcaactattcttttgggggaagcggataatgggcaagatgtccagtctgagacaatcggatctgatggaagtgccacattgcgttatatggcccagtattatgcaacaggacaatctaccgcaggggatgtaaaagcgacggtccattataccattgcctacgaa

SEQ ID NO: 122 - DNA, 1077 bp, Escherichia coli
atgaaaagaatctttttcataccattgtttttaattttactccctaagctggcggtagcgggtccggatgattatgtgccttcgcagatagcggttaatacatccacattgccaggtgttgtgattggtcctgctgatgcccatacctatccccgggtgataggagagctggcgggaacaagtaaccagtatgtttttaatggcggtgccatcgctctgatgcgtggaaagtttacacccgcactgcctaaaattggaagtattacggtatactttccatcaaggaaacagcgtgattcatctgattttgatatctatgatattggtgtatccggactgggtattattattggcatggcgggctattggcccgcaacgcctctggtccccataaatagctcaggtatatatattgaccctgtaggtgccaatacaaaccccaatacttataacggtgcgacagcaagcttcggagctcgtttgtttgttgcttttgtcgcaacgggaagattacccaatggatatataacaatacccaccaggcagcttggtactattttgttggaagcaaaacgtacaagtttaaataataaaggactgacagcacctgttatgttaaatggtgggcgcattcaggtacagagtcagacatgtaccatggggcaaaaaaactatgtggtgccattaaataccgtatatcaatcacagttcacatctttgtataaagaaatacagggaggtaaaattgacatacacctacaatgcccggatggaattgatgtttatgctacattgacagatgcatcacagccagtgaacagaacagatatattgaccttaagcagtgaatccactgcaaaaggatttggcatcaggctatataaagacagtgatgtaactgccatcagctatggtgaagactcccctgtgaaaggaaatggcagtcaatggcacttctccgattacaggggagaggtaaatccacatatcaatttaagagccaattatataaaaattgctgatgcaactacacctggaagtgtgaaggctattgcaactattactttctcatatcaa

SEQ ID NO: 123 - DNA, 2532 bp, Escherichia coli
atgaacgctaataatctgtcatgcctgatttactgtcgttgttctcttctgctttttgctgcattagggttaacagtaacaaaccattcatttgctgctgaagaggctgagtttgattctgagtttttgcatttggataaagggataaatgctattgatatccgccgctttagtcatggtaaccctgtgcctgagggcaggtattattctgatatttatgttaataatgtatggaaggggaaggctgatttgcagtatttacgtactgccaataccggtgctccgacgttatgcctgacgcctgagctgctttcattgattgatttagtcaaagatactatgtcgggaaacacctcctgctttccggcgtcaacagggctttcttcagccagaattaattttgacttatcgactttaaggttgaatatcgaaatccctcaggcactgctgaatacacgtccaagaggatatatttcccctgctcagtggcaaagtggtgttcctgcagcatttataaactatgatgctaactattaccagtatagctcttccgggacgagtaacgaacagacttatctgggattaaaagctggattcaatttgtggggatgggctttgcgccaccgtggcagtgagagctggaataatagctatcctgccggatatcagaatatagaaacaagtataatgcatgaccttgccccattgagagcacaattcacattaggggatttttatacgaatggtgagctaatggatagcctcagtttgcggggagtcaggttagcatcggatgaacgaatgttacccggctctttacgtggctatgctcctgctgtccggggaatagctaacagtaatgctaaagtaaccatttatcaaaatgctcatatcctctatgaaacgacggtgccagccggaccatttgtcatcaatgatttatatcccagtggatatgctggtgaccttctcgttaagataacagagtctaatggccagacacgaatgttcacggttccttttgcggccgttgctcaactcattcgtcccggatttagtcgctggcaaatgtcagtgggaaagtatcgttatgcgaataaaacatataatgatttaatagcacaaggcacctatcaatacggcctgacgaatgatattactttaaacagtggtcttaccacagcttcaggatatacagcggggttagctggcctggcctttaatacccctctgggtgctatagcatctgacattacattgtccagaacagcattcaggtattccggtgtaacgcgtaaaggttatagtctgcactcaagttatagcatcaatattccagcctcaaacacaaatataactctggcggcttatcgttattcatcaaaagatttttatcatctgaaggatgcgctatcagctaatcacaacgcgtttattgatgatgtttctgtaaaaagtacagcgttttatcgtcccaggaatcaattccagatttcaatcaaccaggaattaggtgaaaaatggggtgggatgtatttaacaggaacaacctataattactggggacataaaggaagtcgtaatgaataccagattgggtacagcaacttctggaaacaactcggctatcaaattggattgtctcagtcaagagataatgagcaacaacgccgtgatgacagattttatattaattttactctccctctgggaggaagtgttcaaagcccggtgttttccactgttttaaattatagcaaagaagagaaaaatagtattcagacatcaattagtggtactggcggggaggataatcagttctcttatggtatttcaggaaacagccaggaaaacgggccttccggttatgcaatgaatgggggttatcgttcaccttatgtaaatataaccacaacagtcgggcatgatactcagaataataatcaaaggtcatttggtgcgtcgggagcggtggtcgcacacccctatggagtgacattgagtaatgacctgagtgatacttttgccattatccatgctgaaggagctcagggggctgtcatcaataatgcctctggtagtcgtctggatttttggggaaatggtgttgttccttatgttacaccctatgagaaaaatcaaattagcatcgatccctccaatttagatttgaatgttgaattatcggcgacggagcaggaaatcattcctcgtgctaatagcgccacgttagtgaaatttgacactaaaacaggaagaagtctgttatttgatattcgtatgtctactggcaatccccctccaatggcttctgaagttctggatgaacatggacagttggccggatatgtcgctcaggccgggaaggtatttaccaggggactccctgaaaaaggtcatctcagcgttgtatggggaccagataataaagacagatgttcatttgtatatcatgttgcacacaataaagatgatatgcaatctcagctcgttcctgttctgtgtatacagcaccctaatcaggaaaaaaca

SEQ ID NO: 124 - DNA, 831 bp, Escherichia coli
atggtaaaatgtcatactctgattaaccgtagaaataaatgtctgctgattgtttttatagtccttattggatggattatattcagacctaaagcatatacttattcactaaatgataaagaaaaagagatgctcataatgttatcacaacatcctgaaactcggtactttggattttattccatagaacttccggctgattacaaaccaacaggaatggttatgttcatacaaggatcggcgatgatccctgtagaaacaaagctacaatattatcctccttttctgcaatatatgacacgatatgaggcagaactaaaaaacacctcagcattagatccactggatacgccttatttgaagcaagttcacccactaagtccacctatgaatggagtcatttttgaacgaatgaaagcgaaatacaccccagattttgcacgagtattggatgcatggaaatgggaaaatggcgttacgttttcagtaaaaatagaagctaaagatggtagagcaacccgctatgatggaattagtaagattgccgaatacagttatggatataatattccagaaaaaaaagtacagttacttactattctttcaggactacaacctcgtgcagataaccaacccccatcagaaaataaattggcgatacaatatgcacaggttgacgcttcactacttggagagtatgaattatctgtagattataaaaatagcaataatattaaaataagtttgcagacggataataatagttatattgactcattattagatataagatatccgagtaatggaaacagagcatggtataactctata

SEQ ID NO: 125 - DNA, 1098 bp, Escherichia coli
atgctacctgagcctgtttatcgacgctggattatattattaatatctatgttaacagttggtactctgtttattttatcggtctggaattctgcgacatactgggatatttttatttatggcgttctgccaatgctgtttctttggctatgtttgtttggtattgcgctgaacaaatatgaacaatccgttgcagcctgtataagttgggagtctgaaagacaacaagttaaacaactctggcaacactggagccaaaaacaactggcaatagttgggaatgttctttttacaccggaagaaaaaggcatgagtgttttactggggccacaggaagagatccctgcatatcctaaaaaggcacgaccgttattctctgcatcccgttattctctttcgtctatattccatgatattcaccagcaactgacacaacaatttcctgattatcgtcattatctacatactatctacgtattacagcctgagaaatggcgtggagaaaccgtgagacaggctattttccatcaatgggacttagtacctgaacggaccaatactcttaatcaaatccagtctctttatgatgaaagatttgacggtctaattctggttgtttgtttacaaaactggccggagaataaacctgaagatacgagtgaactggtatcagcacagcttatctcctcatcgtcatttgtacggcagcaccagatacccgttattgctggtctggggcgtgtaatgccattagaacccgaggagttggagcataatctggatgtgttatttgaatataaccaattggataacaaacaactacagcatgtctgggtctctggtttagatgagggaacgatagaaaaccttatgcagtatgctgaacaacatcaatggtcacttcctaaaaaacggcccctacacatgattgatcattcctttggccctacaggagagtttatttttcctgtctctctggcaatgctgtcagaggctgccaaagaaactgaacaaaatcatttaattatctatcagtcagcacagtatgctcagaaaaagagcctttgcctgattacccggaagctttatttaaggaca

SEQ ID NO: 126 - DNA, 780 bp, Escherichia coli
atgttgaacagaaaactaaatatacggctacgtcattccctgaacagtcactgcataccttccatcattatcaataacaccgtacgttcatttcagaggtcagtcatgaataccagagctctttttcccctgctgttcactgtggcatcattctccgcctccgccggcaactgggctgtcaaaaacggctggtgtcagaccatgacggaagatggtcaggcgctggtaatgctgaaaaatggcacgattggtattaccggcctgatgcagggatgcccgaatggtgtacagacgctcctgggcagccgtatcagtattaacggtaacctgatccccacatcacaaatgtgtaatcagcagacgggattcagggctgttgaggtggaaatcggacaggcgccggaaatggtcaaaaaagccgttcactccatagcagagcgtgatgtgtccgttttacaggcatttggtgtacgaatggaattcacccgcggtgatatgctgaaggtctgtccgaaatttgtcacatcacttgccggtttttccccgaaacagacgaccactattaataaagattccgtcctgcaggctgcccggcaggcatacgcccgggaatatgacgaggaaacaacagaaaccgctgattttggctcttacgaagtaaaaggcaataaggttgagtttgaagtattcaatcctgaagaccgtgcgtacgacaaagtgaccgtcacggttggtgctgacggtaatgccaccggcgccagcgttgaatttatcggaaaa

SEQ ID NO: 127 - DNA, 1155 bp, Escherichia coli
gtggtaattatcaatagcacgatactgagcggcgcaggcgctatcccttccctgacgtcgctcttacccgacatcagaaaaatgctgctggtcactgaccgtaatattgcgcagctcgacggtgtgcagcagattcgcgccttactggaaaagcactgcccgcaggttaacgttatcgataatgtgcccgcagagcccacgcatcatgatgtgcgccagctaatggatgcccctggcgatgcctcttttgatgtggtggtcgggatcggcggtggcagcgtgttggatgtggcgaagctgctatcggtgctttgccatccacaatcaccggggctggatgcgctgcttgcgggtgaaaaaccgactcagcgggtgcaatcatggttgattcctacaaccgccggaaccggctcagaagccacgccgaatgcgattctggcaatccctgagcaaagcacgaaggtgggtattatttcccaggtgctgttaccagactatgtggcgcttttcccggaactgaccaccagcatgcccgcgcatattgcggcgtccacgggcattgatgctctttgccacttactggagtgttttaccgcgaccgtggcaaatccggtcagcgataacgcggcgctgactgggttaagtaaacttttccggcacattcaacccgccgtgaacgatcctcaggatctgcgcgcaaaactggaaatgctgtgggcgtcttactatggcggcgtagcgataacccatgcgggcacgcatctcgttcatgcgctctcctacccgttaggtggcaaatatcatctgccgcatggcgtcgcgaatgccatcttgctggcgccgtgcatggcgtttgttcgcccctgggcggtcgagaaatttgcccgggtctgggattgcattcccgatgcggaaaccgccctgagcgcggaagaaaaatctcatgccctggtgacctggttacaggcattagtcaatcaactcaagctacccaacaatctcgcggctctcggcgtaccgccagaggatattgcctctctgagcgaggcggcactgaacgtgaagcgccttatgaacaatgtgccgtgccaaattgatctacaggacgtacaggccatttaccaaacactgtttccgcaacatccatttaaggag

SEQ ID NO: 128 - DNA, 315 bp, Escherichia coli
atgaatatcagaaaactgttttgtccgggaaacaccccccggattttattgtttttattcttttttgttgtttctgcaataaccacaattgcatgcggatacactgagaagaatgcaacaggaaatgtgctgcttctgtttctccttctgctccttgcacacagaaataccctcacatccattacagcgctgttatttctgttctgttgtgcactgtatgcgcctgccggtatgacgtacggtaaaatcaacaacagttttattgtcgcgttgttgcagaccacaactgatgaggcagcggagtttaccgggatg

SEQ ID NO: 129 - DNA, 441 bp, Escherichia coli
atgaatattcaggcaataaaagaaatggtaaatttaatttgtagttttttatttatattctttctgtcctcggcttttgtttcttttgggtgttatgctatttatgaattgtttttatggaatgatattattgtatatagctggggatatatattaattgtctttttacctttcacattatatgtaatgtcgtttgagattttgttttttgctattagtgggcgacgattgtctaaagtaacaatggtgcgcctttggttgataattaaaattattattgctttctctatttgcgcagtgttgattttttcttcaatttacaaaaaagaattattatctagaaattatattgcttgtagtggtatcccgtctgggtggatgccgggtctggcaacgaaatacgttaaagaaaaatcattatgcgaaaaaaatggcaataat

SEQ ID NO: 130 - DNA, 534 bp, Escherichia coli
atgtttcctattcgttttaaacgtccggcgttgctctgtatggcgatgctgacggttgttctgagtggctgcgggctgattcagaaagtggtggatgaatcgaaaagcgtggcctcagccgttttctacaaacaaatcaaaatactgcatctcgatttcttctcccgcagcgccctgaatacggatgcggaagatacgccgctttccacgatggtgcatgtctggcaactgaaaacccgcgaagattttgacaaggcggattacgacaccctgtttatgcaggaagagaagacgctggagaaggacgtactggcaaaacacaccgtctgggtaaaaccggaaggcacggcatccctgaatgtgccgctggataaagagacgcagtttgtcgccattattgggcagttttatcaccctgatgaaaaaagcgacagctggcgtctggtgatcaaaagggacgaactggaggccgacaagccgcgctcgattgaactgatgagaagcgacctgcgactgctgcctctcaaggataaa

SEQ ID NO: 131 - DNA, 627 bp, Escherichia coli
atgttcttaaaaagaaaatggtattacgcagtgacgacatctgtcgtcattactttgtgtggtggaggatattatatgtacaggcaagaatatcagatggttgtcactgtaccaactgctgacgcgaacgatcccaactggccaaataaaaggatacagtttgataccagcgaatggctacagcaacttcaatatattaaaatagatgatcattatatattgaatactcaatatactccaattgctaatttggatgactttggtattacattaaaattacagaacgcattaaatgggtcggataaaagacttcctgcactatatggccttgctgagatggatgctcagaaatttaaagacctgatgcgcggtaaaattaaatgtgaatatctgaggacgacatttgatgcggaaacattaaagcctgtcaatgattatttccttatttcttttacttataaagataagtggtatgaatttgagacagaaagaaaaatatctaaaacaagtgatgatgggtattttttgtgggcatttgataatactgtccacgaagcaggctattggcataacacagatccggctgcgtattcctatagagattaccagaatggtaaggctgtgaaa

SEQ ID NO: 132 - DNA, 1272 bp, Escherichia coli
atggatatttggcggggacattcgtttctgatgacaatttccgctaggttcagacaatacgttttctctcttatgtcaattttattgcaggaacgaaaaatgaatattttcactttatccaaagcaccgctatacctgttaatttcactatttttacccacgatggccatggctatcgatccacctgaacgcgaactttcgcgatttgccctgaaaacgaattaccttcagtcccctgatgaaggcgtctatgaactggcgtttgataatgccagtaaaaaggtgtttgcagcagtcaccgatcgtgtaaatcgtgaagccaataaaggctatctgtattcgtttaattcagattcgctgaaagtcgaaaataaatacacgatgccataccgggcattttcgctggcgataaatcaggataaacatcagctctatatcggacacacccagtcagcgtccctgcgtatcagtatgtttgacaccccaaccggcaaactggtaagaaccagcgacaggttaagttttaaagcggcaaacgctgcagattcgcgttttgagcattttcgccatatggtttacagccaggattccgataccctgtttgtgagttatagcaatatgctgaaaacggccgagggcatgaagcctctgcataagctgttaatgctcgacgggacgacgcttgccttaaaaggcgaggttaaggatgcttacaaaggtacagcgtatggtctgacgatggatgaaaaaacacagaaaatctacgttggcggaagagattacatcaacgaaattgatgcgaaaaatcagacgctgctgcgtaccatcccgttgaaagatccgagaccacaaatcacaagtgtgcagaatctggcggtggactccgcttctgaccgtgcctttgtggtggtattcgaccatgacgatcgttccggtacaaaagatggactctatatttttgacttacgcgacggtaaacagcttggctatgtgcacacaggagccggagctaacgcggtgaaatacaatccgaaatataacgaactgtatgtcaccaacttcactagcggcaccatcagcgtagtggatgccaccaaatacagcatcacccgtgaatttaacatgccggtctacccaaaccagatggtgttgtcggacgatatggataccctttacattggcatcaaagaaggctttaaccgcgattgggatcctgatgtgtttgtggaaggagctaaagaacgtattctgagcattgatttgaaaaagtcg

SEQ ID NO: 133 - protein, 163 aa, Escherichia coli
MAIPAYLWLKDDGGADIKGSVDVQGREGSIEVVALDHDVYIPTDNNTGKLTGTRTHKPFTFTKEIDASSPYLYKAVTTGQTLKTAEFKFYRINDAGQEVEYFNITLDNVKLVRVAPLMHDIKDPSREKHNHLERIEFRYEKITWTYKDGNIIHSDSWNERPSA

SEQ ID NO: 134 - protein, 550 aa, Escherichia coli
VRNTLKQAIVLWGMVLLLVLWSVFISPSGVLRWAGAAAIVLAVAALLIYRRRQAWTEMTGDAGLSSLPPETYRQPVVLVCGGLSAHLSTDSPVRQVSEGLYLHVPDEEQLVAQVERLLTLRPAWASQLAVAYTIMPGIHRDVAVLAGRLRRFAHSMATVRRRAGVNVPWLLWSGLSGSPLPERASSPWFICTGGEVQVATSTETTMPAQWIAQSGVQERSQRLCYLLKAESLMQWLNLNVLTALNGPEAKCPPLAMTVGLVPSLPAVDNNLWQLWITARTGLTPDIADTGTDDALPFPDALLRQLPRQSGFTPLRRACVTMLGVTTVAGIAALCLSATANRQLLRQVGDDLHRFYAVPVEEFITKARHLSVLKDDATMLDGYYREGEPLRLGLGLYPGERIRQPVLRAIRDWRPPEQKMEVTASLQVQTVRLDSMSLFDVGQARLKDGSTKVLVDALVNIRAKPGWLILVAGYTDATGDEKSNQQLSLRRAEAVRNWMLQTSDIPATCFAVQGLGESQPAATNDTPQGRAVNRRVEISLVPRSDACQDVK

SEQ ID NO: 135 - protein, 194 aa, Escherichia coli
MIKSTFWRALALTATLILTGCSHSQPEQEGRPQAWLQPGTLITLPAPGISPAVNSQQLLTGSFNGKTQSLLVMLNAEDQKITLAGLSSVGIRLFLVTYDAKGLRAEQSIVVPQLPPASQVLADVMLSHWPISAWQPQLPTGWTLRDNGDKRELRNASGKLVTEITYLNRQGKRVPISIEQHVFKYHITIQYLGD

SEQ ID NO: 136 - protein, 129 aa, Escherichia coli
MKRYIKWFAITIFISMLSACVRTAPVQQISTTVSVGHTQEQVKNAILKAGAQRKWIMTQVSPGVIKARYQTRNHVAEVRITYTATYYNIKYDSSLNLQASDGKIHKNYNRWVRNLDKDIQVNLSTGATL

SEQ ID NO: 137 - protein, 415 aa, Escherichia coli
MKRKHLLLLLLFSFSTNSAPLYSLIREAVMHDPIVMEARAELTSAQSRIEQASSAHWPVVTATGSKLLSQSHRYSYDYDTEDILPGIRGEVNIFASGAIEADVRRSESEAEYYHYKMEETKEETIHSFVSLYLDALREKQSIAVLEQSLSRHNAILNDLNTISIHDTGRESELVQAEARRLMVRQQINSRSRVLKTTLGKLSTWTKNPVTEADLENPFSRMTEAKLLTDFTQAPQKGNPSWLASQADVESKKAALKAQELARYPRVDLTGSVTRDDQQIGVNLSWDLFNRNASYGVTEKAAQIVAATGRLDSVARMIDETGRLSLITVRQSRGEMETLRRQEQASARVVDFYRLQFQVARKTLIELLNAENELYSVGLSRVQTEDQMLHGMLDYLYSQGMLLKWSGVNLSGEEEK

SEQ ID NO: 138 - protein, 201 aa, Escherichia coli
MKFLPLLALLISPFVSALTLDDLQQRFTEQPVIRAHFDQTRTIKDLPQPLRSQGQMLIARDQGLLWDQTSPFPMQLLLDDKRMVQVINGQPPQIITAENNPQMFQFNHLLRALFQADRKVLEQNFRVEFADKGEGRWTLRLTPTTTPLDKIFNTIDLAGKTYLESIQLNDKQGDRTDIALTQHQLTPAQLTDDEHQRFAAQ

SEQ ID NO: 139 - protein, 770 aa, Escherichia coli
MENFFMKNSKVFYRSALATAIVMALSAPAFATDSTVSTDPVTLNTEKTTLDQDVVINGDNKITAVTIETSDSDKDLNVTFGGHDITAASTVNQDFVEGVKVSGNKNVVINATDSTITAQGEGTYVRTAMVIDSTGDVVVNGGNFVAKNEKGSATGISLEATTGNNLTLNGTTINAQGNKSYSNGSTAIFAQKGNLLQGFDGDATDNITLADSNIINGGIETIVTAGNKTGIHTVNLNIKDGSVIGAANNKQTIYASASAQGAGSATQNLNLSVADSTIYSDVLALSESENSASTTTNVNMNVARSYWEGNAYTFNSGDKAGSDLDINLSDSSVWKGKVSGAGDASVSLQNGSVWNVTGSSTVDALAVKDSTVNITKATVNTGTFASQNGTLIVDASSENTLDISGKASGDLRVYSAGSLDLINEQTAFISTGKDSTLKATGTTEGGLYQYDLTQGADGNFYFVKNTHKASNASSVIQAMAAAPANVANLQADTLSARQDAVRLSENDKGGVWIQYFGGKQKHTTAGNASYDLDVNGVMLGGDTRFMTEDGSWLAGVAMSSAKGDMTTMQSKGDTEGYSFHAYLSRQYNNGIFIDTAAQFGHYSNTADVRLMNGGGTIKADFNTNGFGAMVKGGYTWKDGNGLFIQPYAKLSALTLEGVDYQLNGVDVHSDSYNSVLGEAGTRVGYDFAVGNATVKPYLNLAALNEFSDGNKVRLGDESVNASIDGAAFRVGAGVQADITKNMGAYASLDYTKGDDIENPLQGVVGINVTW

SEQ ID NO: 140 - protein, 660 aa, Escherichia coli
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVEASDSSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQW

SEQ ID NO: 141 - protein, 719 aa, Escherichia coli
MRDEMLYNIPCRIYILSTLSLCISGIVSTATATSSETKISNEETLVVTTNRSASNLWESPATIQVIDQQTLQNSTNASIADNLQDIPGVEITDNSLAGRKQIRIRGEASSRVLILIDGQEVTYQRAGDNYGVGLLIDESALERVEVVKGPYSVLYGSQAIGGIVNFITKKGGDKLASGVVKAVYNSATAGWEESIAVQGSIGGFDYRINGSYSDQGNRDTPDGRLPNTNYRNNSQGVWLGYNSGNHRFGLSLDRYRLATQTYYEDPDGSYEAFSVKIPKLEREKVGVFYDTDVDGDYLKKIHFDAYEQTIQRQFANEVKTTQPVPSPMIQALTVHNKTDTHDKQYTQAVTLQSHFSLPANNELVTGAQYKQDRVSQRSGGMTSSKSLTGFINKETRTRSYYESEQSTVSLFAQNDWRFADHWTWTMGVRQYWLSSKLTRGDGVSYTAGIISDTSLARESASDHEMVTSTSLRYSGFDNLELRAAFAQGYVFPTLSQLFMQTSAGGSVTYGNPDLKAEHSNNFELGARYNGNTWLIDSAVYYSEAKDYIASLICDGSIVCNGNTNSSRSSYYYYDNIDRAKTWGLEISAEYNGWVFSPYISGNLIRRQYETSTLKTTNTGEPAINGRIGLKHTLVMGQANIISDVFIRAASSAKDDSNGTETNVPGWATLNFAVNTEFGNEDQSRINLALNNLTDKRYRTAHETIPAAGFNAAIGFVWNF

SEQ ID NO: 142 - protein, 199 aa, Escherichia coli
MRKVCAVILSAAICLSVSGAPAWASEHQSTLSAGYLHARTNAPGSDNLNGINVKYRYEFTDALGLITSFSYANAEDEQKTHYSDTRWHEDSVRNRWFSVMAGPSVRVNEWFSAYSMAGVAYSRVSTFSGDYLRVTDNKGKTHDVLTGSDDGRHSNTSLAWGAGVQFNPTESVTIDLAYEGSGSGDWRTDAFIVGIGYRF

SEQ ID NO: 143 - protein, 456 aa, Escherichia coli
MKKSTLSLAIGLLLACSTGMAKTQHLTLEQRLEAAEMRAAKAEGQVKQLQTQQAAEIREIKTAQGNTPVNGQSTTESEKKNATPPNLLLSGYGDLKIYGDVEFNMDAESNHGLLAMTNADVNSDPTNEWNLNGRILLGFDGMRKLDNGYFAGFSAQPLGDMHGSVNIDDAVFFFGKENDWKVKVGRFEAYDMFPLNQDTFVEHSGNTANDLYDDGSGYIYMMKEGRGRSNAGGNFLVSKQLDNWYFELNTLLEDGTSLYNDGNYHGRDMEQQKNVAYLRPVIAWSPTEEFTVSAAMEANVVNNAYGYTDSKGNFVDQSDRTGYGMSMTWNGLKTDPENGIVVNLNTAYLDANNEKDFTAGINALWKRFELGYIYAHNKIDEFSGVVCDNDCWIDDEGTYNIHTIHASYQFANVMDMENFNIYLGTYYSILDSDGDKIHGDDSDDRYGARVRFKYFF

SEQ ID NO: 144 - protein, 174 aa, Escherichia coli
MNGKAFLACVLMSVVLTGCETAKKISQVIRNPDIQVGKLMDQSTELTVTLLTEPDSNLTADGEAAPVDVQLVYLSDDSKFHAADYDQVATTALPDVLGKNYIDHQDFNLLPDTVKTLPPIKLDEKTGYIGVIAYFSDDQATEWKQIESVESIGHHYRLLVHIRASAIEMKKEEN

SEQ ID NO: 145 - protein, 1144 aa, Escherichia coli
LTLAWIFLLVWIWWQGPKWTLYEQHWLAPLANRWLATAVWGLIALVWLTWRVMKRLQKLEKQQKQQREEEKDPLTVELHRQQQYLDHWLLRLRRHLDNRRYLWQLPWYMVIGPAGSGKSTLLREGFPSDIVYTPESIRGVEYHPLITPRVGNQAVIFDVDGVLTTPGGDDLLRRRLREHWLGWLMQTRARQPLNGLILTLDLPDLLTADKSRRETLVQNLRQQLQEIRQSLHCRLPVYVVLTRLDLLNGFAALFHSLDKKDRDAILGVTFTRRAHESDGWRSELGAFWQTWVQQVNLALSDLVLAQTGAAPRSAVFSFSRQMQGTGEIVTALLAALLDGENMDVMLRGVWLTSSLQRGQVDDIFTQSAARQYGLGNSSLATWPLVETTPYFTRRLFPEVLLAEPNLAGENSVWLNSSRRRLTAFSTCGAALAALMVGSWHHYYNQNWQSGVNVLAQAKAFMDVPPPQGTDEFGNLQLPLLNPVRDATLAYGDYRDHGFLADMGLYQGARVGPYVEQTYIQLLEQRYLPSLMNGLIRDLNIAPPESEEKLAVLRVVRMMEDKSGRNNEAVKQYMARRWSNEFHGQRDIQAQLMVHLDYALEHTDWHAQRQSSDSDAVSRWTPYDKPIINAQQELSKLPIYQRVYQTLRTKALSVLPADLNLRDQVGPTFDNVFVAGNDEKLVIPQFLTRYGLQSYFVKQREGLVELTALDSWVLNLTQSVAYSEADREEIQRHITEQYISDYTATWRAGMDNLNVRDYEAMSALTDALEQIISGDQPFQRALTALRDNTHALTLSGKLDDKAREAAINEMDYRLLSRLGHEFAPENSALEEQKDKASTLQAVYQQLTELHRYLLAIQNSPVPGKSALKAVQLRLDQNSSDPIFATRQMAKTLPAPLNRWVGKLADQAWHVVMVEAVRYMEVDWRDNVVKPFNEQLADNYPFNPRATQDASLDSFERFFKPDGILDNFYKNNLRLFLENDLTFGDDGRVLIREDIRQQLDTAQKIRDIFFSQQNGLGAQFAVETVSLSGNKRRSVLNLDGQLVDYSQGRNYTAHLVWPNNMREGNESKLTLIGTSGRAPRSIAFSGPWAQFRLFGAGQLTNVTSDTFNVRFNVDGGAMVYQVHVDTEDNPFTGGLFSLFRLPDTLY

SEQ ID NO: 146 - DNA, 489 bp, Escherichia coli
atggctattcctgcttatctctggctgaaagatgacggcggcgcggatatcaaaggttccgtggacgttcaggggcgcgaaggtagcatcgaagtggtggcgctggatcacgatgtgtacatcccgaccgacaataacaccggcaaactgaccggtacccgtactcacaagccttttacgtttaccaaagaaatcgatgcgtccagcccgtatctctacaaagctgtgaccaccggacagaccctgaaaacggcagaatttaagttttaccgcatcaacgatgccggtcaggaagtggagtacttcaacatcacgcttgataacgtcaagctggtcagagtcgctccgcttatgcacgacatcaaggatccttccagagagaagcataaccacctggaacgtattgagttccgctacgagaaaatcacctggacttacaaagacggcaacatcattcattccgactcgtggaatgagcgtccttccgcc

SEQ ID NO: 147 - DNA, 1650 bp, Escherichia coli
gtgaggaacacgctgaaacaggccatcgtgctgtggggaatggtgttactgctggtgctgtggtcagtgtttatcagtccgtctggcgtgctgagatgggccggtgcggcggctatcgttctggcggttgccgcgttgttgatttatcggcgcaggcaggcgtggacggagatgaccggcgatgccgggttgtcatcgctgccgccggaaacctaccgacagccggtagtgctggtctgtggcggtctgtcggcgcacctgtccactgacagcccggtccgccaggtttcagaagggctgtatctgcatgttcctgatgaagaacagcttgtggcgcaggtggagcgattgctgacccttcgcccggcgtgggcatcgcagcttgccgtggcgtataccatcatgcccggcatacaccgggatgtggcggttctggccggacggctgcgacggttcgcccacagtatggcgacggtgcgtcgtcgggcaggcgtaaacgtcccctggcttctctggagcgggctgtccggctcgccgttgccggaaagagcgagttcaccgtggtttatctgtaccggcggcgaagttcaggtagcaacatccacagagaccaccatgcccgcgcagtggattgcacaatccggcgtacaggagcgcagtcagcgactctgttacctgctgaaagctgaaagcctgatgcagtggctgaatcttaatgtgctgacggcactgaacggcccggaggcgaaatgtccaccactggcgatgaccgtggggctggtcccctcgttgcctgcggtggataacaacctgtggcagttgtggatcaccgccagaaccggcctgacgccggatatcgcggacaccggcacagacgatgcgctgccattcccggatgccctgttacggcagttgccgcgtcagtcgggctttaccccgctgcgacgagcctgcgtgaccatgctgggcgtcaccaccgtggcgggtatcgccgcgctgtgcctgtcagccacggcaaatcgccagttattacggcaggtcggtgacgatctgcaccggttttatgccgtcccggtggaggaatttatcaccaaagcccgtcacctgtcggtgctgaaagacgatgcgaccatgctcgatgggtattaccgggaaggagaacccctgcgcctcggtctggggttataccccggcgaacgcatccgccagccggtattacgcgccattcgcgactggcgtccgcctgaacaaaaaatggaggtgacggcttcgcttcaggttcagaccgtgcgtcttgacagtatgtcgctgtttgacgtcggacaggcccgcctgaaagacggctcgacaaaagtgctggtggacgcactggtgaacatccgggcaaaaccgggctggctgatcctcgtggccggatataccgatgccaccggcgatgaaaaaagcaatcagcagttatcgctgcggcgtgccgaagcggtgcgcaactggatgctgcagaccagcgacatcccggccacctgttttgccgtacagggactgggcgagagccagcctgcggcgaccaacgacacgccacagggccgggcagtcaaccggcgtgtcgaaatcagtcttgttccgcgttctgacgcctgtcaggacgtgaaa

SEQ ID NO: 148 - DNA, 582 bp, Escherichia coli
atgatcaaatccacattctggcgagcgctcgccctgaccgctacgcttatcctcactggctgtagccactcgcaaccggaacaggaaggccgcccgcaggcgtggctgcaacctggtacgctcatcacgctgcctgcgccggggatttcacccgcagtcaattcccagcaactgttgaccggcagcttcaacggcaaaacccagtctctgctagtgatgcttaatgccgaagatcagaaaatcacccttgccgggctgtcgtcggtcggcattcgcctgtttctggtgacctacgatgcaaaagggctacgcgccgagcaatccatcgtcgtcccacagttaccgcccgcaagtcaggtactggctgacgtgatgctcagccactggccgattagcgcctggcaaccgcaacttcccacaggctggacgcttcgcgacaacggcgacaaacgcgagctgcgtaacgccagcggcaaactggtcacggaaatcacctatctgaatcgccagggaaaacgcgtgccaatcagcattgagcagcatgtctttaaataccacatcaccattcaatacttaggtgac

SEQ ID NO: 149 - DNA, 387 bp, Escherichia coli
atgaaacgttatataaaatggtttgccatcacaatttttatcagtatgttgagtgcctgtgtccgtacggccccagtgcaacagataagcaccactgtcagtgtgggtcatactcaggagcaggttaaaaatgccattttgaaagcaggtgcgcagcgcaagtggattatgacgcaagtgtcccctggagttattaaagctcgctatcaaacacgaaatcacgttgcagaggttcgtattacatatacagctacctactataacatcaaatatgacagtagcctgaatctgcaggcttctgatggaaaaattcataaaaactataaccgctgggtgcgtaacctggataaagatatacaggttaacttatctacaggagcaacgtta

SEQ ID NO: 150 - DNA, 1245 bp, Escherichia coli
atgaagcgtaaacatttgttattattattgttgttttcattttccactaacagtgcgcctctttactccttaattagggaggcagttatgcacgatcccatagtaatggaagcccgggcggagttaacttcggcacaatcccgcatagagcaggcaagctctgcacattggccagttgtcacagctacaggaagtaaactcctttcacaaagtcaccgttattcctacgattatgacactgaagatattttacccggtattcgtggtgaagtgaatatatttgcttcaggggctattgaggcggatgtgcgtcggagtgagtcagaagccgaatattatcattataaaatggaagaaacaaaagaggaaacaattcactcttttgtttcattatatcttgatgcactcagggaaaaacaatccattgcggtacttgaacagagcctttcccggcataacgcaattcttaatgacctgaataccatcagtattcatgataccgggcgggagtctgagcttgttcaggccgaagccagaaggttgatggttcggcagcagataaattctaggagcagagtacttaaaaccacgctgggaaaactgtccacttggacaaaaaatccggtaaccgaagctgatcttgaaaatcctttttctaggatgacagaggccaaattattaactgattttacacaggctccacagaaaggtaacccgtcgtggcttgccagccaagctgatgttgagagtaaaaaagcggcactgaaagcacaggagcttgcccggtaccctcgggtggatttaacggggtctgtaacccgggatgaccagcagataggggtcaatctgtcttgggacctctttaaccgtaatgccagttatggtgttacagaaaaagctgcgcaaatagtggcagctaccggacgactggactctgtcgcccgaatgattgatgaaaccgggcgattatctctgataacagtcagacaaagtcgaggggaaatggaaacgctcagacgtcaggaacaggcttcagccagagttgtggacttttatcgtcttcagtttcaggtggcaagaaaaacactgattgaattactgaatgctgaaaacgaactgtacagtgtcggactctcccgggttcagacggaggatcagatgctccacggtatgctggattatctgtattcccagggaatgctcctgaaatggagcggagtgaatctttctggtgaagaagaaaaa

SEQ ID NO: 151 - DNA, 603 bp, Escherichia coli
atgaaatttttaccgctgctggcgctgctgattagcccgtttgtgagcgccctgaccctggacgatcttcagcaacgctttaccgaacaaccggtgatccgcgcccattttgatcaaacccggacgattaaagatctgccgcagccgctgcgatctcagggtcagatgttgatcgcccgcgaccaggggttattgtgggatcaaacctcaccgttccccatgcagctattgctggatgataaacgcatggtgcaggtgatcaacggtcagccgccgcaaatcatcacggcagaaaacaacccgcagatgttccagtttaaccacctgctgcgcgcgctgttccaggccgatcgcaaagtgctggaacaaaacttccgcgtcgaatttgctgacaaaggcgaaggccgctggacgctgcgcctgacgccgaccaccacgccgctggataaaattttcaacaccatcgatctcgccgggaaaacctatctggagagcattcaacttaatgataaacagggcgatcgcaccgatattgctcttacccaacatcaactgacgccagcgcaactgaccgatgacgaacaccaacgttttgccgcccag

SEQ ID NO: 152 - DNA, 2295 bp, Escherichia coli
atgaaaaacagtaaggtattttaccgcagcgcattagcgacagctattgttatggctctttctgcaccagcattcgctactgatagcacggtatcaactgatccggttacgctgaatacagagaagacgactctggatcaagatgttgttattaacggtgataacaagattacagccgtaacaattgaaacgtcagattcagataaagaccttaatgttacttttggcggtcacgatattaccgccgcatcaacggtaaaccaagatttcgttgaaggtgtaaaagttagtggtaacaaaaatgttgtgattaatgctacagactccaccatcacagctcaaggtgaaggcacctatgtccggactgcaatggtcattgattcaactggcgatgttgttgttaatggcggtaatttcgttgcaaaaaatgaaaaaggtagtgcgacagggatatctctggaagcgaccacgggaaataatttaacgctcaatggtacaaccataaatgctcaaggtaataagagttacagcaacggctctacggcaatttttgctcaaaagggtaatttgttgcagggttttgacggtgatgcaaccgacaacatcacccttgctgactcaaatattattaatggcgggattgaaacaatagttactgccgggaataagacgggaattcatacagtcaacctgaatattaaggatggctcagtaattggggcggctaataataaacaaacaatttatgcctctgcttcggcacaaggcgcaggttcagcaacgcaaaatttaaatttgtctgttgctgattcaaccatctactctgatgtcctggccctttctgaaagcgagaattcagccagtaccacaacaaatgtaaatatgaacgttgcccgctcttactgggaaggtaatgcttataccttcaatagcggcgataaagcgggtagtgatctggatataaatctttccgatagttcagtctggaaaggcaaagtttcaggggcaggagatgccagtgtatctctgcaaaacgggtctgtctggaatgttacgggttcctcaactgttgatgctctggcagtaaaagacagtacggttaatatcacgaaggctacagtcaatactggcacgtttgcttctcaaaacggcactctgattgttgatgcctcttctgaaaacactctggatatcagcggtaaagcgagcggtgacttgcgtgtttacagtgcgggttcattggatcttatcaatgaacaaacggcatttatttctaccggcaaagacagcactctaaaagccacaggcacaacggaaggtggtctgtatcaatatgacctgacacagggagctgatggtaacttttatttcgtaaaaaacacgcataaagcatccaacgccagctccgtgattcaggcaatggcagctgctccggctaacgtcgctaatctgcaggctgacacgctctccgcccgtcaggatgctgtccgtctgagcgaaaatgacaagggtggcgtatggattcagtactttggcggtaaacagaaacataccaccgcgggaaatgcatcctatgacctggatgtaaatggtgtaatgctgggtggtgatacccgcttcatgactgaagatggtagctggctggccggtgtggcgatgtcttctgcgaaaggtgacatgactaccatgcagagcaaaggtgacactgaaggttacagcttccacgcttacctgagccgccagtataacaacggtatcttcattgatactgctgcacagtttggtcactacagcaacacggcagatgttcgcctgatgaatggtggcggtaccatcaaagctgactttaacaccaatggttttggtgcgatggttaaaggcggttacacatggaaagacggtaatggcctgtttattcagccatatgccaaactgtctgctctgactctggaaggtgtggattatcaactcaacggcgtggacgttcattctgacagctataactctgtgctgggtgaggccggtacgcgcgtgggttatgacttcgctgtgggcaacgcgaccgttaaaccttatctgaatctggccgcactgaacgaattctctgatggcaacaaagtccgtctgggtgatgagtctgtcaatgccagcattgacggtgcagcattccgcgtgggtgcaggtgtacaagctgatatcaccaaaaacatgggagcatatgcaagccttgactacaccaaaggtgacgacattgagaacccgctacagggtgtagttggtatcaatgtgacctgg

SEQ ID NO: 153 - DNA, 1980 bp, Escherichia coli
atgtcacgtccgcaatttacctcgttgcgtttgagtttgttggctttggctgtttctgccaccttgccaacgtttgcttttgctactgaaaccatgaccgttacggcaacggggaatgcacgtagttccttcgaagcgcctatgatggtcagcgttatcgacacttccgctcctgaaaatcaaactgctacttcagccactgatttgctgcgtcatgttcctggaattactcttgatggtaccggacgaaccaacggtcaggatgtaaatatgcgtggctatgatcatcgcggcgtgctggttcttgtcgatggtgttcgccagggaacggataccggacacctgaatggcacttttctcgatccggcgctgatcaagcgtgttgagattgttcgcggaccttcagcattactgtatggcagtggcgcgctgggtggagtgatctcctacgatacggtcgatgcaaaagatttattgcaggaaggacaaagcagtggttttcgtgtctttggtactggcggcacgggggaccatagcctgggattaggcgcgagcgcgtttgggcgaactgaaaatctggatggtattgtggcctggtccagtcgcgatcggggtgatttacgccagagcaatggtgaaaccgcgccgaatgacgagtccattaataacatgctggcgaaagggacctggcaaattgattcagcccagtctctgagcggtttagtgcgttactacaacaacgacgcgcgtgaaccaaaaaatccgcagaccgttgaagcttctgatagcagcaacccgatggtcgatcgttcaacaattcaacgcgatgcgcagctttcttataaactcgccccgcagggtaacgactggttaaatgcagatgcaaaaatttactggtcggaagtccgtattaatgcgcaaaacacggggagttcaggcgagtatcgtgaacagataacaaaaggagcaaggctggagaaccgttccactctatttgccgacagtttcgcttctcacttactgacatatggcggtgagtattatcgtcaggaacaacatccgggtggcgcgacgacgggcttcccgcaagcaaaaatcgattttagctctggttggctacaagatgagatcaccttacgcgatctgccgattaccctgcttggcggaacccgctatgacagttatcgcggtagcagcgacggctacaaagatgttgatgccgacaaatggtcatctcgtgcggggatgactatcaacccgaccaactggctgatgttatttggctcatatgctcaggcattccgcgccccgacgatgggcgaaatgtataacgattctaaacacttctcgattggtcgcttctataccaactattgggtgccaaacccgaacttacgtccggaaactaacgaaactcaggagtacggttttgggctgcgttttgatgacctgatgttgtccaatgatgctctggaatttaaagccagctactttgataccaaagcgaaagattatatctccacgaccgtcgatttcgcggcggcgacaactatgtcgtataacgtcccgaacgccaaaatctggggctgggatgtgatgacgaaatataccactgatctgtttagccttgatgtggcctataaccgtacccgcggcaaagacaccgataccggggaatatatctccagcattaacccggataccgttaccagtaccctgaatattccgatcgctcacagcggcttctctgttggttgggtcggtacgtttgccgatcgctcaacacatatcagcagcagctacagcaaacaacctggctatggtgtgaatgatttctacgtcagttatcaagggcagcaggcgctcaaaggcatgaccactactctggtattgggcaacgccttcgataaagagtactggtcgccgcaaggcatcccacaggatggtcgtaacggaaaaattttcgtgagttatcaatgg

SEQ ID NO: 154 - DNA, 2157 bp, Escherichia coli
atgagggatgaaatgttatataatataccttgtcgaatttatatcctttccactctgtcattatgcatttctgggatagtttctactgcaaccgcaacttcttcagaaacaaaaatcagcaacgaagagacgctcgtcgtgaccacgaatcgttcggcaagcaacctttgggaaagcccggcgactatacaggttattgaccaacaaacattgcagaactccaccaatgcctccatagccgataatttgcaggacatccccggagtagagataacagacaactccttggcaggccgtaaacaaatccgcattcgtggcgaagcatcctcccgtgttttaattctcattgatggtcaggaggtaacttatcagcgcgccggagataattatggtgtgggactgttgatagatgagtctgcgctggagcgtgttgaggtagtgaaaggtccatattccgtactgtacggttcacaggcaattggcggtattgttaacttcataaccaaaaagggaggtgacaaacttgcatctggagttgtgaaagctgtttataattccgcaacagcaggctgggaagaatcaatcgcggtccaggggagcatcggtggatttgattatcgcatcaacggtagttattctgatcagggcaatcgtgatacgccggatggacgtctgccgaataccaactatcgtaacaatagtcagggtgtatggttgggttataactccggaaaccatcgttttggcctctcgcttgatcgctacagactcgcgacgcaaacttactatgaggatccagacggaagctatgaggcatttagtgtcaaaatacctaaacttgaacgagagaaagttggggtattctatgacacagacgtggacggtgactatctaaaaaaaattcatttcgacgcgtatgagcagaccatccagcgccaatttgccaacgaagtaaaaacgacacagcctgttcccagtccgatgattcaggctctgaccgttcataacaagactgacacccatgataagcaatacactcaggcggtcacattgcagagtcacttttcgctgcctgctaataatgaacttgttaccggtgcacagtacaaacaagacagggtcagccaaaggtccggtggcatgacctcaagcaaatctctgaccggcttcattaataaggaaacacgaactcgctcctattatgagtcagagcaaagtacagtctcactattcgcacaaaatgactggcgattcgccgatcactggacatggacaatgggagttcgccaatactggctttcttcaaagttgacgcgtggtgacggagtatcatataccgcaggcattataagcgatacctctcttgccagagagtctgcgagtgatcacgaaatggtaacatctacaagcctgcgctattcaggtttcgataacttggagttacgcgctgcgttcgcgcaaggctacgtatttcccacactctcccagctttttatgcagacatctgcgggcggcagtgtcacatacggaaatcctgatcttaaggctgaacactccaataactttgaattaggtgcacgatataatggtaatacgtggctgattgacagcgcagtttactactcagaagctaaagattatattgcaagtctgatctgtgatggcagtatagtttgcaatggtaacaccaactcctcccgtagtagctactattattatgacaatattgatcgggcaaaaacatggggactggaaataagcgcggaatataatggctgggttttctcgccatatatcagtggcaatttaattcgtcggcaatatgaaacttcaacattaaaaacaactaatacaggagaaccagcgataaacggacgtatagggctgaaacatactcttgtgatgggtcaggccaacataatctctgatgtttttattcgtgctgcctctagtgcaaaagatgacagtaacggtaccgaaacaaatgttccgggctgggccactctcaactttgcagtaaatacagaattcggtaacgaggatcagtcccggattaacctagcactcaataacctgacagacaaacgctaccgtacagcacatgaaactattcctgcagcaggttttaatgcagctataggttttgtatggaatttc

SEQ ID NO: 155 - DNA, 600 bp, Escherichia coli
atgcgtaaagtttgtgcagtcattttgtccgcagccatctgtctgtccgtatccggtgcgcctgcatgggcgtctgaacatcagtccacactgagcgcggggtatcttcatgcccgtacgaacgctcccggcagcgataatctgaacgggattaacgtgaaataccgttatgagtttacggacgcgctggggctgattacgtccttcagttatgccaatgctgaggatgagcaaaaaacgcactacagcgatacccgctggcatgaagattccgtgcgtaaccgctggttcagcgtgatggcggggccgtctgtacgcgtgaatgaatggttcagcgcgtattcgatggcgggtgtggcttacagccgtgtgtcgactttctccggggattatctccgcgtaactgacaacaaggggaaaacgcacgatgtgctgaccggaagtgatgacggtcgccacagcaacacgtctctggcgtggggggctggcgtgcagtttaacccgaccgaatccgtgaccattgaccttgcttatgaaggttccggtagtggcgactggcgaacggatgcatttattgttggtatcggataccgtttctga

SEQ ID NO: 156 - DNA, 1368 bp, Escherichia coli
atgaaaaaatcgacattatctttagccatcggtttattattggcatgtagtaccggtatggcaaaaacacagcatttaacgctggaacaacgcctggaagcggcagaaatgcgggcagcaaaagcagaggggcaggttaaacagcttcagacacaacaagccgccgagatccgcgaaattaaaaccgcacagggcaacacgccggtaaacggtcaatcaacgacggagtcagagaagaaaaacgccaccccgcctaatctcctgctttcagggtatggcgatttaaaaatctacggtgacgtagaatttaatatggatgcggaaagtaatcatggcctgctggcaatgaccaacgctgatgtgaatagcgatcccactaatgaatggaatctcaatggtcgtattctgttaggttttgatggtatgcgaaaactggataatggctatttcgctgggttctccgcacaaccgctgggggatatgcacggttcagtaaatatcgatgatgcggttttcttctttggcaaagaaaacgactggaaggtcaaagtaggccgttttgaagcctacgatatgttcccgctgaatcaggatacctttgttgaacattccggtaatactgcgaacgatctttatgacgatggcagcggttatatctatatgatgaaagagggccgcggacgttctaacgctggcggtaatttcctcgtcagcaaacaactcgataactggtattttgaattaaacacgttactggaagacggaacatctttatataacgacggtaattatcatggacgcgatatggaacagcagaaaaatgttgcttatctgcgtccggtaattgcctggtcgccgacggaagaattcaccgtttccgcagcgatggaagcgaatgtggtaaataatgcttatggttataccgatagcaagggtaattttgtcgatcagtccgatcgtaccggttatggcatgagtatgacctggaatggcctgaaaaccgatccggaaaatggcatcgtggttaatcttaataccgcctatttagatgctaataatgaaaaagatttcacggcagggattaacgcgctgtggaaacgtttcgagctgggttatatctatgcacataataagattgatgaatttagtggcgtggtttgtgataacgattgctggattgatgatgaaggaacatacaacattcacaccattcatgcgtcttatcagttcgctaatgtgatggatatggagaactttaatatttacctcggcacgtattactccattctggatagcgacggcgataagatacacggcgacgatagtgatgaccgttacggcgcacgcgttcgctttaaatacttcttc

SEQ ID NO: 157 - DNA, 522 bp, Escherichia coli
atgaacggcaaagcgtttctggcctgcgttctgatgagcgtcgtattaactggctgtgaaacagcgaaaaaaatcagccaggtgatccgcaatccggatattcaggtcggaaagctgatggatcagtcaaccgagctgaccgtcacgctgctgaccgagccggacagcaacctgacggcggatggcgaagccgcgccggtggatgtccagttggtttatctgagcgacgactcaaaattccatgccgccgactacgaccaggttgccaccaccgcgctgcccgacgtgctggggaaaaactatatcgatcaccaggacttcaacctgttgccggataccgtaaaaacactgccgccgatcaagttggatgagaaaaccggttatatcggtgtcattgcctatttttcagacgaccaggccacagaatggaaacaaattgagtcggtagaaagtatcggccaccactatcgcctgctggtgcatatccgcgccagtgcgattgagatgaaaaaagaggaaaac

SEQ ID NO: 158 - DNA, 3432 bp, Escherichia coli
ctgacgctggcatggatttttctgctggtgtggatctggtggcagggtccaaaatggacgctctatgagcagcactggctggctccgctggcaaaccgctggctggcgaccgccgtctggggacttatcgctctggtctggctcacctggcgggtgatgaagcgtctgcaaaagctggaaaaacagcagaaacagcagcgggaggaagaaaaagatccgttgaccgtggaactccaccgccagcagcaatatctggatcactggctgctgcgcctgcgccgccatctggataaccgccgttatctgtggcagttgccgtggtatatggtcattggtcctgcgggtagcggcaaaagcacgctgctgcgcgagggctttccgtctgacattgtttacacgccggaaagcatccggggtgtggaataccacccgctgatcacaccgcgagtgggcaaccaggcggtaattttcgatgttgacggcgtactgaccactcccggcggggatgatctgctccgccgccgcctgcgcgaacactggctgggctggctgatgcaaacgcgcgctcgccagccgctcaacggtcttatcctgacgctcgatcttcccgatctgctgacggcggataaatcccgccgtgagacactggtacaaaatttgcgccagcaacttcaggagatccgtcagagcctgcactgccgtctgcccgtttacgtggtgctgacacggctggatctgctgaacggctttgccgcgctgttccattcactggataaaaaagaccgcgatgcgatcctcggcgtcacatttacccgccgcgcccatgaaagtgacggctggcgcagcgaactgggggctttctggcagacgtgggtacaacaggtgaacctggcgctgtcggatctggtgctcgcacaaaccggtgctgctccccgcagcgctgtgttcagcttctcccgtcagatgcagggaacaggagaaatcgtcaccgcactgctcgccgcattgctggacggtgagaacatggatgtaatgctgcgtggcgtctggctcacatcctcgctacagcgtggccaggtggatgatattttcacgcagtccgccgcccgccagtacggactgggtaacagctcgctggcaacctggcctctggtggagacgacgccgtattttactcgccgcctcttcccggaagtcctgctggctgagccgaacctggcgggtgaaaacagcgtctggctgaacagctcccggcgcaggctgaccgccttttccacctgtggcgcggcactggcggcattgatggtcggaagctggcaccattattacaatcagaactggcagtctggcgttaacgtactggcacaagctaaagcctttatggacgtaccaccaccgcagggaacggatgaattcggcaatctgcaattgccattgcttaacccggtacgcgatgccaccctggcctatggtgattatcgcgatcacggttttctggcggatatgggattgtaccagggcgcccgcgtagggccgtatgtggagcaaacctacattcagcttcttgagcagcgttatctcccctcgttaatgaacggcctgatccgggatctaaacattgccccgccagagagcgaagaaaagctcgctgtgctgcgcgtagtgcgcatgatggaagacaaaagtgggcgcaacaacgaggcggtaaaacagtacatggcacggcgctggagcaatgaatttcacggccagcgcgatattcaggcgcaactgatggtgcatctggactatgcgctggagcacaccgactggcacgcgcagcgccaaagcagcgacagcgatgctgtcagccgctggaccccctatgataaaccgatcattaatgcgcagcaggaactgagcaagctgcccatataccagcgtgtctaccagaccctgcgcaccaaagcattaagcgtgttgcccgccgatttgaatttgcgcgaccaggttggtcccaccttcgacaacgtgttcgtcgccggtaatgatgaaaaactggtgatcccgcagttcctcacccgctatggactgcaaagctattttgtcaaacagcgtgagggcctcgttgagctgaccgcgctggattcgtgggtactgaacctgacgcaaagcgtcgcctacagcgaggccgaccgtgaagagatccagcgccatatcaccgaacagtacatcagtgactataccgccacctggcgtgccggaatggataacctcaacgtccgtgactatgaggccatgtcggcgctgaccgacgcgctggagcagattatcagcggcgatcagccattccagcgtgcgctgacggcgctgcgcgataatacccacgcgctgacgctctccggcaaactggatgataaggcgagggaagcggcgataaatgagatggattaccgcctgttatcccggctggggcatgagttcgcaccggaaaacagcgcactggaggagcaaaaggacaaggcgagtacgctacaggccgtgtaccagcaactgaccgagctgcaccgttacctgctggcgatccagaactcgccagtgccggggaaatcggcgctgaaagcagtacagctacggctggatcaaaacagcagcgatccaatcttcgccacccgtcagatggcaaaaaccctgcctgcgcctcttaaccgctgggtaggtaagctcgcggatcaggcctggcatgtggtgatggtggaagccgttcgttacatggaagtggactggcgcgacaatgtagtgaaacccttcaacgagcagcttgccgataactatccgtttaatccgcgcgccacacaggatgcctcactggattcgtttgaacgtttctttaaaccggatggcattctggacaatttctacaagaacaacctgcgcctgttccttgaaaacgatctgacctttggcgacgacggcagagtgttaatccgtgaagatatccggcagcaactggataccgcgcagaaaatccgcgacatcttcttcagccagcagaacgggctgggcgcacagtttgccgtggaaaccgtatcgctttccggcaataagcggcgcagcgtacttaacctggacggccagttagtggactacagccagggacgcaactacaccgcccatctggtctggccgaacaacatgcgtgaaggcaatgaaagcaagctgacgctgattggcaccagcggcagagcaccgcgcagtatcgcgttcagtggaccgtgggcgcagttccgcctgttcggcgcgggccagttgaccaatgtgaccagtgacacctttaacgtgcgctttaacgtggacggcggcgcaatggtttaccaggtgcatgtggataccgaagataacccgttcaccggcggtctgttcagcctgttccgtttaccggatacgttgtat

SEQ ID NO: 159 - protein, 725 aa, Escherichia coli; identical to SEQ ID NO: 8

SEQ ID NO: 160 - DNA, 2175 bp, Escherichia coli; identical to SEQ ID NO: 74

(19) United States
(12) Patent Application Publication
(10) Pub. No.: US 2010/0316647 A1
(43) Pub. Date: Dec. 16, 2010

(54) COMPOSITIONS OF POLYPEPTIDES SPECIFIC TO PATHOGENIC STRAINS AND THEIR USE AS VACCINES AND IN IMMUNOTHERAPY

(75) Inventor: SONIA ESCAICH, PARIS (FR)

Correspondence Address: NIXON & VANDERHYE, PC, 901 NORTH GLEBE ROAD, 11TH FLOOR, ARLINGTON, VA, 22203, US

(73) Assignee: MUTABILIS, ROMAINVILLE (FR)

(21) Appl. No.: 12/840,647

(22) Filed: Jul. 21, 2010

Related U.S. Application Data
(63) Continuation of application No. 10/594,461, filed on Jan. 3, 2007, now abandoned.

(30) Foreign Application Priority Data
Mar. 26, 2004 (EP) 04 290 818.6

(51) Int. Cl. A61K 39/108; C07K 14/245; C12N 15/63; C12N 1/21; A61K 39/395; A61P 31/04

(52) U.S. Cl. 424/139.1; 530/350; 435/320.1; 435/252.33; 424/190.1

(57) ABSTRACT

The invention relates to compositions of polypeptides specific to pathogenic strains comprising at least one polypeptide of a first group, having a sequence selected in the group comprising the sequences of SEQ ID No 159, or homologous sequences of polypeptides of the first group and/or the second group with a minimum of 25% of identity with the whole sequences of said polypeptides. Application for the preparation of vaccine compositions specific to E. coli extra-intestinal infections.

This application is a continuation of application Ser. No. 10/594,461 (U.S. Patent Application Publication No. US-2009-0081224-A1), filed Jan. 3, 2007 (pending), which is a U.S. national phase of International Application No. PCT/EP2005/003972, filed 29 Mar. 2005, which designated the U.S. and claims benefit of EP 04290818.6, filed 26 Mar. 2004, the entire contents of each of which is hereby incorporated by reference in this application.

The invention relates to new compositions of polypeptides specific to pathogenic strains, particularly to extra-intestinal E. coli strains.

It more particularly relates to combinations of antigenic polypeptides and combinations of antibodies directed against said polypeptides and to their use as vaccines and in immunotherapy, respectively.

Although Escherichia coli is probably the best known bacterial species and is one of the most common isolated in clinical microbiology laboratories, misconceptions abound regarding the various types of E. coli and the infections they cause.

E. coli strains of biological significance to humans can be broadly classified in 3 major groups:

1. Commensal strains, which are part of the normal flora.

2. Intestinal pathogenic strains, which are not part of the normal flora. This group contains various pathotypes (EPEC, EHEC, ETEC, EIEC) not including Shigella.

3. Extra-intestinal strains (ExPEC) which are responsible for infections outside the gastro-intestinal (GI) tract, but can also be part of the normal flora. All hosts, either immunocompromised or not are susceptible to these infections.

ExPEC strains are responsible for the majority of the urinary tract infections (UTI) particularly cystitis, pyelonephritis, and catheter associated infections.

They are also responsible for abdominal infections, nosocomial pneumoniae, neonatal meningitidis, soft tissue infections, and bone infections. Each one of these localizations can lead to bacteremia with a risk of sepsis in case of organ failure. ExPEC strains are indeed the most common Gram negative bacilli isolated from blood cultures.

750 000 cases of bacterial sepsis occur each year in the US, and are responsible for 225 000 deaths. In a recent study on 1690 cases of sepsis, it was shown that the main bacteria species identified is ExPEC (16% of the cases) and then S. aureus (14% of the cases).

These numbers demonstrate the importance of ExPEC strains in both hospital and community acquired infections.

ExPEC strains correspond to a homogenous subset of E. coli strains. Analysis of phylogenetic relationships among E. coli strains by MLEE has revealed that E. coli belong to 4 main phylogenetic groups designated A, B1, B2 and D.

The pathogenesis of ExPEC strains is that of extra-cellular microorganisms, i.e., they are well adapted to growth in the extra-cellular fluids and efficiently resist phagocytosis by polymorphonuclear. Initial studies have shown that virulence factors known to be important for the extra-cellular growth are mainly found in B2/D E. coli., thus suggesting that B2/D subgroups contain most of the ExPEC strains. This was reinforced by experiments performed on animals showing that B2/D strains are more virulent than A and B1 strains. Subsequent epidemiological studies have indeed confirmed these hypotheses. B2/D isolates are those predominantly responsible for neonatal meningitidis (87%) and community or nosocomial acquired urosepsis, (93% and 85%, respectively).

Similar results have been reported for cystitis (70% are due to the sole B2 E. coli), thus demonstrating that the importance of ExPEC strains.

These recent findings demonstrate that the B2/D subgroup of strains is the E. coli core genome the best adapted to growth in extra-cellular fluids.

In addition to this core genome, ExPEC strains have various pathogenicity islands which encode virulence factors associated with the different pathogenesis of extra-intestinal E. coli infections (UTI, urosepsis, neonatal meningitidis . . . ). Among the main virulence factors are the capsule, which is well-known to be important for extra-cellular growth, and the iron chelation systems (aerobactin and enterochelin, for example). In addition, depending on the pathogenesis, these strains can produce toxins (CNF, hemolysin . . . ), adhesins (pap, sfa . . . ) and other iron chelation systems.

The notion that B2/D E. coli correspond to a distinct subset of pathogenic E. coli strains is reinforced by the fact that B2/D E. coli are not broadly isolated from the stools of humans. They were recovered from only 11% of individuals, whereas A and B1 subgroups are present in the stools of 74% of the individuals of a human population.

As mentioned above the pathogenesis of ExPEC strains relies on their ability to multiply in the extra-cellular fluids and to resist bactericidal activity of the complement and phagocytosis by polymorphonuclear. Therefore, as for other extra-cellular pathogens (Haemophilus influenzae, Streptococcus pneumoniae and Neisseria meningitidis) a protective antigen against ExPEC has to induce antibodies that promote opsonisation and/or the bactericidal activity of serum.

Considering the above statements, an efficient antigen has to be largely represented among the population of B2/D E. coli.

Similarly to other extra-cellular pathogens, the capsular polysaccharide would be an ideal antigen, however most pathogenic B2 strains express the K1 polysaccharide. The latter has a structure identical to that of group B meningococcus, which is non-immunogenic and shares common antigens with the brain. Another possible target may be the lipopolysaccharide (LPS). However there are a large number of different LPS serotypes that are shared by various subgroups.

The inventors have now found that some specific compositions of polypeptides coded by the B2/D genome, but absent from A and B1 E. coli strains, are particularly useful as antigens and can specifically prevent the pathologies due to ExPEC strains. Homologs of these antigenic components can be found in other pathogenic bacterial species and therefore are useful to prevent the pathologies caused by these bacteria. Accordingly, any reference to products specific to ExPEC strains and to their uses will encompass components in these species.

For example homologous antigens could be present in the following species and be as such used for prevention of disease due to the bacteria:

Pseudomonas aeruginosa, Escherichia coli O157:H7, Yersinia pestis, Vibrio cholerae, Legionella pneumophila, Salmonella enterica, Salmonella typhimurium, Haemophilus influenzae, Neisseria meningitidis, Neisseria gonorrhoeae,

Bacillus anthracis, Burkholderia cepacia, Campylobacter jejuni, Chlamydia pneumoniae, Chlamydia trachomatis, Clostridium botulinum, Clostridium difficile, Cryptococcus neoformans, Enterobacter cloacae, Enterococcus faecalis, Helicobacter pylori, Klebsiella pneumoniae, Mycobacterium leprae, Mycobacterium tuberculosis, Pseudomonas aeruginosa, Salmonella paratyphi, Salmonella typhi, Staphylococcus aureus, Klebsiella pneumoniae, Listeria monocytogenes, Moxarella catarrhalis, Shigella dysenteriae, Shigella flexneri, Shigella sonnei, Staphylococcus epidermidis, Streptococcus pneumoniae, and any species falling within the genera of any of the above species.

It is then an object of the invention to provide new combinations of isolated antigenic polypeptides, and new combinations of isolated polynucleotides belonging to the core B2/D genome and not present in commensal E. coli.

Another object of the invention is to provide new combinations of antibodies raised against the antigenic polypeptides of said combinations, or peptidic fragments thereof.

It is still another object of the invention to provide vectors and host cells containing said polynucleotides.

Another object of the invention is to provide vaccine compositions specific to extra intestinal infections caused by ExPEC and pathologies caused by other pathogenic strains expressing antigenic polypeptides homologous to the ExPEC antigenic polypeptides.

The invention also relates to means for detecting and treating a development of E. coli in a human or animal compartment which is extra-intestinal (systemic and non-diarrhoeal infections, such as septicaemia, pyelonephritis, or meningitis in the newborn).

The combinations of isolated antigenic polypeptides used according to the invention are selected among polypeptides specific to B2/D E. coli strains and not present in A and B1 isolates of E. coli. They are encoded by genes belonging to the core B2/D genome and are not present in commensal E. coli.

They comprise at least one polypeptide of a first group, having a sequence selected in the group comprising the sequences of SEQ ID No 1 to No 66 or 133-145 and at least one peptide of a second group, having SEQ ID No 159, or homologous sequences of polypeptides of the first group and/or the second group with a minimum of 25% of identity with the whole sequences of said polypeptides.

Preferred compositions comprise combinations with the polypeptide of the second group having SEQ ID No 159.

Others preferred compositions comprise combinations wherein the polypeptides of the first group have a sequence selected in the group comprising SEQ ID No 14, 15, 17, 21, 22, 23, 28, 29, 30, 32, 36, 38, 39, 41-44, 46, 49, 50, 52 to 55, 58, 60, 63, 133-138.

Other preferred compositions comprise the polypeptide having SEQ ID No 159 and a polypeptide selected in the group comprising peptides having sequences SEQ ID No 2, 26, 28, 36, 34, 134, 141 and 145.

The above-mentioned polypeptides of the first group and the polynucleotides coding for said polypeptides are disclosed in WO 03/074553 in the name of Mutabilis SA.

The polypeptide of SEQ ID No 159 and the polynucleotides having SEQ ID No 160 coding for said polypeptides are disclosed in WO 0121636 in the name of New-York University.

The invention also relates to combinations wherein said homologous isolated antigenic polypeptides of the first group, have at least 25% identity to a polypeptide having a sequence SEQ ID No as above defined, more particularly having SEQ ID No 14, 15, 17, 21, 22, 23, 28, 29, 30, 32, 36, 38, 39, 41-44, 46, 49, 50, 52 to 55, 58, 60, 63, 133-138, or at least 25% identity to a fragment comprising at least 5, at least 10, at least 20, at least 30, at least 40, at least 50, at least 60 or more than 60 consecutive amino acids of a polypeptide having a sequence corresponding to said SEQ ID Nos, as determined using BLASTP or BLASTX with the default parameters.

The invention also relates to combinations comprising homologous isolated antigenic peptides of second group having at least 25% identity to a polypeptide having SEQ ID No 159.

The invention also relates to the use in combination of isolated polynucleotides coding for a polypeptide of the first group and of isolated polynucleotides coding for polypeptides a polypeptide of the second group such as above defined according to the universal genetic code and taking into account the degeneracy of this code. The term “polynucleotide” emcompasses any nucleotidic sequence such as DNA, including cDNA, RNA, including mRNA.

The polynucleotides coding for the polypeptides of the first group have preferably sequences corresponding to SEQ ID No 67 to SEQ ID No 132 or 146 to 158 and are in combination with polynucleotide having SEQ ID No 160.

More preferably, said polynucleotides have sequences corresponding to SEQ ID No 80, 81, 83, 87, 88, 89, 94, 95, 96, 98, 102, 104, 105, 107-110, 112, 115, 116, 118, 119, 126, 127, 130, 132, 135, 146-151 and are in combination with polynucleotides having SEQ ID No 160.

Other preferred combinations comprise polynucleotides having polynucleotides having SEQ ID No 68, 92, 89, 94, 100, 154, 147 and 146 in combination with the polynucleotide having SEQ ID No 160.

The polynucleotides coding for the polypeptides of the second group have preferably sequence SEQ ID No 160.

The invention also relates to combinations of homologs to said polynucleotides. Said homologs may have at least 25% identity to a polynucleotide having said sequences, or at least 25% identity to a fragment comprising at least 15, at least 30, at least 60, at least 90, at least 120, at least 150, at least 180 or more than 180 consecutive nucleotide of a polynucleotide having one of said SEQ ID Nos, as determined using BLASTN with the default parameters, and are encompassed by the invention inasmuch as they are capable of coding for a polypeptide having the antigenic properties of those according to the invention.

The present application is also aimed towards any expression vector comprising at least one isolated polynucleotide coding for a polypeptide of said first group and at least one polynucleotide coding for a polypeptide of said second group according to the universal genetic code and taking into account the degeneracy of this code. The term “polynucleotide” encompasses any nucleotidic sequence such as DNA, including cDNA, RNA, including mRNA.

Preferred vectors comprise polynucleotides coding for the polypeptides of the first group having preferably sequences corresponding to SEQ ID No 77 to SEQ ID No 132 or 146 to 158.

More preferred vectors comprise, polynucleotides having sequences corresponding to SEQ ID No 80, 81, 83, 87, 88, 89, 94, 95, 96, 98, 102, 104, 105, 107-110, 112, 115, 116, 118, 119, 126, 127, 130, 132, 135, 146-151.

More preferred vectors further comprise polynucleotides coding for the polypeptides of the second group having sequence SEQ ID No 160.

Other preferred expression vectors comprise polynucleotides having SEQ ID No 68, 92, 89, 94, 100, 154, 147 and 146 in combination with the polynucleotide having SEQ ID No 160.

Said vectors may also comprise homologs to said polynucleotides. Said homologs may have at least 25% identity to a polynucleotide having said sequences, or at least 25% identity to a fragment comprising at least 15, at least 30, at least 60, at least 90, at least 120, at least 150, at least 180 or more than 180 consecutive nucleotide of a polynucleotide having one of said SEQ ID Nos, as determined using BLASTN with the default parameters, and are encompassed by the invention inasmuch as they are capable of coding for a polypeptide having the antigenic properties of those according to the invention.

The invention also relates to any cell transformed by genetic engineering, characterized in that it comprises, by transfection, at least one of polynucleotide coding for a polypeptide of said first group and at least one a polynucleotide coding for a polypeptide of said second group and/or at least one vector according to the invention, and/or in that said transformation induces the production by this cell of said polypeptides.

The combinations of said antigenic polypeptides are capable of inducing an antibody response for prevention of infections due to ExPEC strains regardless of the pathogenesis and of the infection site (UTI, pyelonephritis, sepsis, bacteremia, neonatal meningitis).

The invention thus relates to vaccine compositions specific to E. coli extra-intestinal infections, comprising an effective amount of at least one antigenic polypeptide or fragment thereof of said first group and at least one antigenic polypeptide or fragment thereof of the second group, with a carrier, particularly at least one polypeptide of SEQ ID No 1 to SEQ ID No 66 and 133-145 and the homologous polypeptides, and at least one polypeptide of SEQ ID No 159.

Such vaccine compositions are particularly useful for preventing urinary system infections, pyelonephritis, sepsis, bacteremia, neonatal meningitis.

The vaccine compositions of the invention are indicated for:
Immunodepressed patients, ideally before the start of the immunosuppressive therapy: patients suffering from cancer, diabetes, leukemia, transplant patients, patients receiving long-term steroids therapy.
Patients before surgery where there is a high risk of E. coli infections (abdominal surgery).
In all these cases, the E. coli vaccine of the invention could be administered in association with a Staphylococcus aureus vaccine or a group B Streptococcus vaccine,
Patients with recurrent UTI, especially after one episode of pyelonephritis,
The prevention of neonatal infections will require vaccination of the mother, implying vaccination long before pregnancy to avoid potential problem. Ideally such a vaccine should be associated with a Group B Streptococcus polysaccharide vaccine in order to also prevent late onset neonatal infections. It should be pointed out that the induction of a level of antibodies against B2/D E. coli in pregnant women would also prevent UTI, which are always a risk in the context of a pregnancy.

The formulation and the dose of said vaccine compositions can be developed and adjusted by those skilled in the art as a function of the indication targeted, of the method of administration desired, and of the patient under consideration (age, weight).

These compositions comprise one or more physiologically inert vehicles, and in particular any excipient suitable for the

For example the vaccine could be a suspension of the purified polypeptide in sterile water with aluminum based mineral salt as adjuvant and be administered subcutaneously with a first and boosting injection.

The combinations of antibodies respectively raised against at least one polypeptide of said first group and at least one polypeptide of said second group are also part of the invention.

They are capable of binding to said polypeptides in physiological-type conditions (in vivo or mimicking in vivo) when administered to a human or animal organism, and ELISA-type conditions when said binding product is intended to be used in assays and methods in vitro. Such combinations of antibodies advantageously inhibit the extra-intestinal growth of ExPEX strains in human or animal.

The invention thus relates to pharmaceutical compositions comprising an effective amount of a combination of antibodies such as above defined.

Such pharmaceutical compositions are particularly useful for immunotherapy applications for treatment and prevention of severe infections in at risk populations such as neonates or patients undergoing surgical procedures, or having urinary tract infections to prevent septicemia. For these applications specific human monoclonal antibody (Mab) will be derived from said peptides or polypeptides.

Such pharmaceutical compositions comprising an effective amount of a combination of antibodies such as above defined are also useful for treating neonatal infections, in association with antibodies against Staphylococcus aureus and/or antibodies against group B Streptococcus.

The methods for manufacturing such antibodies using the polypeptides of the combinations according to the invention are available to those skilled in the art. They are conventional methods which comprise, in particular, the immunization of animals such as rabbits and the harvesting of the serum produced, followed optionally by the purification of the serum obtained. A technique suitable for the production of monoclonal antibodies is that of Köhler and Milstein (Nature 1975, 256:495-497).

Said antibodies do not recognize the cells of the human or animal to which it is intended.

The antibodies or fragments thereof are advantageously humanized when intended for a human administration.

Alternatively, humanized Mab could be derived from murine or rat Mab specific of the antigen. These fully humanized Mab are constructed using conventional molecular techniques to graft complementarity-determining regions from the parent murine or rat antibacterial antibody into human IgG1 kappa heavy and light-chain frameworks.

The present invention is also aimed towards the use of said combinations of at least one polypeptide of the first group, particularly having SEQ ID No 14, 15, 17, 21, 22, 23, 28, 29, 30, 32, 36, 38, 39, 41-44, 46, 49, 50, 52 to 55, 58, 60, 63, 133-138, and one polypeptide of the second group, particularly having SEQ ID No 159, said antibodies raised against said polypeptides, or polynucleotides coding for said polypeptides for the diagnosis of the presence or absence of undesirable extra-intestinal E. coli, and/or for the diagnosis of an extra-intestinal E. coli infection.

The invention particularly relates to the use of said combinations of at least one polypeptide having SEQ ID No 14, 15, 17, 21, 22, 23, 28, 29, 30, 32, 36, 38, 39; 41-44, 46, 49, 50, 52 to 55, 58, 60, 63 or 133-138, and polypeptide having SEQ ID No 159, and use of antibodies raised against said polypeptides, or the use of polynucleotides coding for said polypeptides for the diagnosis of the presence or absence of undesirable extra-intestinal E. coli, and/or for the diagnosis of an extra-intestinal E. coli infection.

The invention also relates to the use of combinations of polypeptides comprising polypeptide having sequence SEQ ID No 159 and at least one polypeptide selected in the group comprising peptides having sequence SEQ ID No 2, 26, 28, 36, 34, 134, 141 and 145.

The detection of the presence or absence of such compounds can in particular be carried out by nucleotide hybridization, by PCR amplification or by detection of their polypeptide products. Detection of the presence of such compounds makes it possible to conclude that a B2/D E. coli strain is present.

The invention also relates to pharmaceutical compositions for alleviating and/or preventing and/or treating an undesirable growth of E. coli comprising an effective amount of at least one polypeptide of said each group particularly having SEQ ID No 1-66 to 133-145, for the first group, and SEQ ID No 159 for the second group, in combination with a pharmaceutically acceptable carrier.

Preferred pharmaceutical compositions comprise at least one polypeptide having SEQ ID No 14, 15, 17, 21, 22, 23, 28, 29, 30, 32, 36, 38, 39, 41-44, 46, 49, 50, 52 to 55, 58, 60, 63, 133-138, and at least one polypeptide having SEQ ID No 159.

Other preferred pharmaceutical compositions comprise at least one polypeptide having SEQ ID No 2, 26, 28, 36, 34, 134, 141 and 145 and polypeptide having SEQ ID No 159.

The present application is also aimed towards any use of said combination of polypeptides such as above defined for the manufacture of a composition, in particular of a pharmaceutical composition, intended to alleviate and/or to prevent and/or to treat an undesirable growth of E. coli, such as an E. coli infection, (for example systemic and non-diarrhoeal infections), the presence of extra-intestinal E. coli or a sanitary contamination.

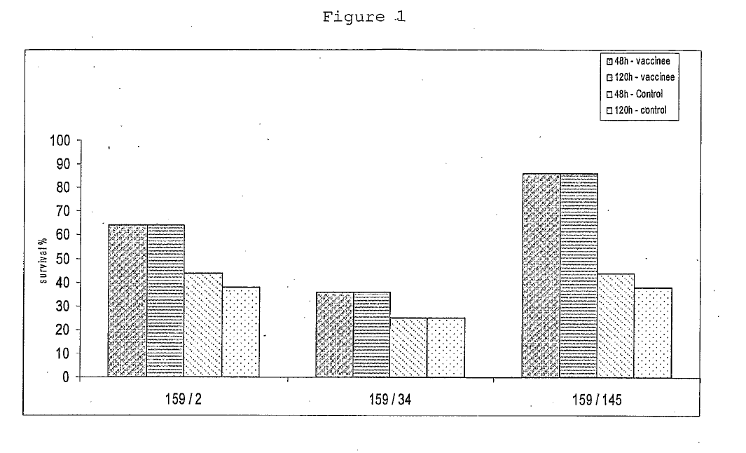

The present invention is illustrated by the examples, which follow and which are given in a non limiting capacity. In said examples, it is referred to FIG. 1 which represents histograms of the results showing an increase of survival in the animals vaccinated with polypeptides combinations.

Examples of vaccination to demonstrate immunogenicity of polypeptides:

### EXAMPLE 1

### Preparation of Antigenic Peptidic Combinations

A polypeptide having SEQ ID No 28 is purified from an E. coli strain or from an host cell containing a recombinant expression plasmide.

Polypeptides having SEQ ID No 28 and SEQ ID No 159, respectively, are purified and conjugated with a toxin.

A physiologically inert carrier is added to the preparation, which is sterilized and can be injected parenterally, subcutaneously or intramuscularly.

Said composition can also be sprayed onto mucosa with the aid of a spray.

Said combination of polypeptides may be added to a childhood vaccine.

Protecting Effect of Said Combination in Mice Infected by E. coli

A total of 100 μg of said purified combination of polypeptides was administered to Balb C mice according to usual procedure of immunization.

A decrease of mortality in immunized animals was observed compared to non-immunized animals.

### EXAMPLE 2

Example of antigens combination to induce an immune response that protects mice after experimental challenge with a pathogenic strain of E. coli (ExPEc):

### Experimental Protocol:

Balb/c mice, female, 6 weeks old were immunized on day 1 by subcutaneous injection of a solution containing a combination of two purified polypeptides (20 micrograms of each) and Complete Freund's adjuvant (CFA) in PBS, and control mice were injected with CFA in PBS buffer.
3 weeks later a boost injection of the same combination of polypeptides in solution (10 micrograms of each) with the incomplete Freund's adjuvant was performed.
Before challenge on day 42, sera was collected on day 41 to analyze the antibody response in the vaccinated animals:
WB analysis of sera from immunized mice were performed to detect the antibody response to the recombinant protein used for immunization as described above.
An ELISA assay was used to measure polypeptides specific antibody titres obtained in vaccinated animals:

Experimental challenge to measure protection induced by, antigens combination:

On day 42, vaccinated and control mice were challenged with an E. coli ExPEC virulent strain belonging to B2 group at a dose equal to the LD 50 (5.10⁵ cfu/mice) by intraperitoneal injection. The end point of the assay was the survival to the lethal challenge. Mortality observed in each group of animals was recorded at 48 h and 120 h. The results are shown in Table 1 are expressed as a percentage of survival in the vaccine group versus the control mice group.

TABLE 1
Protection obtained in mice challenged after immunization with
combination of polypeptides encoded by the corresponding ORFs.

| Combination of | % Survival at 48 h | | % Survival at 120 h | |
|---|---|---|---|---|
| two polypeptides | (nb of mice | | (nb of mice | |
| SEQ ID polypeptide 1/ | alive/nb total) | | alive/nb total) | |
| SEQ ID polypeptide 2 | Vaccine | Control | Vaccine | Control |
| 159/2 | 64 (9/14) | 44 (7/16) | 64 (9/14) | 38 (6/16) |
| 159/34 | 36 (5/14) | 25 (4/16) | 36 (5/14) | 25 (4/16) |
| 159/145 | 86 (12/14) | 44 (7/16) | 86 (12/14) | 38 (6/16) |

FIG. 1 represents histograms of the results and shows an increase of survival in the animals vaccinated with polypeptides combinations according to the invention.

What is claimed is:

1. Compositions of polypeptides specific to pathogenic strains comprising at least one polypeptide of a first group, having a sequence selected in the group comprising the sequences of SEQ ID No 1 to No 66 or 133-145 and at least one peptide of a second group, having SEQ ID No 159, or homologous sequences of polypeptides of the first group and/or the second group with a minimum of 25% of identity with the whole sequences of said polypeptides.

2. The compositions according to claim 1, wherein the polypeptides of the second group have SEQ ID No 159.

3. The compositions of claim 1, wherein the polypeptides of the first group have SEQ ID No 14, 15, 17, 21, 22, 23, 28, 29, 30, 32, 36, 38, 39, 41-44, 46, 49, 50, 52 to 55, 58, 60, 63 or 133-138.

4. The compositions according to claim 1, wherein the combination of two polypeptides comprises polypeptide having sequence SEQ ID No 159 and at least one polypeptide selected in the group comprising peptides having sequence SEQ ID No 2, 26, 28, 36, 34, 134, 141 and 145.

5. The compositions according to anyone of claims 1, wherein said homologues isolated antigenic polypeptides of the first group have at least 25% identity to a polypeptide having a sequence such as above defined in claim 1, more particularly having SEQ ID No 14, 15, 17, 21, 22, 23, 28, 29, 30, 32, 36, 38, 39, 41-44, 46, 49, 50, 52 to 55, 58, 60, 63, 133-138, or at least 25% identity to a fragment comprising at least
5, at least 10, at least 20, at least 30, at least 40, at least 50, at least 60 or more than 60 consecutive amino acids of a polypeptide having a sequence corresponding to said SEQ ID Nos, as determined using BLASTP or BLASTX with the default parameters.

6. The compositions according to claim 1, wherein said homologous isolated antigenic polypeptides of the second group have at least 25% identity to a polypeptide having SEQ ID No 159.

7. Use in combination of isolated polynucleotides coding for a polypeptide of the first group and of isolated polynucleotides coding for a polypeptide of the second group as defined in claim 1 according to the universal genetic code and taking into account the degeneracy of this code.

8. The use of claim 7, comprising polynucleotides coding for the polypeptides of said first group and having sequences corresponding to SEQ ID No 67 to SEQ ID No 132 or 146 to 158 in combination with polynucleotide having SEQ ID No 160.

9. The use of claim 8, comprising, the nucleotides having sequences corresponding to SEQ ID No 80, 81, 83, 87, 88, 89, 94, 95, 96, 98, 102, 104, 105, 107-110, 112, 115, 116, 118, 119, 126, 127, 130, 132, 135, 146-151 in combination with the polynucleotide having SEQ ID No 160.

10. The use of claim 7, comprising polynucleotides having SEQ ID No 68, 92, 89, 94, 100, 154, 147 and 146 in combination with the polynucleotide having SEQ ID No 160.

11. The use of claim 7, comprising homologs of said polynucleotides having at least 25% identity to a fragment comprising at least 15, at least 30, at least 60, at least 90, at least 120, at least 150, at least 180 or more than 180 consecutive nucleotide of a polynucleotide having one of said SEQ ID Nos, as determined using BLASTN with the default parameters, inasmuch as they are capable of coding for a polypeptide having antigenic properties of those according to the invention.

12. An expression vector comprising at least one isolated polynucleotide coding for a polypeptide of said first group and at least one polypeptide of said second group according to the universal genetic code and taking into account the degeneracy of this code, said groups being as defined in claim 1.

13. The expression vector according to claim 12, wherein the polynucleotides coding for the polypeptides of the first group have sequences corresponding to SEQ ID No 67 to SEQ ID No 132 or 146 to 158.

14. The expression vector according to claim 12, wherein said polynucleotides have sequences corresponding to SEQ ID No 80, 81, 83, 87, 88, 89, 94, 95, 96, 98, 102, 104, 105, 107-110, 112, 115, 116, 118, 119, 126, 127, 130, 132, 135, 146-151.

15. The expression vector according to claim 12, wherein the polynucleotide coding for the polypeptide of the second group has SEQ ID No 160.

16. The expression vector of anyone of claim 13, comprising polynucleotides having SEQ ID No 68, 92, 89, 94, 100, 154, 147 and 146 in combination with the polynucleotide having SEQ ID No 160.

17. An expression vector according to claim 12, comprising an homolog to said polynucleotides, said homologs having at least 25% identity to a fragment comprising at least 15, at least 30, at least 60, at least 90, at least 120, at least 150, at least 180 or more than 180 consecutive nucleotide of a polynucleotide having one of said SEQ ID Nos, as determined using BLASTN with the default parameters, and are encompassed by the invention inasmuch as they are capable of coding for a polypeptide having the antigenic properties of those according to the invention.

18. An expression vector according to claim 12, comprising polynucleotides having SEQ ID No 68, 92, 89, 94, 100, 154, 147, 146 and the polynucleotide having SEQ ID No 160.

19. A host cell comprising an expression vector according to claim 12.

20. Vaccine compositions specific to E. coli extra-intestinal infections, comprising an effective amount of at least one antigenic polypeptide or fragment thereof of said first group and at least one antigenic polypeptide or fragment thereof of the second group, with a carrier, particularly at least one polypeptide of SEQ ID No 1 to SEQ ID No 66 and 133-145 and homologous polypeptides, and at least one polypeptide of SEQ ID No 159 and homologous peptides.

21. The vaccine compositions of claim 14, for preventing urinary system infections, pyelonephritis, sepsis, bacteremia, neonatal meningitidis.

22. The vaccine composition of claim 20, adapted to specific indication in combination with components directed against other bacteria, such as S. Aureus or group B Streptococcus, or other bacteria implicated in systemic infections.

23. Compositions of antibodies specific to polypeptidic antigens of pathogenic strains particularly to extra-intestinal E. Coli strains, comprising combinations of antibodies directed against at least one polypeptide of said first group and antibodies directed against at least one polypeptide of the second group such as defined in claim 1.

24. Compositions according to claim 23, wherein said antibodies are monoclonal antibodies.

25. Pharmaceutical compositions comprising a combination of antibodies according to claim 23.

26. Pharmaceutical compositions according to claim 25 comprising an effective amount of a combination of antibodies for treating neonatal infections, in association with antibodies against Staphylococcus aureus and/or antibodies against group B Streptococcus.

27. The use of a pharmaceutical composition according to claim 25 for treatment or prevention of severe infection due to Expec in neonates and patients at risk for such infections.

28. Pharmaceutical compositions for alleviating and/or preventing and/or treating an undesirable growth of E. Coli comprising an effective amount of at least a composition according to claim 1, in combination with a pharmaceutically acceptable carrier.
